SEQUENCE LISTING (28 sequences)

SEQ ID NO: 1 - DNA, 1355 bp, Homo sapiens
gctagctggcatggcggcctgcattgcagcggggcactgggctgcaatgggcctaggccggagtttccaagccgccaggactctgctccccccgccggcctctatcgcctgcagggtccacgcggggcctgtccggcagcagagcactgggccttccgagcccggtgcgttccaaccgccgccgaaaccggtcatcgtggacaagcaccgccccgtggaaccggaacgcaggttcttgagtcctgaattcattcctcgaaggggaagaacagatcctctgaaatttcaaatagaaagaaaagatatgttagaaaggagaaaagtactccacattccagagttctatgttggaagtattcttcgtgttactacagctgacccatatgccagtggaaaaatcagccagtttctggggatttgcattcagagatcaggaagaggacttggagctactttcatccttaggaatgttatcgaaggacaaggtgtcgagatttgctttgaactttataatcctcgggtccaggagattcaggtggtcaaattagagaaacggctggatgatagcttgctatacttacgagatgcccttcctgaatatagcacttttgatgtgaatatgaagccagtagtacaagagcctaaccaaaaagttcctgttaatgagctgaaagtaaaaatgaagcctaagccctggtctaaacgctgggaacgtccaaattttaatattaaaggaatcagatttgatctttgtttaactgaacagcaaatgaaagaagctcagaagtggaatcagccatggcttgaatttgatatgatgagggaatatgatacttcaaaaattgaagctgcaatatggaaggaaattgaagcgtcgaaaaggtcttgattctgagaatgaatttggttagttgcagaagatacattggctctaagaggatatattttgagaccaatttaatttcatttataagaacatagtaattaagtgaactaagcattcattgttttattaatactttttttctaaaataaaacttgtacaccagtttattactctaaaaagagaattacacatgccaaatggaccaatgtccatttgcttattggaggcaaagctacaatagaagtcagagcatcaccagaatggtctttaatgagcatggaacctgagcaaagggaataggtgggatgaattttttttttaattgtgaaacaattcataagcacaatatgatttacagaataataaacattcatgtacccactatcaggttaagaaatagaacatttattaatatgtaggaatgttaagaaataaaacatttaataagaaaaaaaaaaaaaaaaaaaaa

SEQ ID NO: 2 - DNA, 1433 bp, Homo sapiens
cagaggccggcttggtcactatggaggagataggcatcttggtggagaaggctcaggatgagatcccagcactgtccgtgtcccggccccagaccggcctgtccttcctgggccctgagcctgaggacctggaggacctgtacagccgctacaagaagctgcagcaagagctggagttcctggaggtgcaggaggaatacatcaaagatgagcaaaagaacctgaaaaaggaatttctccatgcccaggaggaggtgaagcgaatccaaagcatcccgctggtcatcggacaatttctggaggctgtggatcagaatacagccatcgtgggctctaccacaggctccaactattatgtgcgcatcctgagcaccatcgatcgggagctgctcaagcccaacgcctcagtggccctccacaagcacagcaatgcactggtggacgtgctgccccccgaagccgacagcagcatcatgatgctcacctcagaccagaagccagatgtgatgtacgcggacatcggaggcatggacatccagaagcaggaggtgcgggaggccgtggagctcccgctcacgcatttcgagctctacaagcagatcggcatcgatcccccccgaggcgtcctcatgtatggcccacctggctgtgggaagaccatgttggcaaaggcggtggcacatcacacaacagctgcattcatccgggtcgtgggctcggagtttgtacagaagtatctgggtgagggcccccgcatggtccgggatgtgttccgcctggccaaggagaatgcacctgccatcatcttcatagacgagattgatgccatcgccaccaagagattcgatgctcagacaggggccgacagggaggttcagaggatcctgctggagctgctgaatcagatggatggatttgatcagaatgtcaatgtcaaggtaatcatggccacaaacagagcagacaccctggatccggccctgctacggccaggacggctggaccgtaaaattgaatttccacttcctgaccgccgccagaagagattgattttctccactatcactagcaagatgaacctctctgaggaggttgacttggaagactatgtggcccggccagataagatttcaggagctgatattaactccatctgtcaggagagtggaatgttggctgtccgtgaaaaccgctacattgtcctggccaaggacttcgagaaagcatacaagactgtcatcaagaaggacgagcaggagcatgagttttacaagtgacccttcccttccctccaccacaccactcaggggctggggcttctctcgcacccccagcacctctgtcccaaaacctcattcccttttttctttacccaggattggtttcttcaataaatagataagatcgaatccaaaaaaaaaaaaaaaaaaaaa

SEQ ID NO: 3 - DNA, 2935 bp, Homo sapiens
ccgcaactcgctcggccgccgccatcttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattcaaattgtcctgcagaggttcattcccctgaccctttccccacattggtaagagtagctgggttttctaagccactctctggaatctctttgtgttagggtctcgatttgaggacattcatttcttcagcagcccattagcaactgagagcccagggatgtcctacaggatagtttcatagtgacaggtggcacttggctaatagaatatggctgatattgtcattaatcattttgtaccttgacatgggttgtctaataaaactcggacccttcttgtgaaaaaaaaaaaaaaaaaa

SEQ ID NO: 4 - DNA, 4016 bp, Homo sapiens
gagcgccagagagagaagatcggggggctgaaatccatcttcatcctaccgctccgcccgtgttggtggaatgagcgttgcatgtgtcttgaagagaaaagcagtgctttggcaggactctttcagcccccacctgaaacatcaccctcaagaaccagctaatcccaacatgcctgttgttttgacatctggaacagggtcgcaagcgcagccacaaccagctgcaaatcaggctcttgcagctgggactcactccagccctgtcccaggatctataggagttgcaggccgttcccaggacgacgctatggtggactacttctttcagaggcagcatggtgagcagcttgggggaggaggaagtggaggaggcggctataataatagcaaacatcgatggcctactggggataacattcatgcagaacatcaggtgcgttccatggatgaactgaatcatgattttcaagcacttgctctggagggaagagcgatgggagagcagctcttgccaggtaaaaagttttgggaaacagatgaatccagcaaagatggaccaaaaggaatattcctgggtgatcaatggcgagacagtgcctggggaacatcagatcattcagtttcccagccaatcatggtgcagagaagacctggtcagagtttccatgtgaacagtgaggtcaattctgtactgtccccacgatcggagagtgggggactaggcgttagcatggtggagtatgtgttgagctcatccccgggcgattcctgtctaagaaaaggaggatttggcccaagggatgcagacagtgatgaaaacgacaaaggtgaaaagaagaacaagggtacgtttgatggagataagctaggagatttgaaggaggagggtgatgtgatggacaagaccaatggtttaccagtgcagaatgggattgatgcagacgtcaaagattttagccgtacccctggtaattgccagaactctgctaatgaagtggatcttctgggtccaaaccagaatggttctgagggcttagcccagctgaccagcaccaatggtgccaagcctgtggaggatttctccaacatggagtcccagagtgtccccttggaccccatggaacatgtgggcatggagcctcttcagtttgattattcaggcacgcaggtacctgtggactcagcagcagcaactgtgggactttttgactacaattctcaacaacagctgttccaaagacctaatgcgcttgctgtccagcagttgacagctgctcagcagcagcagtatgcactggcagctgctcatcagccgcacatcggtttagctcccgctgcgtttgtccccaatccatacatcatcagcgctgctcccccagggacggacccctacacagctggattggctgcagcagcgacactaggcccagctgtggtccctcaccagtattatggagttactccctggggagtctaccctgccagtcttttccagcagcaagctgccgctgccgctgcagcaactaattcagctaatcaacagaccaccccacaggctcagcaaggacagcagcaggttctccgtggaggagccagccaacgtcctttgaccccaaaccagaaccagcagggacagcaaacggatccccttgtggcagctgcagcagtgaattctgcccttgcatttggacaaggtctggcagcaggcatgccaggttatccggtgttggctcctgctgcttactatgaccaaactggtgcccttgtagtgaatgcaggcgcgagaaatggtcttggagctcctgttcgacttgtagctcctgccccagtcatcattagttcctcagctgcacaagcagctgttgcagcagccgcagcttcagcaaatggagcagctggtggtcttgctggaacaacaaatggaccatttcgccctttaggaacacagcagcctcagccccagccccagcagcagcccaataacaacctggcatccagttctttctacggcaacaactctctgaacagcaattcacagagcagctccctcttctcccagggctctgcccagcctgccaacacatccttgggattcggaagtagcagttctctcggcgccaccctgggatccgcccttggagggtttggaacagcagttgcaaactccaacactggcagtggctcccgccgtgactccctgactggcagcagtgacctttataagaggacatcgagcagcttgacccccattggacacagtttttataacggccttagcttttcctcctctcctggacccgtgggcatgcctctccctagtcagggaccaggacattcacagacaccacctccttccctctcttcacatggatcctcttcaagcttaaacctgggaggactcacgaacggcagtggaagatacatctctgctgctccaggcgctgaagccaagtaccgcagtgcaagcagcgcctccagcctcttcagcccgagcagcactcttttctcttcctctcgtttgcgatatggaatgtctgatgtcatgccttctggcaggagcaggcttttggaagattttcgaaacaaccggtaccccaatttacaactgcgggagattgctggacatataatggaattttcccaagaccagcatgggtccagattcattcagctgaaactggagcgtgccacaccagctgagcgccagcttgtcttcaatgaaatcctccaggctgcctaccaactcatggtggatgtgtttggtaattacgtcattcagaagttctttgaatttggcagtcttgaacagaagctggctttggcagaacggattcgaggccacgtcctgtcattggcactacagatgtatggctgccgtgttatccagaaagctcttgagtttattccttcagaccagcagaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatccttatggctgccgagtgattcagagaatcctggagcactgtctccctgaccagacactccctattttagaggagcttcaccagcacacagagcagcttgtacaggatcaatatggaaattatgtaatccaacatgtactggagcacggtcgtcctgaggataaaagcaaaattgtagcagaaatccgaggcaatgtacttgtattgagtcagcacaaatttgcaagcaatgttgtggagaagtgtgttactcacgcctcacgtacggagcgcgctgtgctcatcgatgaggtgtgcaccatgaacgacggtccccacagtgccttatacaccatgatgaaggaccagtatgccaactacgtggtccagaagatgattgacgtggcggagccaggccagcggaagatcgtcatgcataagatccggccccacatcgcaactcttcgtaagtacacctatggcaagcacattctggccaagctggagaagtactacatgaagaacggtgttgacttagggcccatctgtggcccccctaatggtatcatctgaggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaaaaaaaaaa

SEQ ID NO: 5 - DNA, 7399 bp, Homo sapiens
acctgacaacctctcatccctgctctgcagatccggtcccatccccactgcccaccccacccccccagcactccacccagttcaacgttccacgaacccccagaaccagccctcatcaacaggcagcaagaagggccccccgcccatcgccccacaacgccagccgggtgaactgtagcgttggcaggtcctgaggcagctggcaagacgcctgcagctgaaagatacaaggccagggacaggacagtcccatccccaggaggcagggagtatacaggctggggaagtttgcccttgcgtggggtggtgatggaggaggctcagcaagtcttctggactgtgaacctgtgtctgccactgtgtgctgggtggtggtcatctttcccaccaggctgtggcctctgcaaccttcaagggaggagcaggtcccattggctgagcacagccttgtaccgtgaactggaacaagcagcctccttcctggccacaggttccatgtccttatatggactcatctttgcctattgcgacacacactcagtgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggccttgtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctattctcgcagctcaccatggatgatgatatcgccgcgctcgtcgtcgacaacggctccggcatgtgcaaggccggcttcgcgggcgacgatgccccccgggccgtcttcccctccatcgtggggcgccccaggcaccaggtaggggagctggctgggtggggcagccccgggagcgggcgggaggcaagggcgctttctctgcacaggagcctcccggtttccggggtgggggctgcgcccgtgctcagggcttcttgtcctttccttcccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccaggtgagtggcccgctacctcttctggtggccgcctccctccttcctggcctcccggagctgcgccctttctcactggttctctcttctgccgttttccgtaggactctcttctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggcgcacagtaggtctgaacagactccccatcccaagaccccagcacacttagccgtgttctttgcactttctgcatgtcccccgtctggcctggctgtccccagtggcttccccagtgtgacatggtgtatctctgccttacagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggtgagtggagactgtctcccggctctgcctgacatgagggttacccctcggggctgtgctgtggaagctaagtcctgccctcatttccctctcaggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaaggtgggtgtctttcctgcctgagctgacctgggcaggtcggctgtggggtcctgtggtgtgtggggagctgtcacatccagggtcctcactgcctgtccccttccctcctcagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaggccaagtgtgactttgtggtgtggctgggttgggggcagcagagggtgaaccctgcaggagggtgaaccctgcaaaagggtggggcagtgggggccaacttgtccttacccagagtgcaggtgtgtggagatccctcctgccttgacattgagcagccttagagggtgggggaggctcaggggtcaggtctctgttcctgcttattggggagttcctggcctggcccttctatgtctccccaggtaccccagtttttctgggttcacccagagtgcagatgcttgaggaggtgggaagggactatttgggggtgtctggctcaggtgccatgcctcactggggctggttggcacctgcatttcctgggagtggggctgtctcagggtagctgggcacggtgttcccttgagtgggggtgtagtgggtgttcctagctgccacgcctttgccttcacctatgggatcgtggctgtcagccttgagggtcagcctggcccaggctcccataggcttaggagaggccgcaattcctacctgttcatccagacagagggggacctggaatcaaagtcaagttggggtagggggtccatggggccatatctggcctgcagacagctctggttagctatgggctgaggtctggattctgccttgtgactggagactgggcgccatcccgtggcctctgagggcccagtttgttcgtgacccatgctgcagagggagatacccaggctcctggacctcagggcctgcctggcactgcaggccaggttctctttttatttctgagacggggtcttgtgttgcccaggctggagtgtaatggcacaaccatggctcactgcagcctcaactttgggctcaagcaattctcctgcctcagcctcccaagtagctgaaactacacaggcatgccccagcacgcccagctagttgcaggtcagattctcattgttctacagggccctgagttaaatactctcgcaagaagaaaccaagcctggcctgccttccctctgctagaagctgtgattgacaccagcagagaatgaaggaagaaaagggggtgaggatcccttgcagggatgccgtttaccccaaacaccacctggtgcatagcagttgttctggaaatgatccggtccccccaggaggcctggactctcaactgtgcacagtggcctactggcagagaggaaggtggtacccttcctcctgtgttatcttggaggtcccctgaagggctctctggggtggttctagctcttttggtctgatttctgcagtggtgagccctgggccaccttgggccacttaatcattcaacccccattcattgagtgaccactctacggtgggggaggctactgagatgacctggacaaggaccctgccctggaggagctgtcagtctggaagtgaggatttaattcatttgcccattcattcattcattcattcattcattgaggacttaattcatttgctcattcattcattcatttgctcattcatccattcattcactcattcatccattcattcatccatccatccatccatccatccaatcactatttgtgccagatagtgcgggtatagaggagaaccaaccagatgtgttccgtgtcctcaccaggcacccagtttaatggggaagacatcacaagcacgaaggcaaacaaatgagtgtacagtgacatgctgtgatcataggaagagagagcagagactttgatgaacacgagcatcgggtgatgttcattatgtcgattgtggtggtggtcttgcaggtgtagacatacttcaaaccttatctaattgggcctgctgtggtgacgcatgcttgtaatcccagcattttgggaagctgaggtgggaggatcacttgagcccaggagctcaaggctgcagtgagctatgattgcaccactgcactccagcctgggtgacagaacaagaacctgactcttaaaaattaaaataggccaggcaggcgtggtg

SEQ ID NO: 6 - DNA, 1307 bp, Homo sapiens
gccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaaaaaaaaaaaaaaaaacaaaaaaaaaaaaaaaaaaaaaaaaaaaaaaaaaaaaaaaaaaaaaaaaaaaaaaaaa

SEQ ID NO: 7 - DNA, 470 bp, Homo sapiens
atgtgtcactctcgcagctgccacccgaccatgaccatcctgcaggccccgaccccggccccctccaccatcccgggaccccggcggggctccggtcctgagatcttcaccttcgacccttcccggagcccgcagcggcccctgccgggcgccccagcgcctctcgcgggcaccgaaagcgcagccgcagggttctctaccctcgagtggtccggcgccagctgccagtcgaggaaccgaacccagccaaaaggcttctctttctgctgctcaccatcgtcttctgccagatcctgatggctgaagagggtgtgccggcgcccctgcctccagaggacgcccctaacgccgcatccctggcgcccacccctgtgtcccccgtcctcgagccctttaatctgacttcggagccctcggactacgctctggacctcagcactttcctccagcaacacccggccgccttctag

SEQ ID NO: 8 - DNA, 1172 bp, Homo sapiens
cccgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaaaaaaaaaaaaaaaaaaaaaaaaaaaaa

SEQ ID NO: 9 - protein, 292 aa, Homo sapiens [sequence omitted]

SEQ ID NO: 10 - protein, 418 aa, Homo sapiens
MEEIGILVEKAQDEIPALSVSRPQTGLSFLGPEPEDLEDLYSRYKKLQQELEFLEVQEEYIKDEQKNLKKEFLHAQEEVKRIQSIPLVIGQFLEAVDQNTAIVGSTTGSNYYVRILSTIDRELLKPNASVALHKHSNALVDVLPPEADSSIMMLTSDQKPDVMYADIGGMDIQKQEVREAVELPLTHFELYKQIGIDPPRGVLMYGPPGCGKTMLAKAVAHHTTAAFIRVVGSEFVQKYLGEGPRMVRDVFRLAKENAPAIIFIDEIDAIATKRFDAQTGADREVQRILLELLNQMDGFDQNVNVKVIMATNRADTLDPALLRPGRLDRKIEFPLPDRRQKRLIFSTITSKMNLSEEVDLEDYVARPDKISGADINSICQESGMLAVRENRYIVLAKDFEKAYKTVIKKDEQEHEFYK

SEQ ID NO: 11 - DNA, 1363 bp, Homo sapiens
ggggatcccaggccacacagaggccggcttggtcactatggaggagataggcatcttggtggagaaggctcaggatgagatcccagcactgtccgtgtcccggccccagaccggcctgtccttcctgggccctgagcctgaggacctggaggacctgtacagccgctacaaggaggaggtgaagcgaatccaaagcatcccgctggtcatcggacaatttctggaggctgtggatcagaatacagccatcgtgggctctaccacaggctccaactattatgtgcgcatcctgagcaccatcgatcgggagctgctcaagcccaacgcctcagtggccctccacaagcacagcaatgcactggtggacgtgctgccccccgaagccgacagcagcatcatgatgctcacctcagaccagaagccagatgtgatgtacgcggacatcggaggcatggacatccagaagcaggaggtgcgggaggccgtggagctcccgctcacgcatttcgagctctacaagcagatcggcatcgatcccccccgaggcgtcctcatgtatggcccacctggctgtgggaagaccatgttggcaaaggcggtggcacatcacacaacagctgcattcatccgggtcgtgggctcggagtttgtacagaagtatctgggtgagggcccccgcatggtccgggatgtgttccgcctggccaaggagaatgcacctgccatcatcttcatagacgagattgatgccatcgccaccaagagattcgatgctcagacaggggccgacagggaggttcagaggatcctgctggagctgctgaatcagatggatggatttgatcagaatgtcaatgtcaaggtaatcatggccacaaacagagcagacaccctggatccggccctgctacggccaggacggctggaccgtaaaattgaatttccacttcctgaccgccgccagaagagattgattttctccactatcactagcaagatgaacctctctgaggaggttgacttggaagactatgtggcccggccagataagatttcaggagctgatattaactccatctgtcaggagagtggaatgttggctgtccgtgaaaaccgctacattgtcctggccaaggacttcgagaaagcatacaagactgtcatcaagaaggacgagcaggagcatgagttttacaagtgacccttcccttccctccaccacaccactcaggggctggggcttctctcgcacccccagcacctctgtcccaaaacctcattcccttttttctttacccaggattggtttcttcaataaatagataagatcgaattcaaaaaaaaaaaaaaaaaaaaaaaaaaa

SEQ ID NO: 12 - protein, 387 aa, Homo sapiens
MEEIGILVEKAQDEIPALSVSRPQTGLSFLGPEPEDLEDLYSRYKEEVKRIQSIPLVIGQFLEAVDQNTAIVGSTTGSNYYVRILSTIDRELLKPNASVALHKHSNALVDVLPPEADSSIMMLTSDQKPDVMYADIGGMDIQKQEVREAVELPLTHFELYKQIGIDPPRGVLMYGPPGCGKTMLAKAVAHHTTAAFIRVVGSEFVQKYLGEGPRMVRDVFRLAKENAPAIIFIDEIDAIATKRFDAQTGADREVQRILLELLNQMDGFDQNVNVKVIMATNRADTLDPALLRPGRLDRKIEFPLPDRRQKRLIFSTITSKMNLSEEVDLEDYVARPDKISGADINSICQESGMLAVRENRYIVLAKDFEKAYKTVIKKDEQEHEFYK

SEQ ID NO: 13 - protein, 793 aa, Homo sapiens
MPAGPVQAVPPPPPVPTEPKQPTEEEASSKEDSAPSKPVVGIIYPPPEVRNIVDKTASFVARNGPEFEARIRQNEINNPKFNFLNPNDPYHAYYRHKVSEFKEGKAQEPSAAIPKVMQQQQQTTQQQLPQKVQAQVIQETIVPKEPPPEFEFIADPPSISAFDLDVVKLTAQFVARNGRQFLTQLMQKEQRNYQFDFLRPQHSLFNYFTKLVEQYTKILIPPKGLFSKLKKEAENPREVLDQVCYRVEWAKFQERERKKEEEEKEKERVAYAQIDWHDFVVVETVDFQPNEQGNFPPPTTPEELGARILIQERYEKFGESEEVEMEVESDEEDDKQEKAEEPPSQLDQDTQVQDMDEGSDDEEEGQKVPPPPETPMPPPLPPTPDQVIVRKDYDPKASKPLPPAPAPDEYLVSPITGEKIPASKMQEHMRIGLLDPRWLEQRDRSIREKQSDDEVYAPGLDIESSLKQLAERRTDIFGVEETAIGKKIGEEEIQKPEEKVTWDGHSGSMARTQQAAQANITLQEQIEAIHKAKGLVPEDDTKEKIGPSKPNEIPQQPPPPSSATNIPSSAPPITSVPRPPTMPPPVRTTVVSAVPVMPRPPMASVVRLPPGSVIAPMPPIIHAPRINVVPMPPSAPPIMAPRPPPMIVPTAFVPAPPVAPVPAPAPMPPVHPPPPMEDEPTSKKLKTEDSLMPEEEFLRRNKGPVSIKVQVPNMQDKTEWKLNGQVLVFTLPLTDQVSVIKVKIHEATGMPAGKQKLQYEGIFIKDSNSLAYYNMANGAVIHLALKERGGRKK

SEQ ID NO: 14 - DNA, 2910 bp, Homo sapiens
tgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattcaaattgtcctgcagaggttcattcccctgaccctttccccacattggtaagagtagctgggttttctaagccactctctggaatctctttgtgttagggtctcgatttgaggacattcatttcttcagcagcccattagcaactgagagcccagggatgtcctacaggatagtttcatagtgacaggtggcacttggctaatagaatatggctgatattgtcattaatcattttgtaccttgacatgggttgtctaataaaactcggacccttcttgtgaaaaaaaaaaaaaaaaaaaa

SEQ ID NO: 15 - protein, 793 aa, Homo sapiens; identical to SEQ ID NO: 13

SEQ ID NO: 16 - DNA, 2935 bp, Homo sapiens; identical to SEQ ID NO: 3

SEQ ID NO: 17 - protein, 793 aa, Homo sapiens; identical to SEQ ID NO: 13

SEQ ID NO: 18 - protein, 1186 aa, Homo sapiens
MSVACVLKRKAVLWQDSFSPHLKHHPQEPANPNMPVVLTSGTGSQAQPQPAANQALAAGTHSSPVPGSIGVAGRSQDDAMVDYFFQRQHGEQLGGGGSGGGGYNNSKHRWPTGDNIHAEHQVRSMDELNHDFQALALEGRAMGEQLLPGKKFWETDESSKDGPKGIFLGDQWRDSAWGTSDHSVSQPIMVQRRPGQSFHVNSEVNSVLSPRSESGGLGVSMVEYVLSSSPGDSCLRKGGFGPRDADSDENDKGEKKNKGTFDGDKLGDLKEEGDVMDKTNGLPVQNGIDADVKDFSRTPGNCQNSANEVDLLGPNQNGSEGLAQLTSTNGAKPVEDFSNMESQSVPLDPMEHVGMEPLQFDYSGTQVPVDSAAATVGLFDYNSQQQLFQRPNALAVQQLTAAQQQQYALAAAHQPHIGLAPAAFVPNPYIISAAPPGTDPYTAGLAAAATLGPAVVPHQYYGVTPWGVYPASLFQQQAAAAAAATNSANQQTTPQAQQGQQQVLRGGASQRPLTPNQNQQGQQTDPLVAAAAVNSALAFGQGLAAGMPGYPVLAPAAYYDQTGALVVNAGARNGLGAPVRLVAPAPVIISSSAAQAAVAAAAASANGAAGGLAGTTNGPFRPLGTQQPQPQPQQQPNNNLASSSFYGNNSLNSNSQSSSLFSQGSAQPANTSLGFGSSSSLGATLGSALGGFGTAVANSNTGSGSRRDSLTGSSDLYKRTSSSLTPIGHSFYNGLSFSSSPGPVGMPLPSQGPGHSQTPPPSLSSHGSSSSLNLGGLTNGSGRYISAAPGAEAKYRSASSASSLFSPSSTLFSSSRLRYGMSDVMPSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGPICGPPNGII

SEQ ID NO: 19 - DNA, 5390 bp, Homo sapiens
gggggtgaaaggtaagggggagcgagagcgccagagagagaagatcggggggctgaaatccatcttcatcctaccgctccgcccgtgttggtggaatgagcgttgcatgtgtcttgaagagaaaagcagtgctttggcaggactctttcagcccccacctgaaacatcaccctcaagaaccagctaatcccaacatgcctgttgttttgacatctggaacagggtcgcaagcgcagccacaaccagctgcaaatcaggctcttgcagctgggactcactccagccctgtcccaggatctataggagttgcaggccgttcccaggacgacgctatggtggactacttctttcagaggcagcatggtgagcagcttgggggaggaggaagtggaggaggcggctataataatagcaaacatcgatggcctactggggataacattcatgcagaacatcaggtgcgttccatggatgaactgaatcatgattttcaagcacttgctctggagggaagagcgatgggagagcagctcttgccaggtaaaaagttttgggaaacagatgaatccagcaaagatggaccaaaaggaatattcctgggtgatcaatggcgagacagtgcctggggaacatcagatcattcagtttcccagccaatcatggtgcagagaagacctggtcagagtttccatgtgaacagtgaggtcaattctgtactgtccccacgatcggagagtgggggactaggcgttagcatggtggagtatgtgttgagctcatccccgggcgattcctgtctaagaaaaggaggatttggcccaagggatgcagacagtgatgaaaacgacaaaggtgaaaagaagaacaagggtacgtttgatggagataagctaggagatttgaaggaggagggtgatgtgatggacaagaccaatggtttaccagtgcagaatgggattgatgcagacgtcaaagattttagccgtacccctggtaattgccagaactctgctaatgaagtggatcttctgggtccaaaccagaatggttctgagggcttagcccagctgaccagcaccaatggtgccaagcctgtggaggatttctccaacatggagtcccagagtgtccccttggaccccatggaacatgtgggcatggagcctcttcagtttgattattcaggcacgcaggtacctgtggactcagcagcagcaactgtgggactttttgactacaattctcaacaacagctgttccaaagacctaatgcgcttgctgtccagcagttgacagctgctcagcagcagcagtatgcactggcagctgctcatcagccgcacatcggtttagctcccgctgcgtttgtccccaatccatacatcatcagcgctgctcccccagggacggacccctacacagctggattggctgcagcagcgacactaggcccagctgtggtccctcaccagtattatggagttactccctggggagtctaccctgccagtcttttccagcagcaagctgccgctgccgctgcagcaactaattcagctaatcaacagaccaccccacaggctcagcaaggacagcagcaggttctccgtggaggagccagccaacgtcctttgaccccaaaccagaaccagcagggacagcaaacggatccccttgtggcagctgcagcagtgaattctgcccttgcatttggacaaggtctggcagcaggcatgccaggttatccggtgttggctcctgctgcttactatgaccaaactggtgcccttgtagtgaatgcaggcgcgagaaatggtcttggagctcctgttcgacttgtagctcctgccccagtcatcattagttcctcagctgcacaagcagctgttgcagcagccgcagcttcagcaaatggagcagctggtggtcttgctggaacaacaaatggaccatttcgccctttaggaacacagcagcctcagccccagccccagcagcagcccaataacaacctggcatccagttctttctacggcaacaactctctgaacagcaattcacagagcagctccctcttctcccagggctctgcccagcctgccaacacatccttgggattcggaagtagcagttctctcggcgccaccctgggatccgcccttggagggtttggaacagcagttgcaaactccaacactggcagtggctcccgccgtgactccctgactggcagcagtgacctttataagaggacatcgagcagcttgacccccattggacacagtttttataacggccttagcttttcctcctctcctggacccgtgggcatgcctctccctagtcagggaccaggacattcacagacaccacctccttccctctcttcacatggatcctcttcaagcttaaacctgggaggactcacgaatggcagtggaagatacatctctgctgctccaggcgctgaagccaagtaccgcagtgcaagcagcgcctccagcctcttcagcccgagcagcactcttttctcttcctctcgtttgcgatatggaatgtctgatgtcatgccttctggcaggagcaggcttttggaagattttcgaaacaaccggtaccccaatttacaactgcgggagattgctggacatataatggaattttcccaagaccagcatgggtccagattcattcagctgaaactggagcgtgccacaccagctgagcgccagcttgtcttcaatgaaatcctccaggctgcctaccaactcatggtggatgtgtttggtaattacgtcattcagaagttctttgaatttggcagtcttgaacagaagctggctttggcagaacggattcgaggccacgtcctgtcattggcactacagatgtatggctgccgtgttatccagaaagctcttgagtttattccttcagaccagcagaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatccttatggctgccgagtgattcagagaatcctggagcactgtctccctgaccagacactccctattttagaggagcttcaccagcacacagagcagcttgtacaggatcaatatggaaattatgtaatccaacatgtactggagcacggtcgtcctgaggataaaagcaaaattgtagcagaaatccgaggcaatgtacttgtattgagtcagcacaaatttgcaagcaatgttgtggagaagtgtgttactcacgcctcacgtacggagcgcgctgtgctcatcgatgaggtgtgcaccatgaacgacggtccccacagtgccttatacaccatgatgaaggaccagtatgccaactacgtggtccagaagatgattgacgtggcggagccaggccagcggaagatcgtcatgcataagatccggccccacatcgcaactcttcgtaagtacacctatggcaagcacattctggccaagctggagaagtactacatgaagaacggtgttgacttagggcccatctgtggcccccctaatggtatcatctgaggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaagaaaaaaaggaaaaggtttttagctcatttgcctggccggcaagttttgcaaatagctcttccccacctcctcattttagtaaaaaacaaacaaaaacaaaaaaacctgagaagtttgaattgtagttaaatgaccccaaactggcatttaacactgtttataaaaaatatatatatatatatatatatataatgaaaaaggtttcagagttgctaaagcttcagtttgtgacattaagtttatgaaattctaaaaaatgccttttttggagactatattatgctgaagaaggctgttcgtgaggaggagatgcgagcacccagaacgtcttttgaggctgggcgggtgtgattgtttactgcctactggatttttttctattaacattgaaaggtaaaatctgattatttagcatgagaaaaaaaaatccaactctgcttttggtcttgcttctataaatatatagtgtatacttggtgtagactttgcatatatacaaatttgtagtattttcttgttttgatgtctaatctgtatctataatgtaccctagtagtcgaacatacttttgattgtacaattgtacatttgtatacctgtaatgtaaatgtggagaagtttgaatcaacataaacacgttttttggtaagaaaagagaattagccagccctgtgcattcagtgtatattctcaccttttatggtcgtagcatatagtgttgtatattgtaaattgtaatttcaaccagaagtaaatttttttcttttgaaggaataaatgttctttatacagcctagttaatgtttaaaaagaaaaaaatagcttggttttatttgtcatctagtctcaagtatagcgagattctttctaaatgttattcaagattgagttctcactagtgtttttttaatcctaaaaaagtaatgttttgattttgtgacagtcaaaaggacgtgcaaaagtctagccttgcccgagctttccttacaatcagagcccctctcaccttgtaaagtgtgaatcgcccttcccttttgtacagaagatgaactgtattttgcattttgtctacttgtaagtgaatgtaacatactgtcaattttccttgtttgaatatagaattgtaacactacacggtgtacatttccagagccttgtgtatatttccaatgaacttttttgcaagcacacttgtaaccatatgtgtataattaacaaacctgtgtatgcttatgcctgggcaactattttttgtaactcttgtgtagattgtctctaaacaatgtgtgatctttattttgaaaaatacagaactttggaatctgaaaaaaaaaaaaaaaaaaaaaaaaaaaaaaa

SEQ ID NO: 20 - protein, 1186 aa, Homo sapiens; identical to SEQ ID NO: 18

SEQ ID NO: 21 - DNA, 5390 bp, Homo sapiens; identical to SEQ ID NO: 19

SEQ ID NO: 22 - protein, 1186 aa, Homo sapiens; identical to SEQ ID NO: 18

SEQ ID NO: 23 - protein, 156 aa, Homo sapiens
MCHSRSCHPTMTILQAPTPAPSTIPGPRRGSGPEIFTFDPLPEPAAAPAGRPSASRGHRKRSRRVLYPRVVRRQLPVEEPNPAKRLLFLLLTIVFCQILMAEEGVPAPLPPEDAPNAASLAPTPVSPVLEPFNLTSEPSDYALDLSTFLQQHPAAF

SEQ ID NO: 24 - DNA, 697 bp, Homo sapiens
gaattcgcccttcacctttcaattttgtcgcactctccttgtttttgacaatgcaatcatatgcttctgctatgttaagcgtattcaacagcgatgattacagtccagctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtacagggatgactgtacgaaagccacacactcaagaatggagcaccagctaggccacttaccgcccatcaacgcagccagctcaccgcagcaacgggaccgctacagccactggacaaagctgtaggacaatcgggtaacattggcaagggcgaattc

SEQ ID NO: 25 - protein, 204 aa, Homo sapiens
MQSYASAMLSVFNSDDYSPAVQENIPALRRSSSFLCTESCNSKYQCETGENSKGNVQDRVKRPMNAFIVWSRDQRRKMALENPRMRNSEISKQLGYQWKMLTEAEKWPFFQEAQKLQAMHREKYPNYKYRPRRKAKMLPKNCSLLPADPASVLCSEVQLDNRLYRDDCTKATHSRMEHQLGHLPPINAASSPQQRDRYSHWTKL

SEQ ID NO: 26 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 27 - protein, 334 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASK

SEQ ID NO: 28 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

(19) United States
(12) Patent Application Publication
(10) Pub. No.: US 2008/0032293 A1
(43) Pub. Date: Feb. 7, 2008

(54) Housekeeping Genes And Methods For Identifying Same

(75) Inventors: Aniko Szabo, Brookfield, WI (US); Phillip Bernard, Salt Lake City, UT (US); Charles M. Perou, Carrboro, NC (US)

Correspondence Address: NEEDLE & ROSENBERG, P.C., SUITE 1000, 999 PEACHTREE STREET, ATLANTA, GA, 30309-3915, US

(73) Assignees: The University of North Carolina at Chapel Hill, Chapel Hill, NC (US); University of Utah, Salt Lake City, UT (US); University of Utah Research Foundation, Salt Lake City, UT (US)

(21) Appl. No.: 11/632,538

(22) PCT Filed: Jul. 15, 2005
(86) PCT No.: PCT/US05/25105

§ 371 Date: Jul. 13, 2007

Related U.S. Application Data
(60) Provisional application No. 60/588,222, filed on Jul. 15, 2004.

(51) Int. Cl. C12Q 1/68

(52) U.S. Cl. 435/6

(57) ABSTRACT

Disclosed are methods compositions and methods related to housekeeping genes and methods and compositions related to detecting and classifying cancer.

### CROSS REFERENCE TO RELATED APPLICATIONS

This application claims the benefit of U.S. provisional application Ser. No. 60/588,222, filed Jul. 15, 2004. This application is hereby incorporated by this reference in its entirety for all of its teachings.

### ACKNOWLEDGEMENTS

This work was supported in pair by the National Cancer Institute (R33 CA097769-01). The United States Government may have certain rights in the inventions disclosed herein.

### BACKGROUND

There is a need for statistical methods to identify genes that have minimal variation in expression across a variety of experimental conditions. These “housekeeper” genes have application as controls for quantification of test genes using gel analysis and real-time quantitative RT-PCR, for example.

### SUMMARY

Disclosed herein are methods and compositions for identifying housekeeping genes and methods of using the identified housekeeping genes. Real-time quantitative RT-PCR was used to analyze 80 primary breast tumors for variation in expression of 6 putative housekeeper genes (i.e. expression control genes): MRPL19 (SEQ ID NO:1), PSMC4 (SEQ ID NO:2), SF3A1 (SEQ ID NO:3), PUM1 (SEQ ID NO:4), ACTB (SEQ ID NO:5) and GAPD SEQ ID NO:6). Also disclosed are appropriate models for selecting the best housekeepers to normalize quantitative data within a given tissue type (e.g., breast cancer) and across different types of tissue samples.

Disclosed are methods and compositions related to diagnosing cancers, such as breast cancer. Also disclosed are algorithms and methods of using these algorithms related to identifying genes for diagnosing cancer, such as breast cancer.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the expression levels for the five genes shown by tissue sample. Top: raw data. Bottom: log-scale.

FIG. 2 shows the expression levels of the 10 genes shown by sample and tissue type. Vandesompele data set in log-scale.

FIG. 3 shows the mean squared error (MSE) of each gene by tissue-type. The sign is determined by the direction of the bias. The MSE is broken down into the contributing components of the squared bias (Biasˆ2) and the variance (Sigmaˆ2). Vandesompele data set.

FIG. 4 shows two-way hierarchical clustering of microarray data for the same samples assayed by qRT-PCR. Samples were classified based on the expression of 402 “intrinsic” genes defined in Sorlie et al. 2003. The expression level for each gene is shown relative to the median expression of that gene across all the samples with high expression represented by red and low expression represented by green. Genes with median expression are black and missing values are gray. The sample-associated dendrogram shows the same classes seen by qRT-PCR (FIG. 5). Samples are grouped into Luminal, HER2+/ER−, Normal-like, and Basal-like subtypes. Overall, 114/123 (93%) primary breast samples classified the same between microarray and qRT-PCR.

FIG. 5 shows two-way hierarchical clustering of real-time qRT-PCR data from 126 unique samples. The sample-associated dendrogram (5A) shows the same classes seen by microarray. Samples are grouped into Luminal (blue), HER2+/ER− (pink), Normal-like (green), and Basal-like (red) subtypes. The expression level for each gene is shown relative to the median expression of that gene across all the samples with high expression represented by red and low expression represented by green. Genes with median expression are black and missing values are gray. A minimal set of 37 “intrinsic” genes (5B) was used to classify tumors into their primary “intrinsic” subtypes. The “intrinsic” gene set was supplemented using PgR and EGFR (5C), and proliferation genes (5D). The genes in 1C and 1D were clustered separately in order to determine agreement between the minimal 37 qRT-PCR “intrinsic” set (5A) and the larger 402 microarray “intrinsic” set.

FIG. 6 shows Receiver Operator Curves. The agreement between immunohistochemistry (IHC) and gene expression is shown for ER (6A), PR (6B), and HER2 (6C) using ROC. A cut-off for relative gene copy number was selected by minimizing the sum of the observed false positive and false negative errors. The sensitivity and specificity of the resulting classification rule were estimated via bootstrap adjustment for optimism. Since many biomarkers having concordant expression and can serve as surrogates for one another, we tested the accuracy of using GATA3 and GRB7 as surrogates (dotted lines) for calling ER and HER2 protein status, respectively. There was overall good agreement between gene expression and IHC status for ER and PR, but poor agreement between gene expression and IHC status for HER2. The surrogate markers had similar accuracy to the actual markers for predicting IHC status.

FIG. 7 shows outcome for “intrinsic” subtypes. Kaplan-Meier plots showing relapse free survival (RFS) and overall survival (OS) for patients with Luminal tumors compared to those with HER2+/ER− or Basal-like tumors. Patients with Luminal tumors showed significantly better outcomes for RFS (3A) and OS (3B) compared to HER2+/ER− (RFS: p=0.023; OS: p=0.003) and Basal-like (RFS: p=0.065; OS: p=0.002) tumors. Classifications were made from real-time qRT-PCR data using the minimal 37 “intrinsic” gene list. Pairwise log-rank tests were used to test for equality of the hazard functions among the intrinsic classes. Tumors in the Normal Breast-like subtype were excluded from the analyses since this class may be artificially created from having a sample comprised primarily of normal cells.

FIG. 8 shows grade and proliferation as predictors of relapse free survival. Kaplan-Meier plots are shown for grade (8A) and the proliferation genes (8B) using Cox regression analysis. The analysis for the proliferation genes was performed on continuous expression data, although the plots are shown in tertiles. The proliferation index (log average of the 14 proliferation genes) has significant predictive value for outcome, even after correcting for other clinical parameters important for survival. Furthermore, when we include both grade and the proliferation index (and stage) in a model for RFS, we find that the proliferation index is the superior predictor (Grade p=0.51; Proliferation index p=0.047).

FIG. 9 shows co-clustering of real-time qRT-PCR and microarray data using 50 genes and 252 samples. The relative copy number (qRT-PCR) and R/G ratio (microarray) for each gene was log2 transformed and combined into a single dataset using distance weighted discrimination. Two-way hierarchical clustering was performed on the combined dataset using Spearman correlation and average linkage. The sample associated dendrogram (5A) shows the same classes as seen in FIG. 1. Samples are classified as Basal-like (red), HER2+/ER−, Luminal, and Normal-like. The expression level for each gene is shown relative to the median expression of that gene across all the samples with overexpressed genes and underexpressed genes, as well as average expression. The gene associated dendrogram (5B) shows that the Luminal tumors and Basal-like tumors differentially express estrogen associated genes (cluster 1); as well as basal keratins (KRT 5 and 17), inflammatory response genes (CX3CL1 and SLPI), and genes in the Wnt pathway (FZD7) (cluster 3). The main distinguishers of the HER2+/ER− group are low expression of genes in cluster 1 and high expression of genes on the 17q12 amplicon (ERBB2 and GRB7) (cluster 4). The proliferation genes (cluster 2) have high expression in the ER negative tumors (Basal-like and HER2+/ER−) and low expression in ER positive (Luminal) and Normal-like samples.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. COMPOSITIONS AND METHODS

Genes that exhibit minimal variation in messenger RNA (mRNA) quantity across a variety of cell types and biological conditions provide valuable controls for relative quantification. Normalizing quantitative data with housekeeper(s) or controls has many applications from identifying genes regulated during embryogenesis to developing new cancer diagnostics. Although finding biological significance in gene expression data can rely heavily on the performance of the housekeeper genes or expression control genes, there is a paucity of information on testing these genes for their suitability.

The copy number of a housekeeper gene or expression control genes should be proportional to the amount of polyA RNA present in sample and this proportion should be maintained across a variety of experimental conditions. Since nucleic acids show high absorbance at 260 nm (A260), spectrophotometers provide approximate amounts of total DNA/RNA present in a sample. Using absorbance methods alone, however, gives no information about the type of nucleic acid (e.g., DNA versus RNA) or contributions from different nucleic acid fractions (e.g., rRNA versus mRNA). It can be assumed that mRNA comprises approximately 1-3% of the total RNA. However, this contribution may change depending on the extraction method used. For instance, column extraction methods provide better exclusion of ribosomal RNA than using solvent extraction methods (Miller C L, Yolken R H, Brain Res Brain Res Protoc 2003, 10:156-167). By combining capillary electrophoresis with absorbance, it is possible to accurately quantify these different fractions (Panaro N J, et al., Clin Chem 2000, 46:1851-1853).

Relative quantification by Northern blot analysis has traditionally used housekeepers or expression controls to represent the amount of mRNA in the sample and to control for sample loading, blot transfer and probe hybridization. Highly expressed genes serving fundamental roles in the cell, such as GAPD, β-actin (ACTB), and ribosomal proteins, are commonly used for this purpose but, as disclosed and shown herein, are not optimal under certain experimental conditions (Suzuki T, et al., Biotechiniques 2000, 29:332-337); Bhatia P, et al., Anal Biochem 1994, 216:223-226; (Spanakis E., Nucleic Acids Res 1993, 21:3809-3819). For example, the sensitivity and accuracy of Northern blot analysis with densitometry can be decreased using a highly expressed housekeeper gene or expression control gene that can saturate the autoradiographic signal (Eggert A, et al., Biotechniques 2000, 28:681-682, 686, 688-691). To resolve this problem and compensate for limitations in dynamic range, control genes can be chosen to have a level of gene expression similar to the gene(s) of interest (i.e., test genes).

Microarrays are more practical for genome-wide expression analysis than Northern blots (Schena M, et al., Science 1995, 270:467-470). With cDNA microarrays, a common reference sample is usually used to compare the expression of each gene across many experimental sample(s) (Peron C M, et al., Nature 2000, 406:747-752; van de Vijver M J, et al., N Engl J Med 2002, 347:1999-2009). Since each gene in the experimental sample is directly compared to the same gene in the common reference, housekeeper genes or expression control genes are not necessary for normalization. Microarrays are commonly applied to finding genes with differential expression across experimental conditions but the data may also be used to identify stably expressed genes that can serve as important controls for Northern blot analysis, ribonuclease protection assays, and quantitative RT-PCR. In turn, these other quantitative methods are often used to verify differentially expressed genes identified by microarray (Dhanasekaran S M, et al., Nature 2001, 412:822-826; Welsh J B, et al., Proc Natl Acad Sci USA 2001, 98:1176-1181; (Mischel P S, et al., Cancer Biol Ther 2003, 2:242-247).

Housekeeper genes or expression control genes are often adopted from the literature and used across a variety of experimental conditions, some of which may induce differences in their expression. If unrecognized, unexpected changes in housekeeper expression could result in erroneous conclusions about real biological effects (e.g., drug response). In addition, this type of change would be difficult to detect because most experiments only include a single housekeeper gene or expression control gene. It is difficult to determine whether a given gene has the constitutive property of a housekeeper when the true amount of mRNA in a sample is unknown. As a way around this dilemma, Vandesompele et al postulated that gene pairs that have stable expression patterns relative to each other are proper control genes (Vandesompele J, et al., Genome Biol 2002, 3:RESEARCH0034). An alternative method for quantitative analysis of RT-PCR data that does not require housekeeper genes or expression control genes for normalization is using global pattern recognition (GPR). For instance, Akilesh et al. used a GPR algorithm to search for eligible normalizing genes within an assay plate and then used those genes as controls to identify differentially expressed genes (Akilesh S, et al., Genome Res 2003, 13:1719-1727). Although relative quantification with housekeeper genes or expression control genes is a practical method to estimate the expression level of a test gene, the transcript amount in the sample is a summation, and the method does not consider transcript differences on a cell-to-cell basis. Fluorescence in-situ hybridization (FISH) is clinically used to determine absolute DNA copy number (e.g., HER2 amplification) in a cell but these methods still average the copy number after counting many cells and the technique is expensive and laborious (Tubbs R R, et al., J Clin Oncol 2001, 19:2714-2721). In-situ methods for detecting RNA transcripts have been developed but the assays are semi-quantitative and subjective (Kristt D, et al., Pathiol Oncol Res 2000, 6:65-70).

Disclosed herein are models and methods for selecting the best housekeeper genes or expression control genes for breast cancer, as well as algorithms to be used in methods that can be generalized to find housekeeper genes or expression control genes that are appropriate for normalizing quantitative data within and between tissue types.

Disclosed herein are methods where one expression control gene is MRPL19 (SEQ ID NO:1). Disclosed are methods using this expression control gene and others disclosed herein as controls for sample quality and for PCR in assays that test for abnormalities in cancer, such as translocations, such as translocations in sarcomas.

1. Genes as Housekeepers for Cancer

A housekeeper gene is a gene that has minimal variation across DNA samples, making it good for use as a control when assaying expression of other genes across sample. No gene has absolute homeostasis across all tissues or samples. Disclosed herein are expression control genes that can be used as housekeeper genes are used. The expression control genes disclosed herein can be genes that have less than or equal to 0.1, 0.2. 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% variation between two different tissues. It is also understood that these levels of variation can also be applied across 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more tissues. It is also understood that variation can be determined as discussed in the examples using the algorithms as disclosed herein.

There are a variety of different genes which can be used as expression control genes, alone or in combination. For example, MRPL19 (SEQ ID NO:1), PSMC4 (SEQ ID NO:2), SF3A1 (SEQ ID NO:3), PUM1 (SEQ ID NO:4), ACTB (SEQ ID NO:5) and GAPD (SEQ ID NO:6) are genes that can be expression control genes. Other genes as disclosed herein can also be considered expression control genes, such as the sequences set forth in the SEQ ID NOs 1-27.

The expression control genes can be used in any combination or singularly in any method described herein. It is also understood that any nucleic acid related to the expression control genes, such as the RNA, mRNA, exons, introns, or 5′ or 3′ upstream or downstream sequence, or DNA or gene can be used or identified in any of the methods or with any of the compositions disclosed herein.

2. Molecules for Detecting Genes, Gene Expression Products, Proteins Encoded by Genes

The disclosed methods involve using specific housekeeper genes or gene sets or expression control genes or gene sets such that they are detected in some way or their expression product is detected in some way. Typically the expression control gene or its expression product will be detected by a primer or probe as disclosed herein. However, it is understood that they can also be detected by any means, such as a specific monoclonal antibody or other visualization technique. Often, the expression control genes or housekeeper genes or their expression products can be detected after or through some amplification process, such as RT-PCR, including quantitative PCR.

a) Primers and Probes

It is understood that primers and probes can be produced for the actual gene (DNA) or expression product (mRNA) or intermediate expression products which are not fully processed into mRNA. Discussion of a particular gene, such as MRPL19 (SEQ ID NO:1) is also a disclosure of the DNA, mRNA, and intermediate RNA products associated with that particular gene.

Disclosed are compositions including primers and probes, which are capable of interacting with the MRPL19 (SEQ ID NO:1), PSMC4 (SEQ ID NO:2), SF3A1 (SEQ ID NO:3), and PUM1 (SEQ ID NO:4) genes as wells those disclosed herein, as well as the any other genes or nucleic acids discussed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed genes or regions of the disclosed genes or they hybridize with the complement of the disclosed genes or complement of a region of the disclosed genes.

The size of the primers or probes for interaction with the disclosed genes in certain embodiments can be any size that supports the desired enzymatic manipulation of the primer, such as DNA amplification or the simple hybridization of the probe or primer. A typical disclosed primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the disclosed primers or probes can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the disclosed genes in certain embodiments can be used to produce an amplified DNA product that contains the desired region of the disclosed genes. In general, typically the size of the product will be such that the size can be accurately determined to within 10, 5, 4, 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In certain embodiments the primers and probes are designed such that they are targeting as specific region in one of the genes disclosed herein. It is understood that primers and probes having an interaction with any region of any gene disclosed herein are contemplated. In other words, primers and probes of any size disclosed herein can be used to target any region specifically defined by the genes disclosed herein. Thus, primers and probes of any size can begin hybridizing with nucleotide 1, 2, 3, 4, 5, 6, 7, 8, 9, 10, or any specific nucleotide of the genes or gene expression products disclosed herein. Furthermore, it is understood that the primers and probes can be of a contiguous nature meaning that they have continuous base pairing with the target nucleic acid for which they are complementary. However, also disclosed are primers and probes which are not contiguous with their target complementary sequence. Disclosed are primers and probes which have at least 1, 2, 3, 4, 5, 6, 7, 8, 9, 10, 15, 20, 25, 30, 35, 40, 50, 75, 100, 150, 200, 500, or more bases which are not contiguous across the length of the primer or probe. Also disclosed are primers and probes which have less than or equal to 1, 2, 3, 4, 5, 6, 7, 8, 9, 10, 15, 20, 25, 30, 35, 40, 50, 75, 100, 150, 200, 500, or more bases which are not contiguous across the length of the primer or probe.

In certain embodiments the primers or probes are designed such that they are able to hybridize specifically with a target nucleic acid. Specific hybridization refers to the ability to bind a particular nucleic acid or set of nucleic acids preferentially over other nucleic acids. The level of specific hybridization of a particular probe or primer with a target nucleic acid can be affected by salt conditions, buffer conditions, temperature, length of time of hybridization, wash conditions, and visualization conditions. By increasing the specificity of hybridization means decreasing the number of nucleic acids that a given primer or probe hybridizes to typically under a given set of conditions. For example, at 20 degrees Celsius under a given set of conditions a given probe may hybridize with 10 nucleic acids in a sample. However, at 40 degrees Celsius with all other conditions being equal, the same probe may only hybridize with 2 nucleic acids in the same sample. This would be considered an increase in specificity of hybridization. A decrease in specificity of hybridization means an increase in the number of nucleic acids that a given primer or probe hybridizes to typically under a given set of conditions. For example, at 700 mM NaCl under a given set of conditions a particular probe or primer may hybridize with 2 nucleic acids in a sample, however when the salt concentration is increased to 1 Molar NaCl the primer or probe may hybridize with 6 nucleic acids in the same sample.

The salt can be any salt such as those made from the alkali metals: Lithium, Sodium, Potassium, Rubidium, Cesium, or Francium or the alkaline earth metals: Beryllium, Magnesium, Calcium, Strontium, Barium, or Radiumsodium, or the transition metals: Scandium, Titanium, Vanadium, Chromium, Manganese, Iron, Cobalt, Nickel, Copper, Zinc, Yttrium, Zirconium, Niobium, Molybdenum, Technetium, Ruthenium, Rhodium, Palladium, Silver, Cadmium, Hafnium, Tantalum, Tungsten, Rhenium, Osmium, Iridium, Platinum, Gold, Mercury, Rutherfordium, Dubnium, Seaborgium, Bohrium, Hassium, Meitnerium, Ununnilium, Unununium or Ununbium at any molar strength to promoter the desired condition, such as 1, 0.7, 0.5, 0.3, 0.2, 0.1, 0.05, or 0.02 molar salt. In general increasing salt concentration decreases the specificity of a given probe or primer for a given target nucleic acid and decreasing the salt concentration increases the specificity of a given probe or primer for a given target nucleic acid.

The buffer conditions can be any buffer such as TRIS at any pH, such as 5.0, 5.5, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.5, or 9.0. In general pHs above or below 7.0 increase the specificity of hybridization.

The temperature of hybridization can be any temperature. For example, the temperature of hybridization can occur at 20°, 21°, 22°, 23°, 24°, 25°, 26°, 27°, 28°, 29°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 81°, 82°, 83°, 84°, 85°, 86°, 87°, 88°, 89°, 90°, 91°, 92°, 93°, 94°, 95°, 96°, 97°, 98°, or 99° Celsius.

The length of time of hybridization can be for any time. For example, the length of time can be for 1, 2, 3, 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 150, 180, 210, 240, 270, 300, 360, minutes or 7, 8, 9, 10, 11, 12, 13, 14, 15, 16, 17, 18, 19, 20, 21, 22, 23, 24, 30, 36, 48 or more hours.

It is understood that any wash conditions can be used including no wash step. Generally the wash conditions occur by a change in one or more of the other conditions designed to require more specific binding, by for example increasing temperature or decreasing the salt or changing the length of time of hybridization.

It is understood that there are a variety of visualization conditions which have different levels of detection capabilities. In general any type of visualization or detection system can be used. For example, radiolabeling or fluorescence labeling can be used and in general fluorescence labeling would be more sensitive, meaning a fewer number of absolute molecules would have to be present to be detected.

3. Method of Diagnosing or Prognosing Cancer

Microarrays have shown that gene expression patterns can be used to molecularly classify various types of cancers into distinct and clinically significant groups. In order to translate these profiles into routine diagnostics, a microarray breast cancer classification system has been recapitulated using real-time quantitative (q)RT-PCR (Example 2). Statistical analyses were performed on multiple independent microarray datasets to select an “intrinsic” gene set of 550 genes that can classify breast tumors into four different subtypes designated as Luminal, Normal-like, HER2+/ER−, and Basal-like. Intrinsic genes, as described in Perou et al. (2000) Nature 406:747-752, are statistically selected to have low variation in expression between biological sample replicates from the same individual and high variation in expression across samples from different individuals. Thus, “intrinsic genes” are the classifier (or experimental) genes for breast cancer classification and each classifier gene must be normalized to the housekeeper (or control) genes in order to make the classification. A minimal gene set from the microarray “intrinsic” list, and additional genes important for outcome (e.g., proliferation genes), were used to develop a real-time qRT-PCR assay comprised of 53 classifiers and 3 housekeepers. The expression data and classifications from microarray and real-time qRT-PCR were respectively compared using 123 unique breast samples (117 invasive carcinomas, 1 tibroadenoma and 5 normal tissues) and 3 cells lines. The overall correlation for the 50 genes in common between microarray and qRT-PCR was 0.76. There was 91% (114/126) concordance in the hierarchical clustering classification of the real-time qRT-PCR minimal “intrinsic” gene set (37 genes) and the larger (550 genes) microarray gene set from which the PCR list was derived. As expected, the Luminal tumors (ER+) had a significantly better outcome than the HER2+/ER− (p=0.043) and Basal-like tumors (p=0.001). High expression of the proliferation genes GTBP4 (p=0.011), HSPA14 (p=0.023), and STK6 (p=0.027) were significant predictors of relapse free survival (RFS) independent of grade and stage. This study shows that genomic microarray data can be translated into a qRT-PCR diagnostic assay that would improve the standard of care in breast cancer.

A major challenge in the clinical care of cancer has been providing an accurate diagnosis for appropriate management of the disease. For over 50 years, medicine has relied on morphological features (histopathology) and anatomic staging (Tumor size/Node involvement/Metastasis) for classification of tumors (Greenough, R. B. J Cancer Res 9:452-463; Bloom et al. (1957) British Journal of Cancer 9:359-377). The TNM staging system provides information about the extent of disease and has been the “gold standard” for prognosis benson, et al. (1991) Cancer 68:2142-2149; Fitzgibbons, et al (2000) Arch Pathol Lab Med 124:966-978).

In addition to TNM, the grade of the tumor is also prognostic for relapse free survival (RFS) and overall survival (OS) (Elston et al. (1991) Histopathology 19:403-410). Tumor grade is determined from histological assessment of tubule formation, nuclear pleomorphism, and mitotic count. Due to the subjective nature of grading and difficulties standardizing methods, there has been less than optimal agreement between pathologists (Dalton et al. (1994) Cancer 73:2765-2770). Applying the Nottingham combined histological grade has made scoring more quantitative and improved agreement between observers (Frierson (1995) Am J Clin Pathol 103:195-198), however, more objective methods are still needed before grade is integrated into the TNM classification (Singletary (2003) Surg Clin North Am 83:803-819). For instance, most studies show significance in outcome between Grade 1 (low/least aggressive) and Grade 3 (high/most aggressive), but Grade 2 (intermediate) tumors show variability in outcome and are commonly not classified the same across institutions (Kollias et al. (1999) Eur J Cancer 35:908-912; Robbins et al. (1995) Hum Pathol 26:873-879; Genestie et al. (1998) Anticancer Res 18:571-576.). Alternatively, proliferation assays, such as S-phase fraction and mitotic index, have shown to be independent prognostic indicators and could be used in conjunction with, or instead of grade (Michels et al. (2004) Cancer 100:455-464; Caly et al. (2004) Anticancer Res 24:3283-3288).

Women with the same stage of breast cancer can have widely different clinical outcomes due to differences in tumor biology (van't Veer et al. (2002) Nature 415:530-536; van de Vijver et al. (2002) N Engl J Med 347:1999-2009). The use of gene expression markers in breast pathology can provide addition clinical information that complements the TNM system for prognosis and is important for making therapeutic decisions (van't Veer et al. (2002) Nature 415:530-536; van de Vijver et al. (2002) N Engl J Med 347:1999-2009; Paik et al. (2004) N Engl Med 351:2817-2826; Sorlie et al. (2001) Proc Natl Acad Sci USA 98:10869-10874; Sorlie et al. (2003) Proc Natl Acad Sci USA 100:8418-8423). Undoubtedly, one of the greatest advancements in breast cancer medicine has been the identification and routine testing for the expression of the hormone receptors, namely the Estrogen Receptor (ER) and the Progesterone Receptor (PgR), which allows the clinician to offer endocrine blockade therapy that can significantly prolong survival in women with tumors expressing these proteins (Buzdar et al. (2003) J Clin Oncol 21:1007-1014; Fisher et al (1989) N Engl J Med 320:479-484).

Although ER expression is a predictive marker, it also serves as a surrogate marker for describing a tumor biology that is characteristically less aggressive (e.g. lower grade) than ER-negative tumors (Fisher et al. (1981) Breast Cancer Res Treat 1:37-41). Microarrays have elucidated the richness and diversity in the biology of breast cancer and have identified many genes that associate with ER-positive and ER-negative tumors Perou et al. (2000) Nature 406:747-752; West et al. (2001) Proc Natl Acad Sci USA 98:11462-11467; Gruvberger et al. (2001) Cancer Res 61:5979-5984). When microarray data from invasive breast carcinomas are analyzed by hierarchical clustering, samples are separated primarily based on ER status (Sotiriou et al. (2003) Proc Natl Acad Sci USA 100:10393-10398).

One method for characterizing the diverse biology that exists across breast cancer is analysis of an “intrinsic” gene set comprised of genes that vary in expression between tumors from different individuals but have little variation in expression between replicates from the same individual. Perou et al. found that an intrinsic gene set derived from before and after chemotherapy tumor pairs could be used to classify breast cancer into at least 4 groups: Luminal, Normal-like, HER2+/ER−, and Basal-like. Additional studies using larger patient sets have shown that these subtypes can be identified in independent data sets, and always make the same prognostic outcome predictions (Yu et al. (2004) Clin Cancer Res 10:5508-5517).

Breast tumors of the “Luminal” subtype are ER positive and have a similar keratin expression profile as the epithelial cells lining the lumen of the breast ducts (Taylor-Papadimitriou et al. (1989) J Cell Sci 94:403-413; Peron et al. (2000) New Technologies for life sciences: A Trends Guide: 67-76). Conversely, ER-negative tumors can be broken into two main subtypes, namely those that overexpress (and are DNA amplified for) HER2 and GRB7 (HER2+/ER−), and “Basal-like” tumors that have an expression profile similar to basal epithelium and express Keratin 5, 6B and 17. Both these tumor subtypes are aggressive and typically more deadly than Luminal tumors; however, there are subtypes of Luminal tumors that lead to poor outcome despite being ER-positive. For instance, Sorlie et al. identified a Luminal B subtype with similar outcomes to the HER2+/ER− and Basal-like subtypes, and Sotiriou et al. showed that there are 3 different types of Luminal tumors with different outcomes. The Luminal tumors with poor outcomes consistently share the histopathological feature of being higher grade and the molecular feature of highly expressing proliferation genes.

The so called “proliferation genes” show periodicity in expression through the cell cycle and have a variety of functions necessary for cell growth, DNA replication, and mitosis (Whitfield et al. (2002) Mol Biol Cell 13:1977-2000; Ishida et al. Mol Cell Biol 21:4684-4699). Despite their diverse functions, proliferation genes have similar gene expression profiles when analyzed by hierarchical clustering. As might be expected, proliferation genes correlate with grade, the mitotic index (Perou et al. (1999) Proc Natl Acad Sci USA 96:9212-9217), and outcome (Sorlie et al. (2001) Proc Natl Acad Sci USA 98:10869-10874). Proliferation genes are often selected when supervised analysis is used to find genes that correlate with patient outcome. For example, the SAM264 “survival” list presented in Sorlie et al., the 231 “prognosis classifier” list in van't Veer et al., and the “485 prognostic gene” list in Sotiriou et al., identified common proliferation genes (PCNA, TOP2A, CENPF). This suggests that all these studies are likely tracking a similar phenotype.

Gene expression profiling using DNA microarrays is a powerful tool to discover genes for molecular classifications of cancer but the platforms are labor intensive, expensive and currently not amenable to routine clinical diagnostics. Real-time qRT-PCR is well-suited for solid tumor diagnostics since it is rapid, homogenous (amplification and quantification in a single vessel), and can be performed from archived (FFPE tissue) samples. It has been shown that “intrinsic” breast cancer classifications from microarray can be recapitulated by qRT-PCR using a minimal “intrinsic” gene set. In addition, by supplementing the “intrinsic” gene set with proliferation genes, a more objective measurement of grade has been developed. The assay disclosed herein adds prognostic information to the standard of care for breast cancer.

Microarray used in conjunction with RT-PCR provides a powerful system for discovering and translating genomic markers into the clinical laboratory for molecular diagnostics. Although these platforms are fundamentally very different, the quantitative data across the methods have a high correlation. In fact, the data across the methods is no more disparate then across different microarray platforms. By hierarchical clustering, it has been shown that a biological classification of breast cancer derived from microarray data can be recapitulated using real-time qRT-PCR. Biological classification by real-time qRT-PCR makes the important clinical distinction between ER positive and ER negative tumors and identifies additional subtypes that have prognostic and predictive value.

The benefit of using real-time qRT-PCR for cancer diagnostics is that new informative markers can be readily validated and implemented, making tests expandable and/or tailored to the individual. For instance, it has been shown that including proliferation genes serves a similar purpose to grade but is more prognostic. Since grade has been shown to be universal as a prognostic factor in cancer, it is likely that the same markers correlate to grade and are important for survival in other tumor types. Real-time qRT-PCR is attractive for clinical use because it is fast, reproducible, tissue sparing, and able to be automated. Although genomic profiling should currently be used for ancillary testing, the fact that normal tissues can be distinguished from tumor tissue shows that these molecular assays may eventually be used for cancer diagnostics without histological corroboration.

Disclosed herein are methods of classifying cancer in a subject, comprising: a) identifying intrinsic genes of the subject to be used to classify the cancer; b) obtaining a sample from the subject; c) amplifying and detecting levels of intrinsic genes in the subject; and d) classifying cancer based upon results of step c.

Also disclosed are methods of prognosing the survival of a subject, comprising using the methods disclosed herein to detect intrinsic gene expression in a subject, and classifying the type of tumor based upon that information, thereby prognosing the survival of subject based on the outcome of the tumor classification. The methods disclosed herein can be used with any of the types of cancer listed herein. The cancer can be breast cancer, for example. The breast cancer can be classified into one of four groups: luminal, normal-like, HER2+/ER− and basal-like, for example.

Disclosed are compositions and methods which can be used in quantitation of target nucleic acids, such as the expression levels of genes involved in cancer, such as breast cancer, such as HER2. The method includes using housekeeping genes or expression control genes to normalize for differences in sample input and/or differences in PCR or pre-PCR reaction efficiencies.

Disclosed are methods of quantifying the amount of nucleic acid in a sample, such as a standard, comprising assaying the amount of expression of one or more of the genes disclosed herein in any combination, using any method. This type of method can be used in conjunction with other assay methods, as for example, a control. For example, disclosed are methods, wherein the expression of one or more of the genes, such as MPRL19 (SEQ ID NO:1, disclosed herein) is assayed during a diagnostic or prognostic test for a sarcoma.

Disclosed are methods comprising comparing the expression of an expression control gene or genes in a first sample to the expression of the expression control gene or genes in a second sample. It is understood that determining the expression of the expression control gene can be performed in any way, including the methods disclosed herein, for example, by RT-PCR with the use of primers as discussed herein, or through hybridization of a probe through for example blotting or array technology.

Also disclosed are methods where the expression levels of a target nucleic acid(s) is compared to the level of expression of one or more expression control genes. A target nucleic acid can be any nucleic acid, such as a test gene, for which data is desired, such as a nucleic acid involved in cancer diagnosis or prognosis, such as HER2.

Disclosed are methods of analyzing nucleic acid expression levels in a sample, the methods comprising comparing expression levels of a housekeeping gene or expression control gene to a test nucleic acid, wherein elevated expression of the test gene relative to the housekeeping gene or expression controlling gene indicates a diagnoses, poor prognosis, likelihood of obtaining, predisposition to obtaining, or presence of a cancer. Also disclosed are methods wherein the step of comparing comprises identifying the expression levels of a housekeeping gene or expression control gene and test gene by interaction with a primer or probe.

Disclosed are methods where an elevated expression of a test nucleic acid relative to the housekeeping gene or expression control gene indicates the presence of a cancer, a poor prognosis for a patient having a cancer, a predisposition of getting a cancer, or a diagnoses of cancer or a cancerous state.

Disclosed are methods for quantifying or assaying the expression of a nucleic acid comprising 1) assaying the level of a housekeeping gene or expression control gene in a control sample, 2) assaying the expression of a test gene in the control sample, 3) assaying the amount of the housekeeping gene or expression control gene in a target sample, 4) assaying the expression of the test gene in the target sample, and 5) comparing the amount of expression of the test gene in the control sample to the amount of expression of the test gene in the target sample.

Disclosed are methods wherein the expression of the housekeeping gene or expression control gene and the test gene are compared between a control sample and a target sample. In certain embodiments the assay involves determining if the difference in expression levels between the control sample and the target sample of the test gene is a greater, equal, or lesser difference than the difference between the housekeeping gene or expression control gene between the control sample and the target sample.

In other embodiments the assay involves determining if the amount of the expression of the housekeeping gene or expression control gene has changed less than 0.1, 0.2, 0.3, 0.4, 0.5, 0.6, 0.7, 0.8, 0.9, 1, 2, 3, 4, 5, 6, 7, 8, 9, 10, 11, 12 13, 14, 15, 16, 17, 18, 19, or 20% between the control sample and the target sample.

It is also understood that these changes of both the housekeeping genes or expression control genes and the test genes can also be compared to the expression level of a reference sample which was not tested or obtained at the time of the target sample. This reference sample could be or have been obtained for example by looking at the expression levels of a given gene over many samples and averaging the amount. Those of skill understand how to create new reference samples and how to use existing reference samples.

In the certain assays a determination of whether the housekeeping gene or expression control gene are within a window of tolerance can be done. A window of tolerance is defined as the acceptable amount of variation in expression between two or more samples of the housekeeping gene or expression control gene. For example, the variation can be defined as less than +/−0.1, 0.2, 0.3, 0.4, 0.5, 0.6, 0.7, 0.8, 0.9, 1, 2, 3, 4, 5, 6, 7 8, 9, 10, 11, 12, 13, 14, 15, 16, 17, 18, 19, or 20%.

It is understood that any method of assaying any gene discussed herein can be performed. For example methods of assaying gene copy number or mRNA expression copy number can be performed. For example, RT-PCR, PCR, quantitative PCR, and any other forms of nucleic acid amplification can be performed. Furthermore, methods of hybridization, such as blotting, such as Northern or Southern techniques, such as chip and microarray techniques and any other techniques involving hybridizing of nucleic acids.

Disclosed are methods of quantitating level of expression of a test nucleic acid comprising: a) comparing gene expression levels of a housekeeping gene or expression control gene to a test nucleic acid; and b) quantitating level of expression of the test nucleic acid.

Disclosed are methods of comparing expression levels of the same test nucleic acid expressed in multiple samples, comprising: a) co-amplifying a housekeeping gene or expression control gene and the test nucleic acid; b) normalizing expression of the test nucleic acid amplified in each sample by i) comparing amplification of the housekeeping gene or expression control gene, and ii) applying normalization to the test nucleic acids; and c) comparing expression levels of the test nucleic acids across samples.

Also disclosed are methods of determining a total amount of mRNA in a sample comprising a) measuring expression level of a nucleic acid comprising a housekeeper gene or genes; b) comparing the expression level of the nucleic acid comprising the housekeeper gene to known values for percent of the nucleic acid comprising the housekeeper gene of the total amount of mRNA; c) extrapolating the expression level of the nucleic acid comprising the housekeeper gene to the total amount of mRNA; and d) determining the total amount of mRNA in the sample.

Also disclosed are methods of normalizing the amount of mRNA amplified in multiple samples comprising a) comparing expression levels of a nucleic acid comprising a housekeeper gene across multiple samples; b) deriving a value for normalizing expression of the nucleic acid comprising the housekeeper gene across the multiple samples; and c) normalizing the expression of other nucleic acids amplified in the multiple samples based on the value obtained in step b).

Also disclosed is a method of diagnosing cancer in a subject comprising: a) using a nucleic acid comprising a housekeeper gene as a control; b) amplifying a sample comprising a nucleic acid indicative of cancer; c) determining if the control was amplified at an expected level, and if so, then d) determining if the nucleic acid indicative of cancer was also amplified, and if so then e) diagnosing cancer in the subject.

The selected housekeeper genes, as described in Szabo et al. (2004) Genome Biol 5:R59, have been validated by showing successful application in a pre-clinical real-time qRT-PCR assays important for prognosis in breast cancer. The arithmetic mean of the log expression for the top 3 control genes (MRPL19, PSMC4, PUM1) were used to normalize gene expression for a select group of classifier genes that included an “intrinsic” gene set and proliferation genes. One, or a combination, of the selected housekeepers (Table 10) has clinical utility in developing and using real-time qRT-PCR for molecular diagnostic assays comprised of a single or multiple classifier genes. It has been shown that the housekeepers, together with any single or set of classifiers, can be used in stand alone assays for determining ER status, intrinsic classification, and/or proliferation in breast cancer.

4. A Non-Limiting List of Cancers which can be Assayed with Disclosed Compositions and Methods

The disclosed compositions can be used to diagnose or prognose any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to diagnose or prognose is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the diagnosis or prognosis of precancer conditions such as cervical and anal dysplasias, other dysplasias, severe dysplasias, hyperplasias, atypical hyperplasias, and neoplasias.

5. Methods of Identifying Housekeeping or Expression Control Genes

Disclosed are methods of identifying housekeeper genes or expression control genes from microarrays or other high density nucleic acid samples. The methods generally comprise hybridizing a target sample on a microarray or other high density nucleic acid device and filtering the hybridized sample for a certain level of expression or identification on the microarray. This filtering step in some embodiments involves identifying genes having at least a certain amount of expression, for example Cy3 and Cy5 signal intensities greater than 500 units across at least 75% of the samples. Genes having greater than 50, 100, 150, 200, 250, 300, 350, 400, 450, 550, 600, 650, 700, 750, 800, 900, 1000, 1100, 1200, 1300, 1400, 1500, 1600, 1700, 1800, 1900, 2000, 2500, 3000, 3500, 4000, 4500, and 5000 units of intensities can also be selected. It is also understood that the samples can have these varying levels of intensity across at least 40%, 45%, 50%, 555%, 60%, 65%, 70%, 75%, 80%, 85%, 90%, 95%, 96%, 97%, 98%, 99%, or 100% of the samples tested. One can also filter for nucleic acids having less than a certain amount of expression.

The methods also generally include the step of identifying a gene or set of genes that have a desired level of expression across the samples as discussed herein. The levels of expression can be analyzed using any software including SAS/STAT Analysis Package Version 8 (SAS Institute Inc., Cary, N.C.). Any expression level analysis software can be used. Genes having any of the expression properties of housekeeper genes or expression control genes as discussed herein can be identified.

Disclosed are methods of selecting a best housekeeper gene or expression control gene for a particular tissue comprising: a) obtaining expression data for a set of genes from a sample b) comparing the expression of each gene using the equation: log yᵢⱼ=μ+Tᵢ+Gⱼ+εᵢⱼ, wherein log(yᵢⱼ) represents an expression component for a gene j in sample i, and wherein μ denotes the overall mean (log-) expression, Tᵢ is the difference of the ith tissue sample from the overall average and Gⱼ is the difference of the jth gene from the overall average, wherein Σᵢ₌₁ⁿTᵢ=0, Σⱼ₌₁^{g}Gⱼ=0, wherein εᵢⱼ˜N(0,σⱼ²), and wherein σⱼ is standard deviation; c) identifying a best gene within the set of genes having the lowest standard deviation, σⱼ, wherein the best gene represents the best housekeeper gene or expression control gene for the tissue.

a) Methods for Identifying the Best Housekeeper Gene or Expression Control Gene for a Specific Tissue

Disclosed are methods of selecting a best housekeeper gene or expression control gene for a particular tissue comprising: a) obtaining expression data for a set of genes from a sample b) comparing the expression of each gene using the equation: log yᵢⱼ=μ+Tᵢ+Gⱼ+εᵢⱼ, wherein log(yᵢⱼ) represents an expression component for a gene j in sample i, and wherein i denotes the overall mean (log-) expression, Tᵢ is the difference of the ith tissue sample from the overall average and Gⱼ is the difference of the jth gene from the overall average, wherein Σᵢ₌₁ⁿTᵢ=0, Σⱼ₌₁^{g}Gⱼ=0, wherein εᵢⱼ˜N(0,σ²) and wherein σⱼ is standard deviation; c) identifying a best gene within the set of genes having the lowest standard deviation, σⱼ, wherein the best gene represents the best housekeeper gene or expression control gene for the tissue.

b) Methods for Identifying the Best Housekeeper Gene or Expression Control Gene

Disclosed are methods of selecting a best housekeeper gene or expression control gene for a particular tissue comprising: a) obtaining expression data for a set of genes from a sample b) comparing the expression of each gene using the equation: log yᵢⱼ=μ+Tᵢ+Gⱼ+εᵢⱼ, wherein log(yᵢⱼ) represents an expression component for a gene j in sample i, and wherein μ denotes the overall mean (log-) expression, Tᵢ is the difference of the ith tissue sample from the overall average and Gⱼ is the difference of the jth gene from the overall average, wherein Σᵢ₌₁ⁿTᵢ=0, Σⱼ₌₁^{g}Gⱼ=0, wherein εᵢ=(εᵢ₁, . . . ,ε_{ig})˜N(0,Σ), wherein

$$\sum = ( \begin{matrix}{σ}_{1} & … & {σ}_{g}\end{matrix} ) ( \begin{matrix}1 & ρ & … & ρ \\ ρ & 1 & … & ρ \\ ⋮ & & ⋰ & ⋮ \\ ρ & ρ & … & 1\end{matrix} ) \cdot ( \begin{matrix}{σ}_{1} \\ ⋮ \\ {σ}_{g}\end{matrix} ) ,$$

and wherein Σ is standard deviation; c) identifying a best gene within the set of genes having the lowest standard deviation, σⱼ, wherein the best gene represents the best housekeeper gene or expression control gene for the tissue.

Disclosed are methods of selecting a best housekeeper or control gene for a set of tissues, comprising a) obtaining expression data for a set of genes from a set of tissues; b) comparing the expression of each gene in each tissue using the equation: log yᵢ₍ₖ₎ⱼ=μ+Cₖ+Tᵢ₍ₖ₎+Gⱼ+(CG)ₖⱼ+εᵢ₍ₖ₎ⱼ, wherein (yᵢ₍ₖ₎ⱼ) represents an expression component of gene j in sample i of tissue type k to an overall mean (log-) expression, wherein μ denotes the overall mean (log-) expression, Cₖ is the difference of the kth tissue type from the overall average, Tᵢ₍ₖ₎ is the specific effect of the ith sample of tissue-type k, and Gⱼ is the difference of the jth gene from the overall average, (CG)ₖⱼ; wherein Σₖ₌₁^{m}Cₖ=0, Σᵢ₌₁ⁿ^{ₕ}Tᵢ₍ₖ₎=0, Σⱼ₌₁^{g}Gⱼ=0, Σⱼ₌₁^{g}(CG)ₖⱼ=Σₖ₌₁^{m}(CG)ₖⱼ=0, wherein εᵢ₍ₖ₎ⱼ˜N(0,σₖ²ζⱼ²) independent, ζ₁=1, c) identifying a best gene within the set of genes within the set of tissues having the lowest standard deviation, wherein the best gene represents the best housekeeper gene or expression control gene for the set of tissues.

Also disclosed are computerized implementing systems, as well as storage and retrieval systems, of biological information, comprising: a data entry means; a display means; a programmable central processing unit; and a data storage means having expression data for a gene electronically stored; wherein the stored sequences are used as input data for determining which sequence is the best housekeeper gene or expression control gene for a specific tissue type.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular expression control gene is disclosed and discussed and a number of modifications that can be made to a number of molecules including the expression control gene are discussed, specifically contemplated is each and every combination and permutation of expression control gene and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

3. Nucleic Acids

There are a variety of molecules disclosed herein that are nucleic acid based, including for example the nucleic acids that encode, for example, (MRPL19 (SEQ ID NO:1), PSMC4 (SEQ ID NO:2), SF3A1 (SEQ ID NO:3), PUM1 (SEQ ID NO:4), as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science, 1991, 254, 1497-1500).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the (MRPL19 (SEQ ID NO:1), PSMC4 (SEQ ID NO:2), SF3A1 (SEQ ID NO:3), PUM1 (SEQ ID NO:4) genes as well as the others disclosed herein and others are herein incorporated by reference in their entireties as well as for individual subsequences contained therein.

One particular sequence set forth in SEQ ID NO:1 is used herein, as an example, to exemplify the disclosed compositions and methods. It is understood that the description related to this sequence is applicable to any sequence related to SEQ ID NO:1 or the other genes disclosed herein, such as those in (MRPL19 (SEQ ID NO:1), PSMC4 (SEQ ID NO:2), SF3A1 (SEQ ID NO:3), PUM1 (SEQ ID NO:4), unless specifically indicated otherwise. Those of skill in the art understand how to resolve sequence discrepancies and differences and to adjust the compositions and methods relating to a particular sequence to other related sequences (i.e. sequences of (MRPL19 (SEQ ID NO:1), PSMC4 (SEQ ID NO:2), SF3A1 (SEQ ID NO:3), PUM1 (SEQ ID NO:4)). Primers and/or probes can be designed for any (MRPL19 (SEQ ID NO:1), PSMC4 (SEQ ID NO:2), SF3A1 (SEQ ID NO:3), PUM1 (SEQ ID NO:4) or other gene sequence given the information disclosed herein and known in the art.

4. Kits

Disclosed are kits comprising nucleic acids which can be used in the methods disclosed herein and, for example, buffers, salts, and other components to be used in the methods disclosed herein. Disclosed are kits for detecting the expression product of housekeeper genes and expressing control genes comprising nucleic acids which hybridize with the sequences in SEQ ID NOs:1-27. Also disclosed are kits, wherein the kits also comprises instructions.

5. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfa) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

6. Peptides

a) Protein Variants

As discussed herein there are numerous variants of the disclosed proteins that are known and herein contemplated. In addition, to the known functional strain variants there are derivatives of the disclosed proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | Ala; A |
| arginine | Arg; R |
| asparagine | Asn; N |
| aspartic acid | Asp; D |
| cysteine | Cys; C |
| glutamic acid | Glu; E |
| glutamine | Gln; Q |
| glycine | Gly; G |
| histidine | His; H |
| isoleucine | Ile; I |
| leucine | Leu; L |
| lysine | Lys; K |
| methionine | Met; M |
| phenylalanine | Phe; F |
| proline | Pro; P |
| serine | Ser; S |
| threonine | Thr; T |
| tyrosine | Tyr; Y |
| tryptophan | Trp; W |
| valine | Val; V |

TABLE 2
Amino Acid Substitutions
Original Residue Exemplary Conservative
Substitutions, others are known in the art.

Ala; Ser
Arg; Lys; Gln
Asn, Gln; His
Asp; Glu
Cys; Ser
Gln; Asn; Lys
Glu; Asp
Gly; Pro
His; Asn; Gln
Ile; Leu; Val
Leu; Ile; val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:9 sets forth a particular sequence of MRPL19. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:9 is set forth in SEQ ID NO:1. It is also understood that while no amino acid sequence indicates what particular DNA sequence encodes that protein within an organism, where particular variants of a disclosed protein are disclosed herein, the known nucleic acid sequence that encodes that protein in the particular specifies from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Tbba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

7. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6; 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

8. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

9. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

### C. \METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### D. METHODS OF USING THE COMPOSITIONS

1. Methods of Using the Compositions as Research Tools

The disclosed compositions can be used in a variety of ways as research tools. The compositions can be used for example as targets in combinatorial chemistry protocols or other screening protocols to isolate molecules that possess desired functional properties related to the disclosed genes.

The disclosed compositions can also be used diagnostic tools related to diseases, such as cancers, such as those listed herein.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis of for example, the genes disclosed herein. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### E. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used throughout, by a “subject” is meant an individual. Thus, the “subject” can include, for example, domesticated animals, such as cats, dogs, etc., livestock (e.g., cattle, horses, pigs, steep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout the description and claims of this specification, the word “comprise” and variations of the word, such as “comprising” and “comprises,” means “including but not limited to,” and is not intended to exclude, for example, other additives, components, integers or steps.

The term “cell” as used herein also refers to individual cells, cell lines, or cultures derived from such cells. A “culture” refers to a composition comprising isolated cells of the same or a different type.

The term “pro-drug” is intended to encompass compounds which, under physiologic conditions, are converted into therapeutically active agents. A common method for making a prodrug is to include selected moieties which are hydrolyzed under physiologic conditions to reveal the desired molecule. In other embodiments, the prodrug is converted by an enzymatic activity of the host animal.

The term “metabolite” refers to active derivatives produced upon introduction of a compound into a biological milieu, such as a patient.

When used with respect to pharmaceutical compositions, the term “stable” is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Statistical Modeling for Selecting Housekeeper Genes or Expression Control Genes

a) Results & Discussion

(1) One Tissue Type

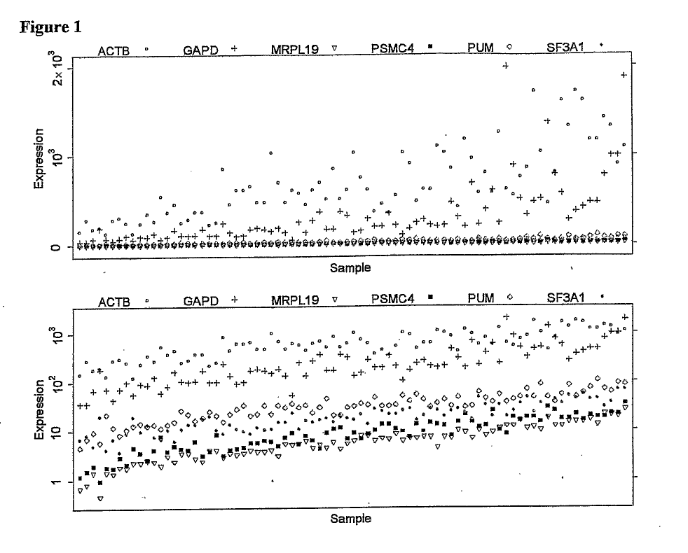

The genes MRPL19 (SEQ ID NO:1), PSMC4 (SEQ ID NO:2), SF3A1 (SEQ ID NO:3), PUM1 (SEQ ID NO:4), ACTB (SEQ ID NO:5) and GAPD (SEQ ID NO:6) were analyzed by real-time quantitative RT-PCR. Starting copy numbers for the 6 candidate housekeeping genes were measured across 80 primary breast tumor samples. Plots of the raw and log-scaled [All the logarithms are natural (base e) logarithms] expression levels are shown in FIG. 1. The samples were ordered according to the mean of the (log-) expression levels of all the genes. It is evident from the plot that for the raw data the variability of within-sample measurements increases with the mean expression, while the variability stays approximately the same for all the samples with the log-transformation. Additionally, the log-transformation allowed the modeling of fold-changes in an additive way.

To select the best housekeepers or control expressing genes for normalizing data across a single tissue type, 3 variations of a model (Model 1 a-c) were tested with real-time quantitative RT-PCR data generated from primary breast samples. A model (the assumptions are specified in detail below) of the expression yᵢⱼ of gene j in sample i by

Model 1a: log yᵢⱼ=μ+Tᵢ+Gⱼ+εᵢⱼ, where εᵢⱼ˜N(0,σⱼ²) was used.

Here μ denotes the overall mean (log-) expression, Tᵢ is the difference of the ith tissue sample from the overall average and Gⱼ is the difference of the jth gene from the overall average. The key feature of this model that makes it different from a traditional ANOVA model is that it allows heteroscedastic errors to account for different variability in the genes (Pinheiro J C BD: The Annals of Statistics 1978, 6:461-464). The variability around the gene-specific mean log-expression μ+Tᵢ+Gⱼ is quantified by the error standard deviation σⱼ. The Bayesian Information Criterion (BIC) was used to avoid overfitting the data (Schwarz G: Estimating the dimension of a model. The Annals of statistics 1978, 6:461-464).

Model 1a had the best BIC value and was selected from a range of competing models that included a method with equal error variances (Model 1b in Methods) and a more complex method with correlated errors (Model 1c in Methods).

Using Model 1a, standard deviations were determined to select the best control genes for breast cancer. Table 3 shows that MRPL19 has the smallest variability across the breast cancer samples and would be the best choice for a single housekeeper control or expression control gene.

TABLE 3
Standard deviation estimates of log expression using
Model 1a for selecting the single best housekeeper gene
or expression control gene for breast cancer.

| | Estimated standard | 95% confidence |
|---|---|---|
| Gene | deviation | interval |
| MRPL19 | 0.218 | (0.168, 0.284) |
| PUM1 | 0.265 | (0.215, 0.328) |
| PSMC4 | 0.288 | (0.235, 0.352) |
| SF3A1 | 0.393 | (0.327, 0.472) |
| ACTB | 0.448 | (0.376, 0.533) |
| GAPD | 0.519 | (0.439, 0.613) |

While some of the confidence intervals overlap, a direct comparison between the genes selected from the microarray (MRPL19, PSMC4, PUM1, SF3A1) to the classical housekeepers (GAPD and ACTB) shows significant difference (p=0.0014).

Since the biological function of many genes is still unknown, it is difficult to predict how different experimental conditions may affect the expression of putative housekeeper genes or expression control genes. Thus, a safer approach is to use an average expression of several genes that show small variance across conditions. Based on the selected model, the estimate of the variance of the log-average of the expression of several genes can be calculated (see Methods for details). Table 4 shows the standard deviations of the log-average of the best gene set for each possible set size (i.e., 1-6).

TABLE 4
Standard deviation estimates of log expression using
Model 1a for selecting the best housekeeper gene(s)
or expression control gene(s) for breast cancer.

| | | Standard |
|---|---|---|
| Set size | Gene set | deviation |
| 1 | MRPL19 | 0.2182 |
| 2 | PUM1, MRPL19 | 0.1718 |
| 3 | PSMC4, MRPL19, PUM1 | 0.1494 |
| 4 | PSMC4, MRPL19, PUM1, SP3A1 | 0.1490 |
| 5 | PSMC4, MRPL19, SF3A1, PUM1, ACTB | 0.1491 |
| 6 | PSMC4, MRPL19, SF3A1, PUM1, GAPD, ACTB | 0.1513 |

These standard deviation values are approximately equal to the coefficient of variation in the original scale. Based on the estimates, the 4-gene set of PSMC4, MRPL19, PUM1 and SF3A1 provides the lowest overall variability when choosing a combination of genes. However, this 4-gene set is barely different from the 3-gene combination of MRPL19, PUM1 and PSMC4, which in turn is far better than the best 2-gene combination. For economical reasons and the fact that SF3A1 had a relatively high individual variability compared to others in the set, a good choice for the normalizing set is the geometric mean of the expressions of MRPL19, PUM1 and PSMC4.

These findings illustrate the importance of performing an unbiased and genome-wide search for housekeepers rather than relying on traditional housekeeper genes or expression control genes. We used microarray data to select genes with low variability in expression across breast tumors and cell lines. Since the quantitative differences between the microarray and RT-PCR platforms are relative, genes with low variability in expression across tumors by microarray should also show low variability in expression by RT-PCR. Although the quantitative data from microarray tends to have an overall smaller dynamic range compared to RT-PCR, this is primarily due to loss of information from low expressed genes. The microarray dataset was filtered to remove low expressed genes with signals near background noise.

Using Vandesompele et al's M value method the result is very similar with only the positions of PUM1 and PSMC4 changing in stability rank. It should be noted that the M-value method does not order the two best genes (MRPL19 and PSMC4). Their best gene-set selection approach would suggest using the (log-scale) average of these two best genes as a control. A benefit to the disclosed methods is the ability to compare the variability of individual genes to that of an average of several genes.

(2) Multiple Tissue Types

Gene(s) with minimal variation in expression across different cell types serve as good “universal” housekeepers or expression control genes. A universal control may be a single gene or combination of genes. While the former typically displays both low variability within a given tissue type and consistent basal levels of expression across tissue types, the latter may be comprised of a gene set with individually different but complementary basal expression levels across tissue types.

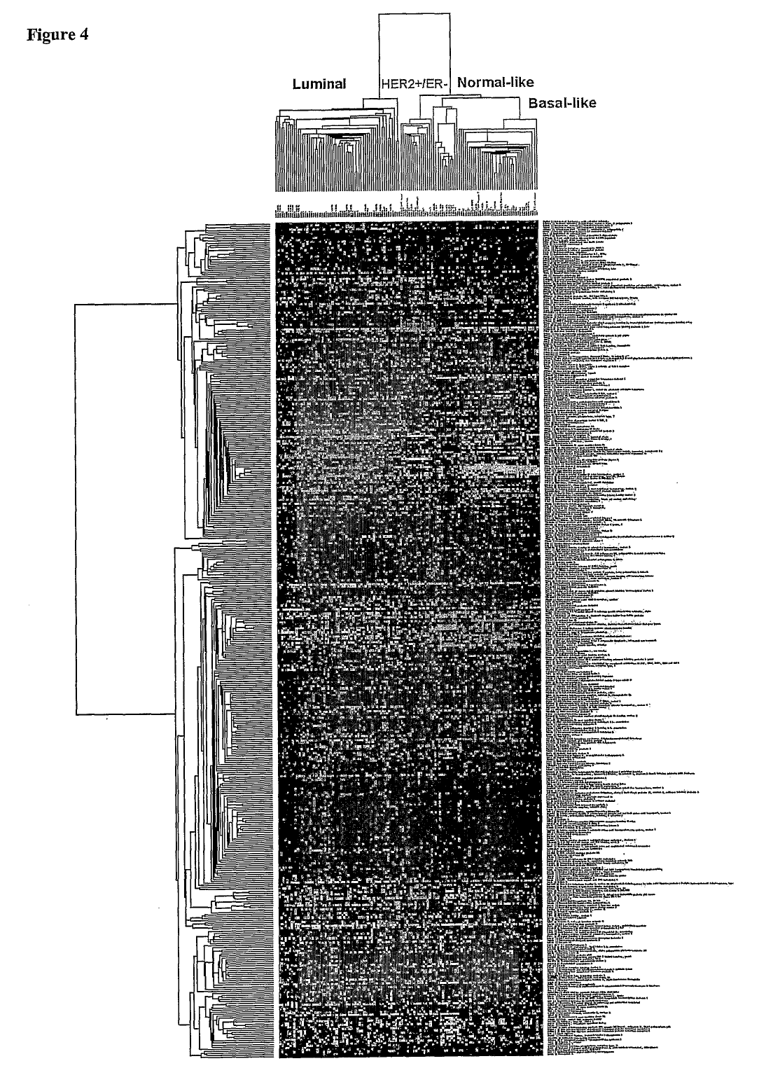

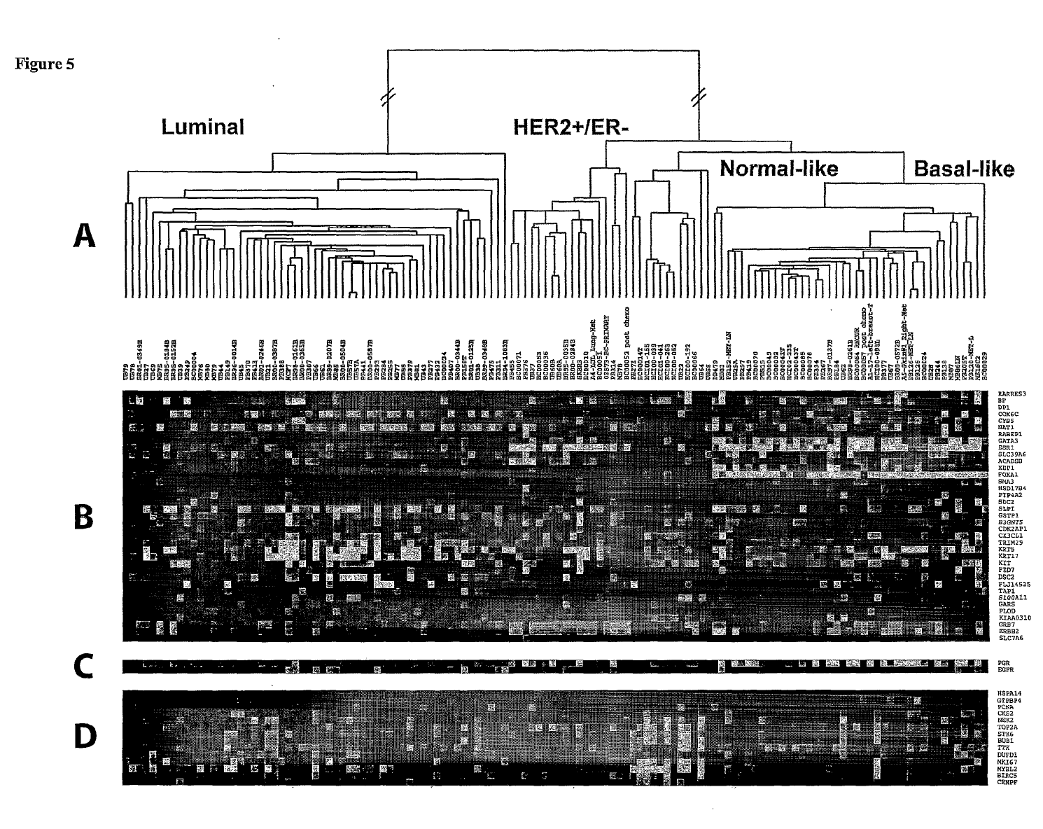

To test the models for selecting universal housekeepers, a published data set was used from Vandesompele et al (Vandesompele J, et al., Genome Biol 2002, 3:RESEARCH0034). They measured the expression level of 10 genes in neuroblastoma cell lines (NEU), cultured normal fibroblasts (FIB), normal leukocytes (LEU) and cells from normal bone marrow (BM). In addition, normal tissues from pooled organs (breast, brain, fetal brain, heart, kidney, uterus, lung, trachea and small intestine) were also profiled. A plot of these housekeepers or expression control genes across the different tissues is shown in FIG. 4. It is notable that a gene can have stable expression within a given tissue type but can change rank position compared to other housekeepers or expression control genes across tissues. For example, GAPD has relatively high expression in fibroblasts compared to other housekeepers or expression control genes but low expression in leukocytes. Thus, GAPD may be a good single housekeeper within certain tissue types but may not be an optimal universal housekeeper or expression control gene unless it is used within a complementary gene set.

To compare the performance of housekeepers or expression control genes within and between different tissues, the following was used as a model (The assumptions are specified in detail in the Methods section) the expression of gene j in the ith sample of tissue-type k by

Model 2: log yᵢ₍ₖ₎ⱼ=μ+Cₖ+Tᵢ₍ₖ₎+Gⱼ+(CG)ₖⱼ+εᵢ₍ₖ₎ⱼ, where εᵢ₍ₖ₎ⱼ˜N(0,σⱼ²ζₖ²).

Here μ denotes the overall mean (log-) expression, Cₖ is the difference of the kth tissue type from the overall average, Tᵢ₍ₖ₎ is the specific effect of the ith sample of tissue-type k, Gⱼ is the difference of the jth gene from the overall average and (CG)ₖⱼ is the tissue-type specific effect of gene j. Variability in calculation comes from two sources: the specific gene (σⱼ) and the tissue-type (ζₖ). The estimates of these parameters are given in Table 5.

TABLE 5
Components of the standard deviation estimates
of the log-expression of the Vandesompele data.
Standard deviation of genes (σⱼ)

| GAPD | UBC | HPRT1 | YWHAZ | SDHA | RPL13A | TBP | HMBS | ACTB | B2M |
|---|---|---|---|---|---|---|---|---|---|
| 0.211 | 0.226 | 0.227 | 0.232 | 0.255 | 0.339 | 0.339 | 0.431 | 0.460 | 0.562 |

Tissue-type specific multipliers (ζₖ)

| BM | FIB | NEU | LEU | POOL |
|---|---|---|---|---|
| 1.000 | 1.204 | 1.582 | 1.879 | 2.014 |

The single gene with the overall lowest variability within each tissue type is GAPD, followed closely by UBC, HPRT1 and YWHAZ. Here a rank of 1.5 was assigned to the unordered best pair and then average the ranks to obtain an overall ordering of the genes.

Mathematically, the risk of normalizing data to a housekeeper gene or expression control gene with variable overall expression level across different tissues can be represented as bias error. A housekeeper or expression control gene that has low bias for a particular tissue has an expression level that is near its mean expression across tissues. In the second model, the term (CG), represents this tissue-type specific bias. The measure of variability around an intended value when bias is present is called the mean squared error (MSE): MSE=Bias²+Variance. Thus, to find a set of genes for normalization across the various tissue types we use a minimax MSE criterion: minimizing the largest MSE of the combination. Table 6 provides a list for the best gene set of each size along with the minimax-MSE value.

TABLE 6
Minimax MSE optimal gene sets for each set-size.

| Max. number | | Maximal |
|---|---|---|
| of members | Gene set | MSE |
| 1 | RPL13A | 0.544 |
| 2 | HPRT1, UBC | 0.328 |
| 3 | HPRT1, RPL13A, UBC | 0.136 |
| 4 | HPRT1, RPL13A, UBC | 0.136 |
| 5 | HPRT1, RPL13A, UBC | 0.136 |
| 6 | ACTB, HPRT1, SDHA, TBP, UBC, YWHAZ | 0.131 |
| 7 | ACTB, HPRT1, RPL13A, SDHA, TBP, UBC, YWHAZ | 0.064 |
| 8 | ACTB, HPRT1, RPL13A, SDHA, TBP, UBC, YWHAZ | 0.064 |
| 9 | ACTB, HPRT1, RPL13A, SDHA, TBP, UBC, YWHAZ | 0.064 |
| 10 | ACTB, B2M, GAPD, HMBS, HPRT1, RPL13A, SDHA, TBP, UBC, YWHAZ | 0.049 |

Although GAPD has relatively low overall variability within each tissue type, its basal expression changes across tissue types making it a poor choice for a single universal control. The data shows that RPL13A is the best single universal housekeeper, but it is clear that no single gene is optimal for a universal housekeeper. Actually, choosing all the candidates provides the smallest MSE, which is not surprising since the set of all 10 genes is unbiased by definition. For routine application it is reasonable to limit the number of control genes, as the cost of saying additional genes needs to balance the extra precision obtained. With this in mind, it is instructive to note that the 3 member set of HPRT1, RPL13A, and UBC is an excellent choice because it maintains a priority ranking even when selection is open to including 4- or 5-element sets. The complete dataset of housekeepers were not tested across different tissue types so they could not be evaluated as universal controls. Nevertheless, it is likely that the results in breast tissue would hold-up across tissue types since the genes were initially selected from microarray data that included 17 different and diverse cell lines as well as primary breast tumors (Perou C M, Brown P O, Botstein D: Tumor classification using gene expression patterns from DNA microarrays. New Technologies for life sciences: A Trends Guide 2000:67-76).

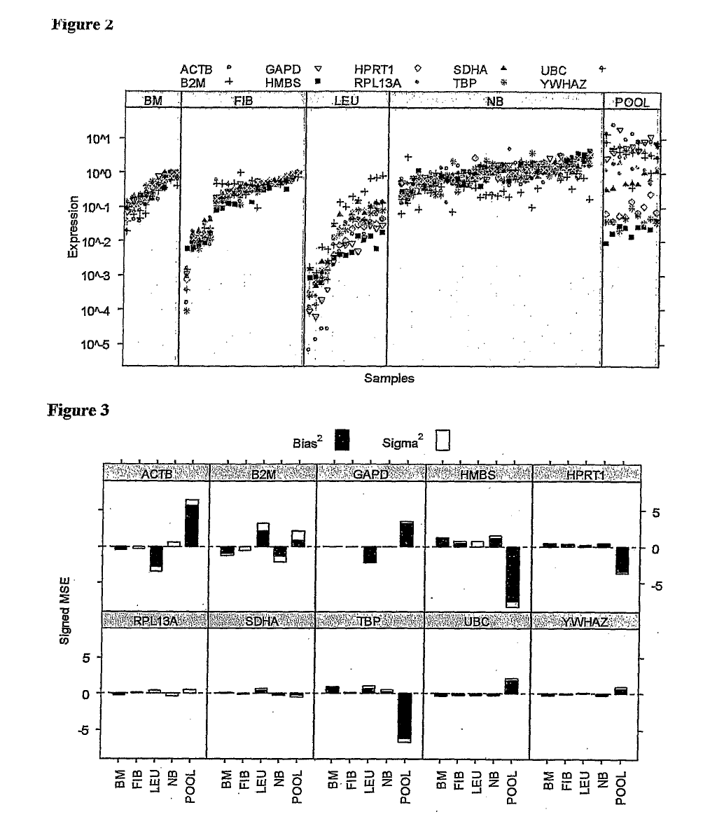

FIG. 3 shows the mean square error of each gene broken down into the squared-bias and variance components. The direction of each bar shows the sign of the bias. It is apparent that the large bias dominates the large values of MSE. The use of the (log-) average of several genes trends to reduce the variance, due to the effect bias-reduction where opposite biases cancel each other out. For example, both ACTB and TBP have a large bias in the pooled normal samples, but in opposing directions. The mean squared error of the (log-) average of ACTB and TBP in these samples is only 0.35, which is much lower than their individual MSE's above 6.

In summary, the performance of putative housekeepers or expression control genes were modeled to test goodness-of-fit in serving as normalization controls for relative quantitative analysis. A major advantage of a model approach is that the terms are placed within a solid statistical framework and are not ad hoc, which allows the algorithm to be generalized to a variety of different experimental conditions. The genes and algorithms that were selected for normalization have broad utility for diagnostics and research.

b) Material and Methods

(1) Pre-Selection of Assayed Genes from Microarray

Four candidate housekeepers (PSMC4, MRPL19, PUM1 and SF3A1) were selected from a microarray dataset containing 40 different breast tumors, 3 normal breast samples and 19 cell lines representing 17 different cell lines of diverse nature including lymphocytes, fibroblasts and epithelial cells (Perou C M, et al., Nature 2000, 406:747-752). All experiments were done using a common reference strategy where all experimental samples are compared to the same reference comprised of a pool of RNAs isolated from 11 diverse human cell lines (Perou C M, Brown P O, Botstein D: Tumor classification using gene expression patterns from DNA microarrays. New Technologies for life sciences: A Trends Guide 2000:67-76).

To select housekeepers or expression control genes, first the microarray data was “filtered” to select genes with Cy3 and Cy5 signal intensities greater than 500 units across at least 75% of the experiments. This requirement ensures that the gene is well expressed not only in the experimental samples, but also in the common reference sample. Next, the SAS/STAT Analysis Package Version 8 (SAS Institute Inc., Cary, N.C.) was used to identify a set of genes that showed a small range of expression across sample types and the least variance of the array-mean normalized log-ratios. For real-time RT-PCR, we selected 4 of the top 6 genes—“pumilio (Drosophila) homolog 1” (PUM1) (SEQ ID NO:4), “proteasome (prosome, macropain) 4” (PSMC4) (SEQ ID NO:2), “mitochondrial ribosomal protein L19” (MRPL19) (SEQ ID NO:1), and “splicing factor 3a” (SF3A) (SEQ ID NO:3). The 2 other low-variability genes identified in the data were “immediate early response 3” (SEQ ID NO:7) and “SRY (sex determining region Y)-box 2” (SEQ ID NO:8). These genes were not selected due to their potential for being differentially regulated under other conditions. However, GAPD (SEQ ID NO:6) and β-actin (SEQ ID NO:5), that are commonly used reference genes (Roux S, Pichaud et al., Endocrinology 1997, 138:1476-1482), were included in the set of candidate genes for comparison to the microarray selection.

(2) Samples and cDNA Preparation

Breast samples were acquired under informed consent and received at the Huntsman Cancer Institute (Salt Lake City, Utah) for gene expression analysis (University of Utah, IRB #8533). All specimens were expediently processed in pathology upon arrival from surgery. Samples were grossly dissected, procured by flash freezing in liquid nitrogen, and stored at −80° C. until RNA extraction. Approximately 50-100 mg cancer tissue was homogenized from each sample and total RNA was prepared using the RNeasy midi kit (Qiagen Inc., Valencia, Calif.). The integrity of RNA was determined using the RNA 6000 Nano LabChip kit (Agilent Technologies, Palo Alto, Calif.) and an Agilent 2100 Bioanalyzer. Two microliters of total RNA (50 ng/μL) were heated to 70° C. and 1 μL was loaded on the column. Degradation was evaluated using the signal of the 18S and 28S ribosomal peaks (Frank S G, Bernard, P. S.: Profiling Breast Cancer using Real-Time Quantitative PCR. In Rapid Cycle Real-Time PCR: Methods and Applications. Edited by S. Meuer W, C., Nakagawara, K. Heidelberg, Germany, Springer, 2003: pp 95-106).

First strand cDNA was synthesized from 1 μg total RNA using oligo-dT primers and Superscript III reverse transcriptase following manufacturer's instructions (Superscript III First-Strand Synthesis System, Invitrogen Life Technologies, Carlsbad, Calif.). Briefly, the reaction was held at 48° C. for 50 min, followed by a 15 min step at 70° C. The cDNA was washed on QIAquick PCR purification column (Qiagen) and eluted in 2×50 μl of Elution Buffer. The cDNA was then diluted in TE′ (10 mM Tris, 0.1 mM EDTA, pH 8.0), aliquoted and stored at −80° C. for further use.

(3) Real-Time Quantitative PCR

All PCR reactions were performed on the LightCycler. Each 20/L reaction included 1×PCR buffer with 3 mM MgCl₂ (Idaho Technology Inc; catalog #1770), 0.2 mM each of dATP, dCTP, and dGTP (Roche, Indianapolis, Ind., USA), 0.1 mM dTTP (Roche), 0.3 mM dUTP (Roche), 1U of Platinum taq (Invitrogen Life Technologies, Carlsbad, Calif.), 1/40000 SYBR Green I (Molecular probes, Eugene, Oreg.), approximately 5 ng cDNA, and 0.4 μM of each primer. The primers used for the RNA control genes are shown in Table 5.

PCR was done using the following protocol: initial denaturation 95° C. for 1 min 30 sec, then 50 cycles at 94° C. for 1 sec for denaturation, 60° C. for 5 sec (20° C./s transition) for annealing, 72° C. for 8 sec (2° C./sec transition) for extension. Fluorescence emission of SYBR Green I (channel 1-530 nm) was acquired each cycle after the extension step. A melting step was performed after PCR to determine product purity. For melting curve analysis, the reactions were rapidly (20° C./s) cooled from 95° C. to 60° C. and then slowly heated (0.1° C./s) back to 95° C. while continuously monitoring fluorescence.

(4) Relative Quantification

Copy number was determined using the crossing point (Cp) value, which is automatically calculated using the LightCycler 3.5 software (Roche Molecular Biochemicals). The Cp value is reported as a fractional cycle number that is determined from the 2^{nd} derivative maximum (point of maximum acceleration) on the PCR amplification curve (fluorescence versus cycle number) (Rasmussen R P: Quantification on the LightCycler. In Rapid Cycle Real-Time PCR: Methods and Applications. Edited by Wittwer C T, Meuer, S., Nakagawara, K. Heidelberg, Springer Verlag, 2001: pp 21-34). A relative starting copy number was determined for each housekeeper or expression control gene using a calibration curve done with the same batch of master mix. Efficiency (E) of PCR was calculated from a plot of Cp versus log ng cDNA (Rasmussen R P: Quantification on the LightCycler. In Rapid Cycle Real-Time PCR: Methods and Applications. Edited by Wittwer C T, Meuer, S., Nakagawara, K. Heidelberg, Springer Verlag, 2001: pp 21-34).
E=₁₀^{-1/slope}

(a) Modeling Expression Data

As the effects of interest are fold-changes, the log-transformed expression was modeled.
log yᵢⱼ=μ+Tᵢ+Gⱼ+εᵢⱼ,
Model 1a:

where Σᵢ₌₁ⁿTᵢ=0, Σⱼ₌₁^{g}Gⱼ=0, εᵢⱼ˜N(0,σⱼ²) independent

Here μ denotes the overall mean (log) expression, Tᵢ is the difference of the ith tissue sample from the overall average and Gⱼ is the difference of the jth gene from the overall average. The key feature of this model that makes it different from a traditional ANOVA model is that it allows heteroscedastic errors: the variability of the genes is different.

The model was fitted using the gls routine of the nlme library for R, however other commonly available software such as PROC MIXED from SAS could have been used.

Based on the model, the variability of the logarithm of the geometric mean \check{y}_{iS}=(Π_{jεS}yᵢⱼ)^{/|S|} of a gene-set S was estimated as

Vandesompele et al's M-value is the average of relative standard deviations of the log-expression levels. Under Model 1, the M-value of the gene is closely related to its variance (under Models 2 and 3 below, the similar relationships can be derived):

$$\begin{matrix}{V}_{jk} = SD ( {\{ log ( {y}_{ij} / {y}_{ik} ) \}}_{i = 1}^{n} ) = SD ( {\{ log ( {y}_{ij} ) - log ( {y}_{ik} ) \}}_{i = 1}^{n} ) \\ = \sqrt{{σ}_{j}^{2} + {σ}_{k}^{2}} \\ {M}_{j} = {\sum }_{k = 1 , … , {g}_{k \neq j}} {V}_{jk} / ( g - 1 ) = {σ}_{j}^{2} \frac{{\sum }_{k \neq j} \sqrt{1 + {σ}_{k}^{2} / {σ}_{j}^{2}}}{g - 1} \\ {σ}_{j}^{2} \sqrt{1 + 1 / {R}^{2}} \leq {M}_{j} \leq {σ}_{j}^{2} \sqrt{1 + {R}^{2}} , where R = {max}_{i , k} {σ}_{k} / {σ}_{i} \end{matrix}$$

The assumption of unequal variances was tested by fitting Model 1b that forces all the genes to have the same variability (this is the classical ANOVA model).
log yᵢⱼ=μ+Tᵢ+Gⱼ+εᵢⱼ,
Model 1b:

where Σᵢ₌₁ⁿTᵢ=0, Σⱼ₌₁^{g}Gⱼ=0, εᵢⱼ˜N(0,σ²) independent

Model 1c with a correlated error structure can be used to assess the assumption of (conditional) independence of the genes given the sample mean. If warranted, a more complicated correlation structure can be imposed.

$$log {y}_{ij} = μ + {T}_{i} + {G}_{j} + {ɛ}_{ij} , \\ where \\ {\sum }_{i = 1}^{n} {T}_{i} = 0 , {\sum }_{j = 1}^{g} {G}_{j} = 0 , {ɛ}_{i} = {( {ɛ}_{i 1} , … , {ɛ}_{ig} )}^{′} ∼ N ( 0 , \sum ) \\ and \\ \sum = ( {σ}_{1} … {σ}_{g} ) ( \begin{matrix}1 & ρ & … & ρ \\ ρ & 1 & … & ρ \\ ⋮ & & ⋰ & ⋮ \\ ρ & ρ & … & 1\end{matrix} ) \cdot ( \begin{matrix}{σ}_{1} \\ ⋮ \\ {σ}_{g}\end{matrix} ) \qquad Model 1 c$$

For the multiple tissue-type setup the notation and the model need to be extended. The expression level of gene j of in the ith sample of type k were denoted by yᵢ₍ₖ₎ⱼ, i=1, . . . ,nₖ, j=1, . . . ,g and k=1, . . . ,m. The best-fitting model for the data had the form
log yᵢ₍ₖ₎ⱼ=μ+Cₖ+Tᵢ₍ₖ₎+Gⱼ+(CG)ₖⱼ+εᵢ₍ₖ₎ⱼ,
Model 2: where Σₖ₌₁^{m}Cₖ=0, Σᵢ₌₁ⁿ^{ₖ}Tᵢ₍ₖ₎=0, Σⱼ₌₁^{g}Gⱼ=0, Σⱼ₌₁^{g}(CG)ₖⱼ=Σₖ₌₁^{m}(CG)ₖⱼ=0, εᵢ₍ₖ₎ⱼ˜N(0,σk²ζⱼ²) independent, ζ₁=1.

Thus the errors are independent and their variability is decomposed into a gene-specific and tissue-type specific multiplicative components. The last restriction ensures the uniqueness of the solution. Other considered models were three simpler ones: uniform error variance, equal error variance for the tissue-types, equal error variance for genes and two more complex: exchangeable correlation structure for the errors and unstructured error variance (each gene-tissue-type combination has a variance parameter). The Bayesian Information Criterion was used as a basis for model selection.

### 2. Example 2

### Biological Classification of Breast Cancer by Real-Time Quantitative RT-PCR: Comparisons to Microarray and Histopathology

a) Methods

Patient selection. An ethnically diverse cohort of patients were studied using samples collected from various locations throughout the United States. Tissues analyzed included 117 invasive breast cancers, 1 fibroadenoma, 5 “normal” samples (from reduction mammoplasty), and 3 cells lines. Patients were heterogeneously treated in accordance with the standard of care dictated by their disease stage, ER and HER2 status. Patients were censored for recurrence and/or death for up to 118 months (median 21.5 months). Clinical data presented in supplementary Table 7.

SAMPLES CLINICAL DATA

| | | | ER (1 = positive; | | | | | | |
|---|---|---|---|---|---|---|---|---|---|
| | | | 0 = negative): if | | | Size (1 = <= 2 cm; | | | |
| | | | fmol = 10 = + | | | 2 => cm to <=5 cm; | | RFS event | |
| | | | (used fmol for rosetta and | | | 3 => 5 cm; 4 = any | | (0 = no relapse, | |
| | | | singapore) and norway | | | size with direct | | 1 = relapsed | |
| | | | as detailed in PNAS | | | extension to chest | | or died of | RFS |
| Sample Name | Age | Race | 2003 Table | HER2 | PGR | wall or skin) | Grade | disease) | months |
| 02573-BC-PRIMARY | 41 | AA | 1 | | | 3 | 3 | 1 | 10 |
| A1-17-left-breast-T | 64 | C | 0 | 0 | | 4 | 3 | 1 | 2 |
| A4-LUL_Lung-Met | 44 | C | 1 | | | 4 | 3 | 1 | 22 |
| A5-Skin#1_Right-Met | 65 | AA | 0 | | | 4 | 3 | 1 | 26 |
| BC00010 | 47 | C | 0 | | | 3 | 2 | 1 | 16 |
| BC00014T | 69 | AA | 0 | | | 4 | 3 | 1 | 18 |
| BC00024 | 68 | AA | 0 | | | 3 | 3 | 1 | 3 |
| BC00029 | 44 | C | 0 | | 0 | | 3 | 0 | 62 |
| BC00034 | 68 | AA | 0 | | | 1 | 2 | 0 | 81 |
| BC00036 | 55 | AA | 0 | | | 2 | 2 | 0 | 10 |
| BC0004 | 67 | C | 0 | | | 1 | 2 | 0 | 118 |
| BC00041T | 46 | AA | 0 | 0 | | 2 | 3 | 1 | 13 |
| BC00043T | 43 | C | 0 | | | 2 | 3 | 0 | 76 |
| BC00049 | 43 | C | 0 | | | 2 | 3 | 1 | 48 |
| BC00051 | 51 | C | 0 | | | 2 | 2 | 0 | 68 |
| BC00052 post chemo | 47 | AA | 0 | 2 | | 2 | 3 | 1 | 14 |
| BC00053 | 71 | AA | 0 | | | 2 | 3 | 1 | 27 |
| BC00057 post chemo | 51 | AA | 0 | 3 | | 4 | 3 | 1 | 8 |
| BC00064 RECUR | 44 | C | 0 | 2 | | 1 | 3 | 1 | 10 |
| BC00066 | 43 | AA | 0 | 3 | | 3 | 3 | 1 | 18 |
| BC00070 | 38 | AA | 0 | 0 | | 2 | 2 | 1 | 22 |
| BC00071 | 33 | C | 0 | | | 2 | 2 | 1 | 16 |
| BC00078 | 68 | AA | 0 | 0 | | 3 | 3 | 0 | 12 |
| BC00082 | 84 | AA | 0 | 0 | | 2 | 3 | 0 | 27 |
| BC00085 | 24 | AA | 0 | 1 | | 1 | 2 | 0 | 19 |
| BR00-0344B | 65 | AA | 1 | 0 | 0 | 2 | 3 | 1 | 7 |
| BR00-0365B | 43 | AA | 1 | 2 | 1 | 4 | 3 | 0 | 22 |
| BR00-0387B | 57 | AA | 1 | 0 | 1 | 4 | 2 | 1 | 6 |
| BR00-0504B | 88 | C | 1 | 0 | 1 | 2 | 1 | 0 | 39 |
| BR00-0572B | 45 | AA | 0 | 3 | 0 | 3 | 3 | 1 | 11 |
| BR00-0587B | 68 | C | 1 | 2 | 1 | 2 | 3 | 0 | 37 |
| BR00-284B | 63 | C | 0 | 3 | 0 | 3 | 3 | 0 | 43 |
| BR01-0125B | 40 | C | 1 | 0 | 1 | 3 | 2 | 0 | 33 |
| BR01-0246B | 36 | Other | 1 | 0 | 1 | 2 | 2 | 0 | 31 |
| BR01-0349B | 37 | C | 1 | 3 | 0 | 3 | 3 | 1 | 3 |
| BR94-1083B | 48 | C | 0 | 3 | 1 | 1 | 3 | 1 | 23 |
| BR95-0035B | 74 | C | 0 | 3 | 0 | 2 | 3 | 0 | 106 |
| BR95-0152B | 72 | C | 1 | 3 | 0 | 4 | 3 | 1 | 26 |
| BR95-0184B | 74 | C | 1 | 3 | 0 | 2 | 3 | 0 | 96 |
| BR96-0014B | 47 | AA | 1 | 3 | 1 | 4 | 1 | 0 | 96 |
| BR97-0137B | 55 | Other | 0 | | 0 | 3 | 3 | 1 | 20 |
| BR98-0161B | 57 | AA | 0 | 3 | 0 | 2 | 2 | 1 | 36 |
| BR98-0261B | 44 | C | 0 | 3 | 0 | 2 | 3 | 0 | 65 |
| BR99-0207B | 84 | C | 1 | 0 | 0 | 2 | 2 | 0 | 57 |
| BR99-0348B | 85 | AA | 1 | 2 | 1 | 2 | 2 | 0 | 32 |
| HCl00-039 | | | | | | | | | |
| HCl00-052 | | | | | | | | | |
| HCl00-098L | | | | | | | | | |
| HCl00-192 | | | | | | | | | |
| HCl00-263 | | | | | | | | | |
| HCl01-041 | | | | | | | | | |
| HCl01-155 | | | | | | | | | |
| HCl02-235 | 57 | C | 0 | 0 | 0 | 2 | 3 | | |
| HCl02-264 | 50 | C | 1 | 1 | 1 | 3 | 0 | 1 | 0 |
| MB75 | 53 | | 1 | 3 | 1 | 2 | 3 | 0 | 20 |
| MB76 | 57 | | 1 | 0 | 1 | 2 | 2 | 0 | 22 |
| MB77 | 63 | | 1 | 0 | 1 | 2 | 3 | 0 | 22 |
| MB78 | 56 | | 0 | 2 | 0 | 2 | 3 | | |
| MB79 | 63 | | 1 | 3 | 1 | 2 | 3 | 0 | 18 |
| MB80 | 58 | | 1 | 0 | 1 | 2 | 3 | | |
| MB81 | 84 | | 1 | 0 | 1 | 2 | 2 | 1 | 13 |
| MB83 | 31 | | 0 | 0 | 0 | 2 | 3 | | |
| MB85 | 77 | | 1 | 0 | 1 | 2 | 3 | | |
| MB86(LN) | 72 | | 0 | 0 | 0 | | 3 | 1 | 15 |
| MB87 | 73 | | 0 | 0 | 0 | | 3 | | |
| PB120-MET-L | 61 | AA | 0 | | | 2 | 3 | 1 | 1 |
| PB126 | 29 | AA | 0 | 0 | 0 | 4 | 3 | 1 | 1 |
| PB126-MET-LN | | AA | 0 | 0 | 0 | 4 | 3 | 1 | 1 |
| PB138 | 58 | C | 0 | 0 | 0 | 2 | 2 | 0 | 30 |
| PB149 | 41 | C | 1 | 2 | 1 | 2 | 1 | 0 | 34 |
| PB152-MET-LN | | C | 0 | 1 | 0 | | | | |
| PB158T | 86 | AA | 1 | | | 3 | 3 | 0 | 30 |
| PB184 | 50 | C | 1 | 3 | 0 | 1 | 3 | 0 | 29 |
| PB205T | 39 | C | 0 | 1 | 0 | 4 | 2 | 0 | 5 |
| PB244 | 38 | AA | 0 | 3 | 0 | 1 | 3 | 0 | 24 |
| PB249 | 36 | C | 1 | 3 | 1 | 1 | 3 | 0 | 8 |
| PB255 | 56 | C | 1 | 2 | 1 | 2 | 3 | 0 | 4 |
| PB267 | 44 | AA | 0 | 2 | 0 | 2 | 3 | 0 | 20 |
| PB271 | 45 | AA | 1 | 3 | 1 | 2 | 3 | 0 | 14 |
| PB277 | 46 | C | 1 | 2 | 1 | 2 | 3 | 0 | 12 |
| PB284 | 34 | C | 1 | 2 | 1 | 1 | 1 | | |
| PB293 | 56 | C | 1 | 2 | 1 | 2 | 2 | 0 | 11 |
| PB297 | 55 | AA | 0 | 1 | 0 | 2 | 3 | 0 | 18 |
| PB307 | 35 | | 1 | 1 | 1 | 3 | 3 | 0 | 9 |
| PB311 | 48 | C | 1 | 0 | 1 | 3 | 3 | 0 | 14 |
| PB314 | 51 | C | 0 | 3 | 0 | 3 | 3 | 0 | 21 |
| PB334 | 50 | AA | 0 | 0 | 0 | 1 | 3 | 0 | 19 |
| PB370 | 67 | C | 1 | 0 | 1 | 2 | 3 | 0 | 20 |
| PB376 | 50 | AA | 0 | 1 | 0 | 2 | 3 | 0 | 15 |
| PB377 | 77 | C | 1 | 1 | 0 | 2 | 3 | 0 | 18 |
| PB379 | 55 | Other | 1 | 1 | 1 | 2 | 3 | 0 | 17 |
| PB388 | 80 | C | 1 | 1 | 1 | 2 | 3 | 0 | 16 |
| PB407 | 56 | C | 1 | 0 | 1 | 3 | 3 | | |
| PB413 | 63 | AA | 1 | 0 | 1 | 2 | 2 | 0 | 9 |
| PB419 | 49 | C | 0 | 0 | 0 | 2 | 3 | 0 | 10 |
| PB432 | 79 | | 1 | 1 | 1 | 2 | 3 | | |
| PB441 | 83 | C | 1 | 0 | 1 | 1 | 2 | 0 | 9 |
| PB455 | 52 | AA | 0 | 3 | 0 | 3 | 2 | 0 | 9 |
| PB475 | 50 | C | 1 | 0 | 1 | 2 | 2 | 0 | 2 |
| PB479 | 52 | Asian | 1 | 0 | 1 | 2 | 3 | | |
| PB515 | | AA | 0 | 0 | 0 | 3 | 3 | | |
| UB21 | 77 | | 1 | 0 | 1 | 1 | 1 | 0 | 30 |
| UB22 | | | | | | | | 0 | 25 |
| UB27 | 91 | C | 1 | 2 | 1 | 3 | 2 | 0 | 29 |
| UB28 | 46 | C | 0 | 0 | 0 | 1 | 3 | 0 | 30 |
| UB29A | 59 | C | 0 | 0 | 0 | 2 | 3 | 0 | 25 |
| UB37 | 42 | C | 0 | 2 | 1 | 1 | 3 | 0 | 25 |
| UB38 | 50 | C | 1 | 0 | 1 | 1 | 1 | 0 | 20 |
| UB39 | 48 | C | 1 | 0 | 0 | 1 | 2 | 0 | 25 |
| UB43 | 48 | C | 1 | 1 | 1 | 1 | 3 | 0 | 19 |
| UB44 | 50 | C | 1 | 0 | 1 | 2 | 2 | 0 | 24 |
| UB45 | 46 | C | 1 | 1 | 1 | 2 | 2 | 0 | 21 |
| UB55 | 58 | C | 1 | 2 | 1 | 1 | 1 | 0 | 22 |
| UB57 | 60 | C | 1 | 0 | 1 | 1 | 2 | 0 | 17 |
| UB58 | 58 | C | 1 | 1 | 1 | 1 | 1 | 0 | 19 |
| UB60 | 72 | C | 0 | 3 | 0 | 2 | 3 | 0 | 20 |
| UB61 | 51 | C | 1 | 3 | 0 | 2 | 2 | 0 | 19 |
| UB62 | 28 | C | | | | 1 | 1 | 0 | 9 |
| UB64 | 87 | C | 1 | 3 | 1 | 2 | 2 | 0 | 7 |
| UB66 | 88 | Other | 1 | 0 | 1 | 2 | 1 | 0 | 9 |
| UB67 | 80 | C | 0 | 0 | 0 | 1 | 3 | 0 | 16 |
| UB69 | 40 | C | 1 | 0 | 0 | 1 | 1 | 0 | 13 |
| UB78 | 41 | hisp | 1 | 0 | 0 | 4 | 2 | 1 | 0 |
| UB79 | 46 | | 1 | 1 | 0 | 2 | 2 | 0 | 2 |

| | | | Overall | | |
|---|---|---|---|---|---|
| | | | Survival | | |
| | | number of | Event | | |
| | number of | nodes | (0 = alive, | Overall | |
| | nodes | positive | 1 = DOD or | suvival | |
| Sample Name | examined | for tumor | DOC) | months | Important Comments |
| 02573-BC-PRIMARY | 25 | 14 | 0 | 22 | primary for a patient with an associated |
| | | | | | brain [symbol] |
| A1-17-left-breast-T | | | 1 | 2 | Autopsy Patient Sample |
| A4-LUL_Lung-Met | | | 1 | 22 | Autopsy Patient Sample |
| A5-Skin#1_Right-Met | 14 | 3 | 1 | 26 | Autopsy Patient Sample |
| BC00010 | 21 | 19 | 1 | 16 | |
| BC00014T | 40 | 36 | 1 | 23 | |
| BC00024 | 116 | 14 | 1 | 3 | pt was diagnosed with MM at same time |
| | | | | | as br [symbol] |
| BC00029 | 7 | 3 | 0 | 62 | lymph node met sample? - no primary |
| | | | | | tumor [symbol] |
| BC00034 | | | 0 | 81 | |
| BC00036 | 23 | 1 | 0 | 10 | |
| BC0004 | 20 | 0 | 0 | 118 | |
| BC00041T | 19 | 0 | 1 | 29 | |
| BC00043T | 24 | 0 | 0 | 76 | |
| BC00049 | 13 | 1 | 0 | 72 | her2 was 1+ on recurrent tumor, not done |
| | | | | | on i [symbol] |
| BC00051 | 12 | 12 | 0 | 68 | |
| BC00052 post chemo | 13 | 9 | 1 | 18 | pt had LABC, had neoadj chemo, this |
| | | | | | specim [symbol] |
| BC00053 | 21 | 7 | 1 | 28 | |
| BC00057 post chemo | 9 | 9 | 1 | 12 | pt had IBC, had neoadj chemo, this specimen |
| BC00064 RECUR | | | 1 | 47 | pt had local recurrence (this is the sample |
| | | | | | we [symbol] |
| BC00066 | 38 | 4 | 1 | 18 | |
| BC00070 | | | 1 | 25 | contralateral breast cancer dx Nov. 15, 2000, |
| | | | | | dx wi [symbol] |
| BC00071 | 20 | 4 | 1 | 47 | |
| BC00078 | 16 | 12 | 1 | 12 | cirrhosis was cause of death |
| BC00082 | 3 | 0 | 1 | 27 | pt admitted with CHF/NQWMI, prob died of |
| | | | | | ar [symbol] |
| BC00085 | | | 0 | 19 | extensive DCIS w/multlple small foci of |
| | | | | | invasi [symbol] |
| BR00-0344B | 15 | 2 | 1 | 30 | |
| BR00-0365B | 8 | 6 | 0 | 22 | |
| BR00-0387B | 17 | 10 | 0 | 51 | |
| BR00-0504B | 15 | 1 | 0 | 39 | |
| BR00-0572B | 31 | 7 | 0 | 42 | |
| BR00-0587B | 14 | 0 | 0 | 37 | |
| BR00-284B | 6 | 0 | 0 | 43 | |
| BR01-0125B | 17 | 1 | 0 | 33 | |
| BR01-0246B | 16 | 9 | 0 | 31 | |
| BR01-0349B | 24 | 22 | 1 | 24 | |
| BR94-1083B | 19 | 1 | 1 | 47 | |
| BR95-0035B | 13 | 1 | 0 | 106 | |
| BR95-0152B | 15 | 0 | 0 | 101 | |
| BR95-0184B | 20 | 1 | 0 | 96 | |
| BR96-0014B | | | 0 | 96 | |
| BR97-0137B | 21 | 1 | 1 | 21 | died of Unconfirmed met ca (symptoms of |
| | | | | | me [symbol] |
| BR98-0161B | 24 | 0 | 1 | 60 | |
| BR98-0261B | 14 | 0 | 0 | 65 | |
| BR99-0207B | 5 | 1 | 0 | 57 | |
| BR99-0348B | 33 | 0 | 0 | 32 | died of other causes (dehydration secondary |
| | | | | | to [symbol] |
| HCl00-039 | | | | | |
| HCl00-052 | | | | | |
| HCl00-098L | | | | | |
| HCl00-192 | | | | | |
| HCl00-263 | | | | | |
| HCl01-041 | | | | | |
| HCl01-155 | | | | | |
| HCl02-235 | 12 | 0 | | | |
| HCl02-264 | 20 | 0 | | | ER positive tumor (5 cml) but no positive node |
| MB75 | 15 | 0 | 0 | 20 | |
| MB76 | 11 | 0 | 0 | 22 | |
| MB77 | 17 | 0 | 0 | 22 | Had right breast radical mastectomy in 1979l, |
| | | | | | [symbol] |
| MB78 | 5 | 4 | | | |
| MB79 | 7 | 0 | 0 | 16 | |
| MB80 | 0 | 0 | | | |
| MB81 | 1 | 1 | 0 | 16 | Several recurences (cutaneous, gastric) |
| MB83 | 17 | 0 | | | |
| MB85 | 11 | 2 | | | |
| MB86(LN) | 17 | 7 | 0 | 41 | Lymph node metastasis - Several recurrences |
| MB87 | 1 | 1 | | | metastasis in small intestine |
| PB120-MET-L | 15 | 14 | 1 | 13 | lymph node metastasis sample this patient |
| | | | | | wa [symbol] |
| PB126 | 7 | 7 | 1 | 16 | This patient was never disease-free and died |
| PB126-MET-LN | 7 | 7 | 1 | 16 | |
| PB138 | 0 | 0 | 0 | 30 | |
| PB149 | 10 | 0 | 0 | 34 | |
| PB152-MET-LN | | | | | ER, Her2 and PGR are for PB152 but maybe |
| PB158T | 0 | 0 | 0 | 30 | |
| PB184 | 2 | 0 | 0 | 29 | |
| PB205T | 7 | 1 | 0 | 5 | |
| PB244 | 12 | 0 | 0 | 24 | |
| PB249 | 3 | 3 | 0 | 8 | |
| PB255 | 14 | 1 | 0 | 4 | |
| PB267 | 32 | 1 | 0 | 20 | |
| PB271 | 12 | 3 | 0 | 14 | |
| PB277 | 18 | 9 | 0 | 12 | |
| PB284 | 0 | 0 | | | |
| PB293 | 12 | 0 | 0 | 11 | |
| PB297 | 0 | 0 | 0 | 18 | |
| PB307 | 15 | 0 | 0 | 9 | |
| PB311 | 12 | 2 | 0 | 14 | |
| PB314 | 13 | 8 | 0 | 21 | |
| PB334 | 0 | 0 | 0 | 19 | |
| PB370 | 11 | 2 | 0 | 20 | |
| PB376 | 3 | 0 | 0 | 15 | |
| PB377 | 8 | 0 | 0 | 18 | there are 2 different tumors within the same |
| | | | | | b [symbol] |
| PB379 | 12 | 4 | 0 | 17 | |
| PB38B | 5 | 0 | 0 | 18 | |
| PB407 | 11 | 6 | | | |
| PB413 | 9 | 3 | 0 | 9 | |
| PB419 | 1 | 0 | 0 | 10 | |
| PB432 | 21 | 4 | | | |
| PB441 | 0 | 0 | 0 | 9 | bilateral breast cancer and renal carcinoma |
| PB455 | 8 | 3 | 0 | 9 | |
| PB475 | 5 | 0 | 0 | 2 | |
| PB479 | 19 | 1 | | | |
| PB515 | 14 | 2 | | | IDC and DCIS |
| UB21 | 1 | 0 | 0 | 30 | |
| UB22 | | | | 25 | no malig (fibroadenoma) |
| UB27 | 14 | 2 | 0 | 29 | |
| UB28 | 20 | 0 | 0 | 30 | |
| UB29A | 19 | 0 | 0 | 25 | |
| UB37 | 14 | 3 | 0 | 25 | |
| UB3B | 13 | 0 | 0 | 20 | |
| UB39 | 10 | 0 | 0 | 25 | |
| UB43 | 14 | 14 | 0 | 19 | |
| UB44 | 3 | 1 | 0 | 24 | Had the other breast removed (contained |
| | | | | | mic [symbol] |
| UB45 | 5 | 1 | 0 | 21 | Had a second small tumor (5 mm - grade 1 - |
| | | | | | H [symbol] |
| UB55 | 4 | 0 | 0 | 22 | |
| UB57 | 2 | 0 | 0 | 17 | |
| UB58 | 4 | 1 | 0 | 19 | Graded 1 on the tissue we received (then |
| | | | | | got [symbol] |
| UB60 | 13 | 10 | 0 | 20 | |
| UB61 | 15 | 9 | 0 | 19 | |
| UB62 | 23 | 1 | 0 | 9 | No evidence of malignancy (we had IHC value |
| | | | | | [symbol] |
| UB64 | 15 | 0 | 0 | 7 | No follow-up visit (person out of state) |
| UB66 | 18 | 0 | 0 | 9 | (From Price, utah-chest X-ray visit used as |
| | | | | | La [symbol] |
| UB67 | 15 | 1 | 0 | 16 | |
| UB69 | 3 | 0 | 0 | 13 | (Can't find IHC data in the database to |
| | | | | | confir [symbol] |
| UB78 | 20 | 20 | 0 | 14 | has bone metastasis, in abdomen and pelvis - |
| UB79 | 9 | 2 | 0 | 2 | Macro-metastasis in the lymphanodes - Not |
| | | | | | fa [symbol] |

Sample preparation and first strand synthesis for qRT-PCR. Nucleic acids were extracted from fresh frozen tissue using RNeasy Midi Kit (Qiagen Inc., Valencia, Calif.). The quality of RNA was assessed using the Agilent 2100 Bioanalyzer with the RNA 6000 Nano LabChip Kit (Agilent Technologies, Palo Alto, Calif.). All samples used had discernable 18S and 28S ribosomal peaks. First strand cDNA was synthesized from approximately 1.5 mg total RNA using 500 ng Oligo(dT) 12-18 and Superscript III reverse transcriptase (1st Strand Kit, Invitrogen, Carlsbad, Calif.). The reaction was held at 42° C. for 50 min followed by a 15-min step at 70° C. The cDNA was washed on a QIAquick PCR purification column and stored at −80° C. in TE′ (25 mM Tris, 1 mM EDTA) at a concentration of 5 ng/ul (concentration estimated from the starting RNA concentration used in the reverse transcription).

Primer design. Genbank sequences were downloaded from Evidence viewer (NCBI website) into the Lightcycler Probe Design Software (Roche Applied Science, Indianapolis, Ind.). All primer sets were designed to have a Tm >>60° C., GC content >>50% and to generate a PCR amplicon <200 bps. Finally, BLAT and BLAST searches were performed on primer pair sequences using the UCSC Genome Bioinformatics (http://genome.ucsc.edu/) and NCBI (http://www.ncbi.nlm.nih.gov/BLAST/) to check for uniqueness. Primer sets and identifiers are provided in supplementary Table 8.

TABLE 8

Primer Sets and Gene ID

Gene
ID

| | | | | |
|---|---|---|---|---|
| Gene symbol | Gene name | (NCBI) | Forward primer | Reverse primer |

| | | | | |
|---|---|---|---|---|
| Intrinsic | | | | |
| gene list | | | | |
| ACADSB | Acyl-Coenzyme A dehydrogenase, | 36 | CTA ACA TAC AAT GCT | CAA TCT TTG CAT CTC |
| | short/branched chain | | GCT AGG C | GGA AGT |
| B3GNT5 | UDP-GlcNAc:betaGal beta-1,3-N- | 84002 | AGA ACT AGG TGG TGT | GAT TTT CCC TAA CAG |
| | acetylglucosaminyltransferase 5 | | CTA C | GTG C |
| BF | B factor, properdin | 629 | CAT GTG TTC AAA GTC | TGC TTG TGG TAA TCG |
| | | | AAG GAT A | GT |
| C5ORF18 | chromosome 5 open reading frame 18 | 7905 | GTG TTC GGT TAT GGA | GGT ATC ATC TTC TTT |
| (=DP1) | | | GC | GTT GGG A |
| CDK2AP1 | CDK2-associated protein 1 | 8099 | CGC AGG GAG CAA GAG | CTT CAA AAC CAA CAA |
| | | | T | GGC AG |
| COX6C | cytochrome c oxidase subunit Vlc | 1345 | AGC TTT GTA TAA GTT | CCA GCC TTC CTC ATC |
| | | | TCG TGT | TC |
| CX3CL1 | Chemokine (C-X3-C motif) ligand 1 | 6376 | ATG ACA TCA AAG ATA | GAC CCA TTG CTC CTT |
| | | | CCT GTA G | CG |
| CYB5 | cytochrome b-5 | 1528 | GCA CCA CAA GGT GTA | GCC CGA CAT CCT CAA |
| | | | CG | AG |
| DSC2 (ESTs) | Desmocollin2 | 1824 | GAA TGT GGA GAC TGA | CAA ATG GAG GAT CAT |
| | | | AAG CAA | TCT GAT AGG |
| EGFR | Epidermal growth factor receptor | 1956 | AGG ACA GCA TAG ACG | AGG ATT CTG CAC AGA |
| | (erythroblastic leukemia viral | | ACA C | GCC A |
| | (v-erb-b) oncogene homolog, avian) | | | |
| ERBB2 | V-erb-b2 erythroblastic leukemia | 2064 | TCC TGT GTG GAC CTG | TGC CGT CGC TTG ATG |
| | viral oncogene homolog 2, neuro/ | | GAT | AG |
| | glioblastoma derived oncogene | | | |
| | homolog (avian) | | | |
| ESR1 | Estrogen receptor 1 | 2099 | CATGATCAGGTCCACCTTCT | AGCAGCATGTCGAAGATCTC |
| FLJ14525 | Hypothetical protein FLJ14525 | 84886 | CCC TTT CTC CTG GGA | GCT TTG GAC AGT GGT |
| | | | AAC | CT |
| FOXA1 | Forkhead box A1 | 3169 | GTTAGGAACTGTGAAGATGG | GCCGCTCGTAGTCATG |
| FZD7 | Frizzled homolog 7 | 8324 | AGC CAT TTT GTC CTG | CCT TCC TCT TCG TTC |
| | (Drosophilia) | | TTT TC | ACT |
| GARS | Glycyl-tRNA synthetase | 2617 | AGG GAC CGT GAG TCA | AAA CAG AGG ATA CCT |
| | | | A | GGC |
| GATA3 | GATA binding protein 3 | 2625 | AAC TGT CAG ACC ACC | GAA GTC CTC CAG TGA |
| | | | ACA A | GTC AT |
| GRB7 | Growth factor receptor-bound | 2866 | TCG ATG CAC ACA CTG | TTC ACA TCT GCC ACG |
| | protein 7 | | GTA T | TAC T |
| GSTP1 | Glutathione S-transferase pl | 2950 | GGG CTC TAT GGG AAG | GTT CTG GGA CAG CAG |
| | | | G | G |
| HSD1784 | hydroxysteroid (17-beta) | 3295 | TGG GGC TAA GTG GAC | TGC CTT CTG AGG GTC |
| | dehydrogenase 4 | | TAT | AA |
| KIAA0310 | KIAA0310 gene product | 9919 | GCC CTT CTA CAA CCC | GCT CCA AGT GCA AGT |
| | | | TG | TC |
| KIT | V-kit Hardy-Zuckerman 4 feline | 3815 | CAC GCA CCT GCT GAA | TCT ACC ACG GGC TTC |
| | sarcoma viral oncogene homolog | | AT | TGT C |
| KRT17 | Keratin 17 | 3872 | GAG ATT GCC ACC TAC | GAG GAG ATG ACC TTG |
| | | | CG | CC |
| KRT5 | Keratin 5 (epidermolysis bullosa | 3852 | GGA GAA GGA GTT GGA | CCA CTG CTG CTG GAG |
| | simplex, Dowling-Meara/Kobner/ | | CC | TA |
| | Weber-Cockayne types) | | | |
| NAT1 | N-acetyltransferase 1 | 9 | ACA GCA CTC CAG CCA | CTG GTA TGA GCG TCC |
| | (arylamine N-acetyltransferase) | | AA | AAA C |
| PGR | Progesterone receptor | 5241 | AGC TCA CAG CGT TTC | TGT GCA GCA ATA ACT |
| | | | TAT C | TCA GAC |
| PLOD1 | procollagen-lysine | 5351 | CGT GCC GAC TAT TGA | GTA GCG GAC GAC AAA |
| | 1,2-oxoglutarate 5-dioxygenase | | CAT | GG |
| | 1 | | | |
| PTP4A2 | protein tyrosine phosphatase | 8073 | TCA AAG ATT CCA ACG | TCT CAA GTT CCA CTT |
| | type IVA, member 2 | | GTC ATA G | CCA GTA G |
| RABEP1 | Rabaptin-5 | 9135 | ATG TCA GTG AGC AAG | GCT GGT TAA TGT CTG |
| | | | TCC | TCA GT |
| RARRES3 | retinoic acid receptor responder | 5920 | GCT GAG ATA TGG CAA | CTC CTA ATC GCA AAA |
| | (tazarotene induced) 3 | | GTG C | GAG C |
| S100A11 | S100 calcium binding protein A8 | 6282 | CAA AAA TCT CCA GCC | TAA CCA TCC TTT CCA |
| | (calgranulin A) | | CTA CA | GCA TAC |
| SDC2 | Syndecan 2 (heparan sulfate | 6383 | AAA CCA CGA CGC TGA | ATT TGT ATC CTC TTC |
| | proteoglycan 1, cell surface- | | AT | GGC TG |
| | associated, fibroglycan) | | | |
| SLC39A6 | solute carrier family 39 (zinc | 25800 | ACC ACC ATA GTC ATA | CAT ACT TGG ACA ACT |
| | transporter), member 6 | | GCC | GCT TC |
| SLC7A6 | Solute carrier family 7 (cationic | 9057 | AGC GTT TTA CAC CTA | CCA CGA AGA ACC AGT |
| | amino acid transporter, | | TCC C | AGC |
| | y+ system), member 6 | | | |
| SLPI | secretory leukocyte protease | 6590 | GTG TGG GAA ATC CTG | GTG GTG GAG CCA AGT |
| | inhibitor (antileukoproteinase) | | CG | CT |
| SMA3 | SMA3 | 10571 | CCG TAC CTG ATG CAC | GTG CCC GTA GTT GCG |
| | | | GAA | ATA |
| TAP1 | transporter 1, ATP-binding | 6890 | AAG ACA CTC AAC CAG | GGT AGA GAA CAA ATG |
| | cassette, sub-family B (MDR/TAP | | AAG G | TGA CAA GG |
| TRIM29 | Tripartite motif-containing 29 | 23650 | AAC AAC TAC ACG AAC | ATT CTT CTG GGT GGT |
| | | | AGC | CTC |
| XBP1 | X-box binding protein 1 | 7494 | CTG TTG GGC ATT CTG | GGA GGC TGG TAA GGA |
| | | | GAC | ACT |
| Proliferation | | | | |
| genes | | | | |
| BIRC5 | baculoviral IAP repeat-containing 5 | 332 | CGA CCC CAT AGA GGA | TTC TTG ACA GAA AGG |
| | (survivin) | | ACA TAA | AAA GCG |
| BUB1 | budding uninhibited by benzimidazoles | 699 | CAC TTG GGA CTG TTG | TGG ATA GGA ACT CAC |
| | 1 homolog (yeast) | | ATG | TGG T |
| CENPF | Centromere protein F, 350/400 ka | 1063 | CCA CTG AGT CTC GGC | ATT TCG TGG TGG GTT |
| | (mitosin) | | AA | CT |
| CKS2 | CDC28 protein kinase regulatory | 1164 | TGG AGG AGA CTT GGT | GAA TAT GTG GTT CTG |
| | subunit 2 | | GT | GCT CA |
| FAM54A | family with sequence similarity 54, | 113115 | GTG GAA ATG CAG GAA | GCT CGT CAC TCA AGC |
| (=DUFD1) | member A | | CTG AA | CAA |
| GTPBP4 | GTP binding protein 4 | 23560 | GGT GTT GAC ATG GAC | CTT CCC GCT TTC TTT |
| | | | GAT AA | TCC TA |
| HSPA14 | heat shock 70 kDa protein 14 | 51182 | GTT TAG AAG CAA TCA | CCT CCA CAA AGG ACA |
| | | | GAG GAC T | ACC |
| MKI67 | Antigen identified by monocional | 4288 | TCA GAC TCC ATG TGC | CTT CAC TGT CCC TAT |
| | antibody KI-67 | | CT | GAC TTC |
| MYBL2 | v-myb myeloblastosis viral oncogene | 4605 | CAC ACT GCC CAA GTC | AAG CTG TTG TCT TCT |
| | homolog (avian)-like 2 | | TCT A | TTG ATA CC |
| NEK2 | NIMA (never in mitosis gene a)- | 4751 | AGC TTG GAG ACT TTG | GTA ATA AGG TGT GCC |
| | related kinase 2 | | GG | AAC AAA T |
| PCNA | Proliferating cell nuclear antigen | 5111 | GTC ACA GAC AAG TAA | TAC TGA GTG TCA CCG |
| | | | TGT CG | TT |
| STK6 | serine/threonine kinase 6 | 6790 | CTT ACT GTC ATT CGA | ATG CAT CCG ACC TTC |
| | | | AGA GAG TT | AAT C |
| TOP2A | Topolsomerase (DNA) II alpha 170 kDa | 7153 | AAG CAC ATG AGG TGA | TAC CAC AGC CAA TGG |
| | | | AAA AT | CA |
| TTK | TTK protein kinase | 7272 | ACG GAA TCA AGT CTT | TGC CAC TGT TTC TGG |
| | | | CTA GC | TTA C |
| Housekeeper | | | | |
| genes | | | | |
| MRPL19 | Mitochondrial ribosomal protein L19 | 9801 | GGG ATT TGC ATT CAG | GGA AGG GCA TCT CGT |
| | | | AGA TCA G | AAG |
| PSMC4 | Proteasome (prosome, macropain) 26S | 5704 | GGC ATG GAC ATC CAG | CCA CGA CCC GGA TGA |
| | subunit, ATPase, 4 | | AAG | AT |
| PUM1 | Pumilla homolog 1 (Drosophila) | 9698 | TGAGGTGTGCACCATGAAC | CAGAATGTGCTTGCCATAGG |

Real-time PCR. For PCR, each 20 μL reaction included 1×PCR buffer with 3 mM MgCl2 (Idaho Technology Inc., Salt Lake City, Utah), 0.2 mM each of dATP, dCTP, and dGTP, 0.1 mM dTTP, 0.3 mM dUTP (Roche, Indianapolis, Ind.), 10 ng cDNA and 1U Platinum Taq (Invitrogen, Carlsbad, Calif.). The dsDNA dye SYBR Green I (Molecular Probes, Eugene, Oreg.) was used for all quantification (1/50000 final). PCR amplifications were performed on the Lightcycler (Roche, Indianapolis, Ind.) using an initial denaturation step (94° C., 90 sec) followed by 50 cycles: denaturation (94° C., 3 sec), annealing (58° C., 5 sec with 20° C./s transition), and extension (72° C., 6 sec with 2° C./sec transition). Fluorescence (530 nm) from the dsDNA dye SYBR Green I was acquired each cycle after the extension step. Specificity of PCR was determined by post-amplification melting curve analysis. Reactions were automatically cooled to 60° C. at a rate of 3° C./s and slowly heated at 0.1° C./s to 95° C. while continuously monitoring fluorescence.

Relative quantification by RT-PCR. Quantification was performed using the LightCycler 4.0 software. The crossing threshold (Ct) for each reaction was determined using the 2nd derivative maximum method (Wittwer et al. (2004) Washington, D.C.: ASM Press; Rasmussen (2001) Heidelberg: Springer Verlag. 21-34). Relative copy number was calculated using an external calibration curve to correct for PCR efficiency and a within run calibrator to correct for the variability between run. The calibrator is made from 4 equal parts of RNA from 3 cell lines (MCF7, SKBR3, ME16C) and Universal Human Reference RNA (Stratagene, La Jolla, Calif., Cat #740000). Differences in cDNA input were corrected by dividing target copy number by the arithmetic mean of the copy number for 3 housekeeper genes (MRPL19, PSMC4, and PUM1) (Szabo et al. (2004) Genome Biol 5:R59). The normalized relative gene copy number was log2 transformed and analyzed by hierarchical clustering using Cluster (Eisen et al. (1998) Proc Natl Acad Sci USA 95:14863-14868). The clustering was visualized using Treeview software (Eisen Lab, http:/rana.lbl.gov/EisenSoftware.htm).

Microarray experiments. The same 126 samples used for qRT-PCR were analyzed by microarray (Agilent Human oligonucleotide). Total RNA was prepared and quality checked as described above. Labeling and hybridization of RNA for microarray was done using the Agilent low RNA input linear amplification kit (http://www.chem.agilent.com/Scripts/PDS.asp?lPage=10003), but with one-half the recommended reagent volumes and using a Qiagen PCR purification kit to clean up the cRNA. Each sample was assayed versus a common reference sample that was a mixture of Stratagene's Human Universal Reference total RNA (100 ug) enriched with equal amounts of RNA (0.3 μg each) from MCF/and ME16C cell lines. Microarray hybridizations were carried out on Agilent Human oligonucleotide microarrays (1A-v1, 1A-v2 and custom designed 1A-v1 based microarrays) using 2 μg each of Cy3-labeled “reference” and Cy5-labeled “experimental” sample. Hybridizations were done using the Agilent hybridization kit and a Robbins Scientific “22k chamber” hybridization oven. The arrays were incubated overnight and then washed once in 2×SSC and 0.0005% triton X-102 (10 min), twice in 0.1×SSC (5 min), and then immersed into Agilent Stabilization and Drying solution for 20 seconds. All microarrays were scanned using an Axon Scanner 4000A. The image files were analyzed with GenePix Pro 4.1 and loaded into the UNC Microarray Database at the University of North Carolina at Chapel Hill (https://genome.unc.edu/) where a lowess normalization procedure was performed to adjust the Cy3 and Cy5 channels (Yang et al. (2002) Nucleic Acids Res 30:e15). All primary microarray data associated with this study are available at the UNC Microarray Database and have been deposited into the GEO (http://www.ncbi.nlm.nih.gov/geo/) under the accession number of GSE1992, series GSM34424-GSM34568.

Selecting genes for real-time qRT-PCR. A new “intrinsic” gene set for classifying breast tumors was derived using 45 before and after therapy samples from the combined data sets presented in Sorlie et al. (see Table 9 for the list of 45 pairs). The two-color DNA microarray data sets were downloaded from the internet and the R/G ratio (experimental/reference) for each spot was normalized and log2 transformed. Missing values were imputed using the k-NN imputation algorithm described by Troyanskaya et al. (Troyanskaya et al. (2001) Bioinformatics 17:520-525). The “intrinsic” analysis identified 550 gene elements.

45 Paired Samples for Intrinsic Analysis from Sorlie et al. 2003

shaz111.BC.FUMI05.AF
shaz110.BC.FUMI05.BE
shaz105.BC.FUMI06.AF
shaz104.BC.F, UMI06.BE
shaz117.BC.FUMI07.AF
shaz116.BC.FUMI07.BE
shby032.BC.FUMI20.AF
shby020.BC.FUMI20.BE
shaz123.BC.FUMI27.AF
shaz122.BC.FUMI27.BE
shaz115.BC.FUMI35.AF
shaz114.BC.FUMI35.BE
shaz127.BC.FUMI37.AF
shaz126.BC.FUMI37.BE
svl012..BC104A.BE
svl013..BC104B.AF
svl005..BC106A.AF
svl006..BC106B.BE
svcc63..BC107A.AF
svcc98..BC107B.BE
svl003..BC108A.BE
svl004..BC108B.AF
svcc77..BC110A.AF
svcc78..BC110B.BE
svcc97..BC112A.AF
svcc53..BC112B.BE
svcc81..BC114A.BE
svcc52..BC114B.AF
svcc64..BC115A.AF
svcc106.BC115B.BE
svcc112.BC118A.AF
svcc134.BC118B.BE
svl015..BC119A.BE
svl014..BC119B.AF
svl027..BC120A.BE
svl028..BC120B.AF
svl017..BC121A.AF
svl016..BC121B.BE
svcc91..BC123A.AF
svcc89..BC123B.BE
svcc111.BC124A.BE
svcc109.BC124B.AF
svl018..BC125A.BE
svl019..BC125B.AF
svcc96..BC2
svcc113.BC2.LN2
svcc93..BC206A.BE
svcc135.BC206B.AF
svcc107.BC208A.BE
svcc125.BC208B.AF
svcc79..BC213A.AF
svcc76..BC213B.BE
svcc103.BC214A.AF
svcc92..BC214B.BE
svl021..BC303A.AF
svl020..BC303B.BE
svcc131.BC305A.BE
svcc58..BC305B.AF
svl032..BC307A.AF
svl103..BC307B.BE
svcc115.BC38
svcc116.BC38.LN38
svcc66..BC402B.AF
svcc83..BC402B.BE
svcc36..BC404A.AF
svl033..BC404B.BE
svl029..BC405A.BE
svl030..BC405B.AF
shby035.BC601A.BE
shby036.BC601B.AF
svl042..BC608A.AF
svl036..BC608B.BE
svl040..BC702A.AF
svl041..BC702B.BE
shby034.BC703A.AF
shby037.BC703B.BE
svl039..BC706A.BE
svl038..BC706B.AF
svcc86..BC708A.AF
svcc104.BC708B.BE
svcc85..BC709A.AF
svcc84..BC709B.BE
svcc101.BC710A.BE
svcc82..BC710B.AF
svcc65..BC711A.AF
svcc120.BC711B.BE
svcc105.BC805A.BE
svcc121.BC805B.AF
svcc126.BC808A.AF
svcc124.BC808A.BE.

Next, a completely independent data set was utilized (van't Veer et al. 2002) to derive an optimized version of the 550 intrinsic gene list. To allow across data set analyses, gene annotation from each dataset was translated to UniGene Cluster IDs (UCID) using the SOURCE database (Diehn et al. (2003) Nucleic Acids Res 31:219-223). Following the algorithm outlined by Tibshirani and colleagues (Bair et al. (2004) PLoS Biol 2:E108; Bullinger et al. (2004) N Engl J Med 350:1605-1616), the 97 samples from the van't Veer et al. 2002 study were hierarchical clustered using a common set of 350 genes, and assigned an “intrinsic subtype of either Luminal, HER2+/ER−, Basal-like, or Normal-like to each sample. A feature/gene selection was then performed to identify genes that optimally distinguished these 4 classes using a version of the gene selection method first described by Dudoit et al. (Genome Biol 3:RESEARCH0036), where the best class distinguishers are identified according to the ratio of between-group to within-group sums of squares. In addition to statistically selecting “intrinsic” classifiers proliferation genes (e.g., TOP2A, KI-67, PCNA) were also chosen, and other important prognostic markers (e.g., PgR) that have potential for diagnostics. In total, 53 differentially expressed biomarkers were used in the real-time qRT-PCR assay (Table 8).

Combining microarray and qRT-PCR datasets. Distance Weighted Discrimination (DWD) was used to identify and correct systematic biases across the microarray and qRT-PCR datasets (Benito et al. (2004) Bioinformatics 20:105-114). Prior to DWD, each dataset was normalized by setting the mean to zero and the variance to one. Normalization was done within each microarray experiment and for genes profiled across many experimental runs for real-time qRT-PCR. After DWD, genes in common between the datasets were clustered using Spearman correlation and average linkage association.

Receiver operator curves. In order to determine agreement between protein expression (immunohistochemistry) and gene expression (qRT-PCR), a cut-off for relative gene copy number was selected by minimizing the sum of the observed false positive and false negative errors. That is, minimizing the estimated overall error rate under equal priors for the presence/absence of the protein. The sensitivity and specificity of the resulting classification rule were estimated via bootstrap adjustment for optimism (Efron et al. (1998) CRC Press LLC. p 247 pp).

Survival analyses. Survival curves were estimated by the Kaplan-Meier method and compared via a log-rank or stratified log-rank test as appropriate. Standard clinical pathological parameters of age (in years), node status (positive vs. negative), tumor size (cm, as a continuous variable), grade (1-3, as a continuous covariate), and ER status (positive vs. negative) were tested for differences in RFS and OS using Cox proportional hazards regression model. Pairwise log-rank tests were used to test for equality of the hazard functions among the intrinsic classes. Only the classes Luminal, HER2+/ER−, and Basal-like classes were included in the analyses because it was believed the Normal Breast-like subtype is not a pure tumor class and may result from normal breast contamination. Cox regression was used to determine predictors of survival from continuous expression data. All statistical analyses were performed using the R statistical software package (R Foundation for Statistical Computing).

b) Results

Recapitulating microarray breast cancer classifications by qRT-PCR. 126 different breast tissue samples (117 invasive, 5 normal, 1 fibroadenoma, and 3 cell lines) were expression profiled using a real-time qRT-PCR assay comprised of 53 biological classifiers and 3 control/housekeepers genes. Genes were statistically selected to optimally identify the 4 main breast tumor intrinsic subtypes, and to create an objective gene expression predictor for cell proliferation and outcome (Ross et al. (2000) Nat Genet. 24:227-235).

There were 402 genes in common between this microarray dataset and the 550 “intrinsic” genes selected from the Sorlie et al. 2003 study. Two-way hierarchical clustering of the 402 genes in the microarray gave the same tumor subtypes as the minimal 37 “intrinsic” genes assayed by qRT-PCR (FIG. 4). The samples were grouped into Luminal, HER2+/ER−, Normal-like, and Basal-like subtypes. Out of 123 breast samples compared across the platforms, 114 (93%) were classified the same. The minimal “intrinsic” gene set identified expression signatures within the 3 different cell lines that were characteristic of each tumor subtype: Luminal (MCF7), HER2+/ER− (SKBR3), and Basal-like (ME16C). The genes EGFR and PgR, which were added for their predictive and prognostic value in breast cancer Nielsen et al. (2004) Clin Cancer Res 10:5367-5374; Makretsov et al. (2004) Clin Cancer Res 10:6143-6151), had opposite expression and were found to associate with either ER-positive tumors (high expression of PgR) or ER-negative tumors (high expression of EGFR) (FIG. 4C).

Proliferation and grade. Expression of the 14 “proliferation” genes (FIG. 4D) assayed by qRT-PCR showed that Luminal tumors have relatively low replication activity compared to HER2+/ER− and Basal-like tumors. As expected, the Normal-like samples showed the lowest expression of the “proliferation” genes. When correlating (Spearman correlation) the gene expression of all 53 genes with grade, it was found that the top 3 proliferation genes with a positive correlation (i.e., high expression correlates with high grade) were the proliferation genes CENPF (p=2.00E-07), BUB1 (p=6.84E-07), and STK6 (p=2.67E-06) (see supplementary Table 10). Interestingly, all the proliferation genes, except PCNA, were at the top of the list for having a positive correlation to grade. Conversely, the top markers with significant negative correlations with grade (i.e., low expression correlates with high grade) were GATA3 (p=3.53E-07), XBP1 (p=9.64E-06), and ESR1 (p=4.53E-05).

Agreement between immunohistochemistry, qRT-PCR “intrinsic” classifications, and gene expression. Fifty out of fifty-five (91%) Luminal tumors with IHC data were scored positive for ER. Conversely, 50 out of 56 (89%) tumors classified as HER2+/ER− or Basal-like were negative for ER by IHC. Cluster analysis showed that the Luminal tumors co-express ER and estrogen responsive genes such as LIV1/SLC39A6, X-box binding protein 1 (XBP1), and hepatocyte nuclear factor 3a (HNF3A/FOXA1). The gene with the highest correlation in expression to ESR1 was GATA3 (0.79, 95% CI: 0.71-0.85). It was found that the gene expression of ESR1 alone had 88% sensitivity and 85% specificity for calling ER status by IHC, and GATA3 alone showed 79% sensitivity and 88% specificity (FIG. 5A). In addition, gene expression of PgR correlated well with PR IHC status (sensitivity=89%, specificity=82%) (FIG. 5B). The data showed a very high correlation in expression between HER2/ERBB2 and GRB7 (0.91, 95% CI: 0.87-0.94), which are physically located near one another and are commonly overexpressed and DNA amplified together (Pollack et al. (1999) Nature Genetics 23:41-46; Pollack et al. (2002) Proc Natl Acad Sci USA 99:12963-12968). However, neither ERBB2 (sensitivity=91%, specificity=54%) nor GRB7 (sensitivity=52%, specificity=78%) gene expression had both high sensitivity and specificity for predicting HER2 status by IHC (FIG. 5C).

Reproducibility of qRT-PCR. The run-to-run variation in Cp (cycle number determined from fluorescence crossing point) for all 56 genes (53 classifiers and 3 housekeepers) was determined from 8 runs. The median CV (standard deviation/mean) for all the genes was 1.15% (0.28%-6.55%) and 51/56 genes (91%) had a CV 7%. The reproducibility of the classification method is illustrated from the observation that replicates of the same sample (UB57A&B and UB60A&B), cluster directly adjacent to one another. Notably, the replicates were from separate RNA/cDNA preparations done on different pieces of the same tumor.

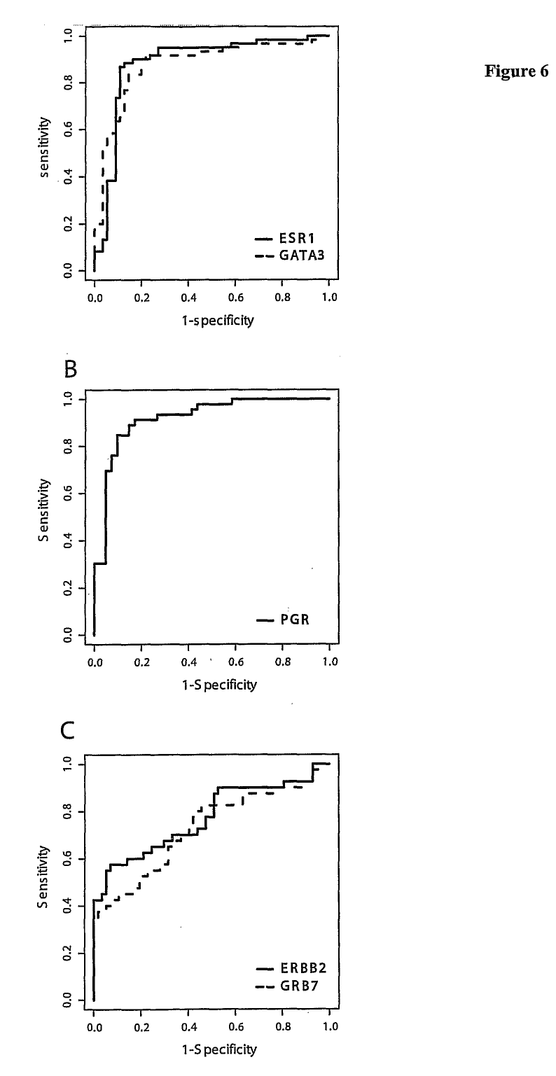

Survival Predictors. The clinical significance of individual markers and “intrinsic” subtypes were analyzed using qRT-PCR data. Patients with Luminal tumors showed significantly better outcomes for relapse-free survival (RFS) and overall survival (OS) compared to HER2+/ER− (RFS: p=0.023; OS: p=0.003) and Basal-like (RFS: p=0.065; OS: p=0.002) tumors (FIG. 6). This difference in outcome was significant for overall survival even after adjustment for stage (HER2+/ER−: p=0.043; Basal-like: p=0.001). There was no difference in outcome between patients with HER2+/ER− and Basal-like tumors. Analysis of the same cohort using standard clinical pathological information shows that stage, tumor size, node status, and ER status were prognostic for RFS and OS.

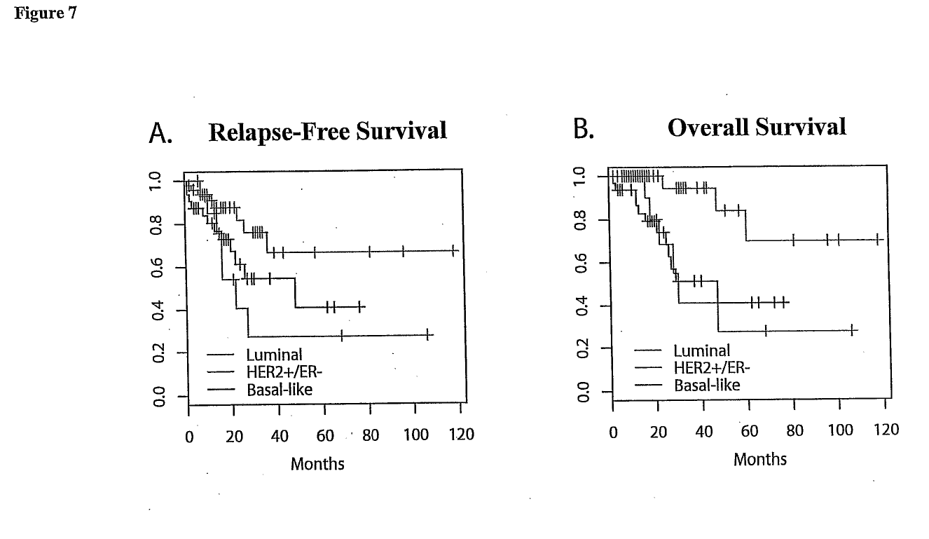

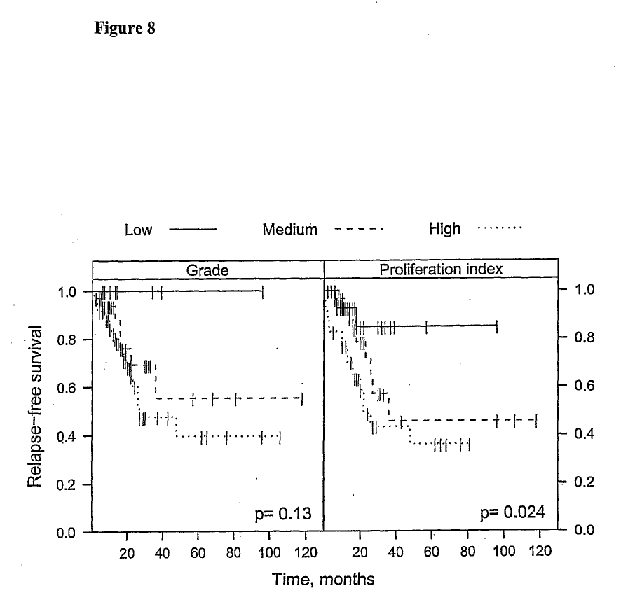

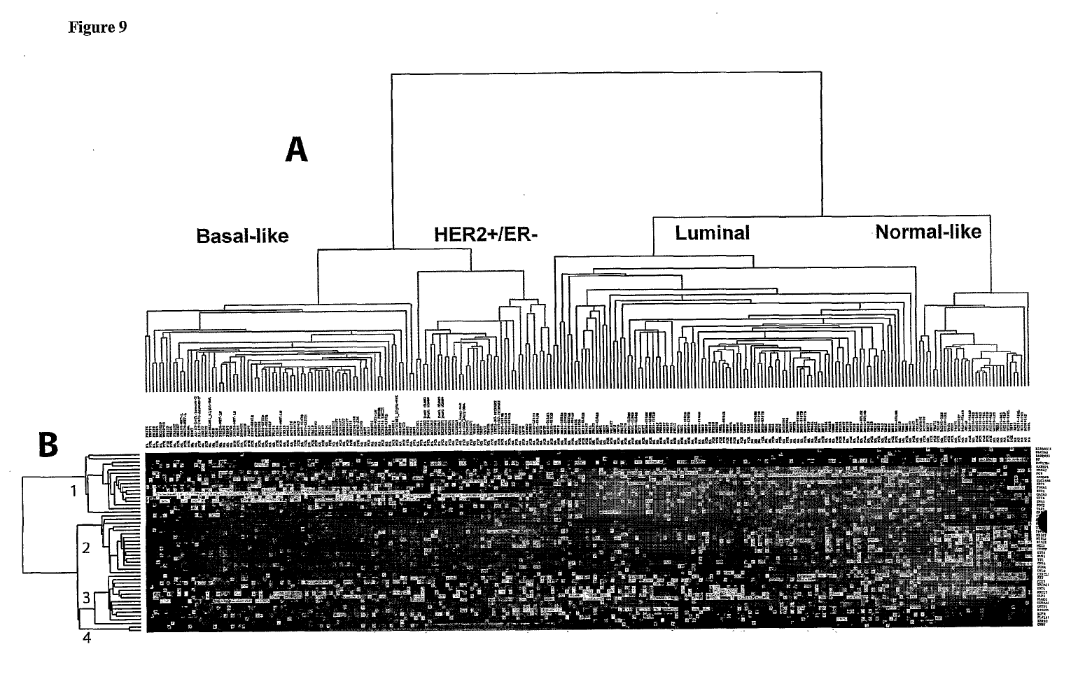

Using a Cox proportional hazards model to find biomarkers from the qRT-PCR data that predict survival, it was found that high expression of the proliferation genes GTBP4 (p=0.011), HSPA14 (p=0.023), and STK6 (p=0.027) were significant predictors of RFS independent of grade and stage (FIG. 7). The only proliferation gene significant for OS after correction for grade and stage was GTBP4 (p=0.011). Overall, the best predictor for both RFS (p=0.004) and OS (p=0.004) independent of grade and stage was SMA3 (Table 10).

TABLE 10

| Gene | OS˜Gene | OS˜Gene + Grade | OS˜Gene + Stage | OS˜Gene + Grade + Stage | Prolif. Gene |
|---|---|---|---|---|---|
| SMA3 | 0.0010086 | 0.00814571 | 0.000398174 | 0.00357674 | NO |
| KIT | 0.000332738 | 0.00154407 | 0.00272027 | 0.00672142 | NO |
| GTPBP4 | 0.00445804 | 0.0307721 | 0.00150072 | 0.0112402 | YES |
| COX6C | 0.00289023 | 0.00951953 | 0.0028745 | 0.0125619 | NO |
| CX3CL1 | 0.00217324 | 0.00425494 | 0.0181299 | 0.0152864 | NO |
| KRT17 | 0.0321012 | 0.0420179 | 0.0233713 | 0.015837 | NO |
| B3GNT5 | 0.032762 | 0.117857 | 0.00427977 | 0.02214 | NO |
| PLOD | 0.00730183 | 0.0152132 | 0.052899 | 0.0406316 | NO |
| SLPI | 0.0533249 | 0.0795638 | 0.0372877 | 0.0608959 | NO |
| DSC2 | 0.0432628 | 0.19777 | 0.0199733 | 0.0720347 | NO |
| GRB7 | 0.0023925 | 0.00997476 | 0.0212037 | 0.076893 | NO |
| TRIM29 | 0.0758398 | 0.0969003 | 0.10943 | 0.0808424 | NO |
| STK6 | 0.0353601 | 0.192395 | 0.0169665 | 0.0990307 | YES |
| BUB1 | 0.0572953 | 0.237675 | 0.0218123 | 0.123044 | YES |
| NAT1 | 0.0127223 | 0.0791954 | 0.0189787 | 0.135405 | NO |
| CYB5 | 0.0557461 | 0.287241 | 0.0273843 | 0.137872 | NO |
| PTP4A2 | 0.160424 | 0.0858591 | 0.342854 | 0.138471 | NO |
| TTK | 0.110921 | 0.45438 | 0.0192107 | 0.143497 | YES |
| HSPA14 | 0.391113 | 0.8142 | 0.0511814 | 0.144083 | YES |
| GATA3 | 0.0324598 | 0.289619 | 0.0175668 | 0.157456 | NO |
| ESR1 | 0.030409 | 0.145509 | 0.0405537 | 0.184542 | NO |
| SLC39A6 | 0.0733459 | 0.430962 | 0.024724 | 0.207555 | NO |
| ERBB2 | 0.0459011 | 0.0828308 | 0.169867 | 0.24427 | NO |
| FOXA1 | 0.110671 | 0.4427 | 0.094167 | 0.330446 | NO |
| EGFR | 0.145898 | 0.183089 | 0.3197 | 0.357336 | NO |
| DUFD1 | 0.378603 | 0.985614 | 0.0888335 | 0.359478 | YES |
| MYBL2 | 0.0399249 | 0.176578 | 0.0716375 | 0.361422 | YES |
| S100A11 | 0.34613 | 0.556875 | 0.230849 | 0.363064 | NO |
| XBP1 | 0.045776 | 0.268606 | 0.0926021 | 0.400871 | NO |
| TOP2A | 0.240971 | 0.655786 | 0.0969129 | 0.404568 | YES |
| KIAA0310 | 0.484382 | 0.772587 | 0.342042 | 0.406749 | NO |
| KRT5 | 0.985088 | 0.984712 | 0.641471 | 0.409027 | NO |
| BF | 0.046196 | 0.204647 | 0.105472 | 0.463932 | NO |
| GSTP1 | 0.687906 | 0.677131 | 0.557251 | 0.465849 | NO |
| FZD7 | 0.594194 | 0.90597 | 0.384141 | 0.47759 | NO |
| NEK2 | 0.46014 | 0.932809 | 0.172718 | 0.500592 | YES |
| TAP1 | 0.663093 | 0.482788 | 0.541857 | 0.534398 | NO |
| FLJ14525 | 0.17537 | 0.17907 | 0.613531 | 0.561022 | NO |
| ACADSB | 0.0698192 | 0.387308 | 0.118621 | 0.576123 | NO |
| GARS | 0.709987 | 0.923267 | 0.902252 | 0.630522 | NO |
| BIRC5 | 0.397737 | 0.975853 | 0.170876 | 0.632892 | YES |
| HSD17B4 | 0.206242 | 0.395994 | 0.305472 | 0.635554 | NO |
| MKI67 | 0.311764 | 0.709371 | 0.195635 | 0.640833 | YES |
| PCNA | 0.868635 | 0.731512 | 0.557926 | 0.645851 | YES |
| PGR | 0.355079 | 0.965257 | 0.181127 | 0.681739 | NO |
| RABEP1 | 0.543773 | 0.963589 | 0.377702 | 0.682359 | NO |
| SLC7A6 | 0.432451 | 0.689547 | 0.419107 | 0.685462 | NO |
| SDC2 | 0.47607 | 0.37331 | 0.914923 | 0.689713 | NO |
| CKS2 | 0.936337 | 0.36756 | 0.180917 | 0.763492 | YES |
| DP1 | 0.149164 | 0.576409 | 0.32648 | 0.839276 | NO |
| CENPF | 0.19591 | 0.730895 | 0.203913 | 0.8435 | YES |
| CDK2AP1 | 0.711736 | 0.908545 | 0.835195 | 0.883836 | NO |
| RARRES3 | 0.0189691 | 0.107372 | 0.398642 | 0.943889 | NO |

Co-clustering qRT-PCR and Microarray Data. In order to determine if qRT-PCR and microarray data could be analyzed together in a single dataset, DWD was used to combine data for 50 genes and 126 samples profiled on both platforms (252 samples total). Hierarchical clustering of these data show that 98% (124/126) of the paired samples classified in the same group and 83/126 (66%) clustered directly adjacent to their corresponding partner (FIG. 10). Thus, DNA microarray and real-time qRT-PCR can be combined into a seamless dataset without sample segregation based on platform. Overall, the correlation between microarray and qRT-PCR expression data was 0.76 (95% CI: 0.75, 0.77) before DWD and 0.77 (95% CI: 0.76, 0.78) after DWD (FIG. 5). The DWD does not significantly effect the correlation but corrects for systematic biases between the platforms.

c) Discussion

Gene expression analyses can identify differences in breast cancer biology that are important for prognosis. However, a major challenge in using genomics for diagnostics is finding biomarkers that can be reproducibly measured across different platforms and that provide clinically significant classifications on different patient populations. Using microarray data, 402 “intrinsic” genes were identified that classify breast cancers based on vastly different expression patterns. This “intrinsic” gene set was shown to provide the same classifications when applied to a completely new and ethnically diverse population. Furthermore, the microarray dataset can be minimized to 37 “intrinsic” genes, translated into a real-time qRT-PCR assay, and provide the same classifications as the larger gene set. Molecular classifications using the “intrinsic” qRT-PCR assay agree with standard pathology and are clinically significant for prognosis. Thus, biological classifications based on “intrinsic” genes are robust, reproducible across different platforms, and can be used for breast cancer diagnostics.

The greatest contribution genomic assays have made towards clinical diagnostics in breast cancer has been in identifying risk of recurrence in women with early stage disease. For instance, MammaPrint™ is a microarray assay based on the 70 gene prognosis signature originally identified by van't Veer et al. On the test set validation, the 70 gene assay found that individuals with a poor prognostic signature had approximately a 50% chance of remaining free of distant metastasis at 10 years while those with a good-prognostic signature had a 85% chance of remaining free of disease. Another assay with similar utility is Oncotype Dx (Genomic Health Inc)—a real-time qRT-PCR assay that uses 16 classifiers to assess if patients with ER positive tumors are at low, intermediate, or high risk for relapse. While recurrence can be predicted with high and low risk tumors, patients in the intermediate risk group still have variable outcomes and need to be diagnosed more accurately.

In general, tumors that have a low risk of early recurrence are low grade and have low expression of proliferation genes. Due to the correlation of proliferation genes with grade and their significance in predicting outcome, a group of 14 proliferation genes were assayed. While the classic proliferation markers TOP2A and MKI67 significantly correlated with grade in the cohort, they were not near the top of the list. Furthermore, PCNA did not significantly correlate with grade (p=0.11) in the cohort. This could result from PCR primer design or differences between RNA and protein stability. Nevertheless, the proliferation gene that was found had the highest correlation to grade was CENPF (mitosin); another commonly used mitotic marker that has been shown to correlate with grade and outcome in breast cancer (Clark et al. (1997) Cancer Res 57:5505-5508). Since tumor grade and the mitotic index have been shown to be important in predicting risk of relapse (Chia et al. (2004) J Clin Oncol 22:1630-1637; Manders et al. (2003) Breast Cancer Res Treat 77:77-84), it is not surprising that 4 (GTBP4, HSPA14, STK6/15, BUB1) out the top 5 predictors for RFS (independent of stage) were proliferation genes. The proliferation gene that was the best predictor of RFS was GTBP4, a GTP-binding protein implicated in chronic renal disease and shown to be upregulated after serum administration (i.e., serum response gene) (Laping et al. (2001) J Am Soc Nephrol 12:883-890). Overall, the best predictor for both RFS (p=0.004) and OS (p=0.004) independent of grade and stage was SMA3. The role of SMA3 in the pathogenesis of breast cancer is still unclear, although it has also been associated with the BCL2 anti-apoptotic pathway (Iwahashi et al. (1997) Nature 390:413-417).

### 3. Example 3

### Ewing's Sarcoma

The test disclosed herein is able to detect the most common types of EWS-FLI1 translocations that occur in the Ewing's sarcoma family of tumors, distinguishes between the EWS-FLI1 type 1 and type 2 fusions, and use real-time RT-PCR with dual-labeled probes specific for EWS-FLI1 translocations

Tumors classified in the Ewing's family (Ewing's sarcoma, PNET, and Askin's sarcoma) are the most common malignant bone and soft tissue tumors occurring in childhood and young adulthood. By light microcopy, it is sometimes difficult to differentiate tumors within the Ewing's family from each other and from other small round cell tumors. Accurate diagnosis of the tumor type is essential for prognosis and determining therapy. Real-time RT-PCR can be used to identity specific tumor types within the Ewing's family by the detection of characteristic translocations. The two most common types of translocations in the Ewing's family of tumors are the EWS/FLI1 gene fusion (t(11:22)(q24;q12)) and the EWS/ERG gene fusion (t(21;22)(q22;q12)). Both these translocations are diagnostic for Ewing's sarcoma. Other chimeric genes have been observed on a rare basis in Ewing's sarcoma, including EWS/ETV1 (t(7:22), EWS/E1AF (t(17;22)), and EWS/FEV (t(2;22)).

The EWS/FLI1 fusion transcripts occur in several forms. The type 1 transcript is the most common (65% of cases), and is created by the fusion of the EWS exons 1-7 to FLI1 exons 6-9. The type 2 translocation results from EWS exons 1-7 joining to exons 5-9 of FLI1 and is seen in approximately 25% of EWS/FLI1 cases.

This assay can be used to confirm the histological diagnosis of Ewing's sarcoma by detection of either the type 1 or type 2 EWS/FLI1 translocations. A negative result does not exclude the diagnosis of Ewing's sarcoma or other tumor (s—delete the s) types in the Ewing's family since other transcripts (e.g., EWS/ERG) can also define the disease.

A positive EWS/FLI1 gene fusion is reported when an amplification curve is present in the EWS-FLI1 assay (testing for the presence of type 1 and type 2 fusions) and the MRPL19 control assay. A negative EWS/FLI1 result is reported when there is amplification of the control gene (MRPL19) but no transcript specific amplification for either the type 1 or type 2 EWS/FLI1 fusions.

This assay detects and distinguishes between the EWS/FLI type 1 and type 2 gene fusions, which are found in the majority of Ewing's sarcomas. RNA from patient samples and controls is extracted and reverse transcribed using gene specific primers for the EWS/FLI1 fusion and the MRPL19 control gene. The cDNA is then PCR amplified for the EWS/FLI1 fusion and MRPL19 gene in the presence of fluorescently labeled sequence specific probes. Amplification of the control gene and each fusion type is done in separate reactions (i.e., not multiplexed).

Fluorescent in situ hybridization (FISH) is a technique that utilizes fluorescently labeled DNA probes to detect alterations within the genome. The test requires manual interpretation of the FISH signal from 100 cells. A positive result for Ewing's sarcoma is reported when there are chromosome 22q12 rearrangements or break-aparts observed in 25 percent or more of the cells counted.

### G. REFERENCES

Akilesh S, Shaffer D J, Roopenian D. “Customized molecular phenotyping by quantitative gene expression and pattern recognition analysis” Genome Res 13:1719-1727 (2003).
Bair, E., and Tibshiralii, R. “Semi-supervised methods to predict patient survival from gene expression data” PLoS Biol 2:E108 (2004).
Bloom, H. J. G., and Richardson, W. W. “Histologic grading and prognosis in breast cancer” British Journal of Cancer 9:359-377 (1957).
Benito, M., Parker, J., Du, Q., Wu, J., Xiang, D., Perou, C. M., and Marron, J. S. “Adjustment of systematic microarray data biases” Bioinformatics 20:105-114 (2004).
Bhatia P, Taylor W R, Greenberg A H, Wright J A. “Comparison of glyceraldehyde-3-phosphate dehydrogenase and 28S-ribosomal RNA gene expression as RNA loading controls for northern blot analysis of cell lines of varying malignant potential” Anal Biochem 216:223-226 (1994).
Bullinger, L., Dohner, K., Bair, E., Frohling, S., Schlenk, R. F., Tibshirani, R., Dohner, H., and Pollack, J. R. “Use of gene-expression profiling to identify prognostic subclasses in adult acute myeloid leukemia” N Engl J Med 350:1605-1616 (2004).
Buzdar, A., O'Shaughnessy, J. A., Booser, D. J., Pippen, J. E., Jr., Jones, S. E., Munster, P. N., Peterson, P., Melemed, A. S., Winer, E., and Hudis, C. “Phase II, randomized, double-blind study of two dose levels of arzoxifene in patients with locally advanced or metastatic breast cancer” J Clin Oncol 21:1007-1014 (2003).
Caly, M., Genin, P., Ghuzlan, A. A., Elie, C., Freneaux, P., Klijanienko, J., Rosty, C., Sigal-Zafrani, B., Vincent-Salomon, A., Douggaz, A., et al. “Analysis of correlation between mitotic index, MIB1 score and S-phase fraction as proliferation markers in invasive breast carcinoma. Methodological aspects and prognostic value in a series of 257 cases” Anticancer Res 24:3283-3288 (2004).
Chia, S. K., Speers, C. H., Bryce, C. J., Hayes, M. M., and Olivotto, I. A. “Ten-year outcomes in a population-based cohort of node-negative, lymphatic, and vascular invasion-negative early breast cancers without adjuvant systemic therapies” J Clin Oncol 22:1630-1637 (2004).
Clark, G. M., Allred, D. C., Hilsenbeck, S. G., Chamness, G. C., Osborne, C. K., Jones, D., and Lee, W. H. “Mitosin (a new proliferation marker) correlates with clinical outcome in node-negative breast cancer” Cancer Res 57:5505-5508 (1997).
Cronin, M., Pho, M., Dutta, D., Stephans, J. C., Shak, S., Kiefer, M. C., Esteban, J. M., and Baker, J. B. “Measurement of gene expression in archival paraffin-embedded tissues: development and performance of a 92-gene reverse transcriptase-polymerase chain reaction assay” Am J Pathol 164:35-42 (2004).
Dalton, L. W., Page, D. L., and Dupont, W. D. “Histologic grading of breast carcinoma. A reproducibility study” Cancer 73:2765-2770 (1994).
Dhanasekaran S M, Barrette T R, Ghosh D, Shah R, Varambally S, Kurachi K, Pienta K J, Rubin M A, Chinnaiyan A M. “Delineation of prognostic biomarkers in prostate cancer” Nature 412:822-826 (2001).
Diehn, M., Sherlock, G., Binkley, G., Jin, H., Matese, J. C., Hernandez-Boussard, T., Rees, C. A., Cherry, J. M., Botstein, D., Brown, P. O., et al. “SOURCE: a unified genomic resource of functional annotations, ontologies, and gene expression data” Nucleic Acids Res 31:219-223 (2003).
Dudoit, S., and Fridlyand, J. “A prediction-based resampling method for estimating the number of clusters in a dataset” Genome Biol 3:RESEARCH0036 (2002).
Efron, B., Tibshirani, R. J. “An Introduction to the Bootstrap” Boca Raton, Fla.: CRC Press LLC. p 247 pp (1998).
Eggert A, Brodeur G M, Ikegaki N. “Relative quantitative RT-PCR protocol for TrkB expression in neuroblastoma using GAPD as an internal control” Biotechniques 28:681-682, 686, 688-691 (2000).
Eisen, M. B., Spellman, P. T., Brown, P. O., and Botstein, D. “Cluster analysis and display of genome-wide expression patterns” Proc Natl Acad Sci USA 95:14863-14868 (1998).
Elston, C. W., and Ellis, I. O. “Pathological prognostic factors in breast cancer. I. The value of histological grade in breast cancer: experience from a large study with long-term follow-up” Histopathology 19:403-410 (1991).
Fisher, E. R., Osborne, C. K., McGuire, W. L., Redmond, C., Knight, W. A., 3rd, Fisher, B., Bannayan, G., Walder, A., Gregory, E. J., Jacobsen, A., et al. “Correlation of primary breast cancer histopathology and estrogen receptor content” Breast Cancer Res Treat 1:37-41 (1981).
Fisher, B., Costantino, J., Redmond, C., Poisson, R., Bowman, D., Couture, J., Dimitrov, N. V., Wolmark, N., Wickerham, D. L., Fisher, E. R., et al. “A randomized clinical trial evaluating tamoxifen in the treatment of patients with node-negative breast cancer who have estrogen-receptor-positive tumors” N Engl J Med 320:479-484 (1989).
Fitzgibbons, P. L., Page, D. L., Weaver, D., Thor, A. D., Allred, D. C., Clark, G. M., Ruby, S. G., O'Malley, F., Simpson, J. F., Connolly, J. L., et al. “Prognostic factors in breast cancer. College of American Pathologists Consensus Statement 1999” Arch Pathol Lab Med 124:966-978 (2000).
Frank S G, Bernard, P. S. “Profiling Breast Cancer using Real-Time Quantitative PCR. In Rapid Cycle Real-Time PCR: Methods and Applications” Edited by S. Meuer W, C., Nakagawara, K. Heidelberg, Germany, Springer pp 95-106 (2003).
Frierson, H. F., Jr., Wolber, R. A., Berean, K. W., Franquemont, D. W., Gaffey, M. J., Boyd, J. C., and Wilbur, D. C. “Interobserver reproducibility of the Nottingham modification of the Bloom and Richardson histologic grading scheme for infiltrating ductal carcinoma” Am J Clin Pathol 103:195-198 (1995).
Genestie, C., Zafrani, B., Asselain, B., Fourquet, A., Rozan, S., Validire, P., Vincent-Salomon, A., and Sastre-Garau, X. “Comparison of the prognostic value of Scarff-Bloom-Richardson and Nottingham histological grades in a series of 825 cases of breast cancer: major importance of the mitotic count as a component of both grading systems” Anticancer Res 18:571-576 (1998).
Greenough, R. B. “Varying degrees of malignancy in cancer of the breast” J Cancer Res 9:452-463 (1925).
Gruvberger, S., Ringner, M., Chen, Y., Panavally, S., Saal, L. H., Borg, A., Ferno, M., Peterson, C., and Meltzer, P. S. “Estrogen receptor status in breast cancer is associated with remarkably distinct gene expression patterns” Cancer Res 61:5979-5984 (2001).
Henson, D. E., Ries, L., Freedman, L. S., and Carriaga, M. “Relationship among outcome, stage of disease, and histologic grade for 22,616 cases of breast cancer. The basis for a prognostic index” Cancer 68:2142-2149 (1991).
Ishida, S., Huang, E., Zuzan, H., Spang, R., Leone, G., West, M., and Nevins, J. R. “Role for E2F in control of both DNA replication and mitotic functions as revealed from DNA microarray analysis” Mol Cell Biol 21:4684-4699 (2001).
Iwahashi, H., Eguchi, Y., Yasuhara, N., Hanafasa, T., Matsuzawa, Y., and Tsujimoto, Y. “Synergistic anti-apoptotic activity between Bcl-2 and SMN implicated in spinal muscular atrophy” Nature 390:413-417 (1997).
Kollias, J., Murphy, C. A., Elston, C. W., Ellis, I. O., Robertson, J. F., and Blamey, R. W. “The prognosis of small primary breast cancers” Eur J Cancer 35:908-912 (1999).
Kristt D, Turner I, Koren R, Ramadan E, Gal R. “Overexpression of cyclin D1 mRNA in colorectal carcinomas and relationship to clinicopathological features: an in situ hybridization analysis” Pathol Oncol Res 6:65-70 (2000).
Laping, N. J., Olson, B. A., and Zhu, Y. “Identification of a novel nuclear guanosine triphosphate-binding protein differentially expressed in renal disease” J Am Soc Nephrol 12:883-890 (2001).
Manders, P., Bult, P., Sweep, C. G., Tjan-Heijnen, V. C., and Beex, L. V. “The prognostic value of the mitotic activity index in patients with primary breast cancer who were not treated with adjuvant systemic therapy” Breast Cancer Res Treat 77:77-84 (2003).
Makretsov, N. A., Huntsman, D. G., Nielsen, T. O., Yorida, E., Peacock, M., Cheang, M. C., Dunn, S. E., Hayes, M., van de Rijn, M., Bajdik, C., et al. “Hierarchical clustering analysis of tissue microarray immunostaining data identifies prognostically significant groups of breast carcinoma” Clin Cancer Res 10:6143-6151 (2004).
Michels, J. J., Maniay, J., Delozier, T., Denoux, Y., and Chasle, J. “Proliferative activity in primary breast carcinomas is a salient prognostic factor” Cancer 100:455-464 (2004).
Miller C L, Yolken R H. “Methods to optimize the generation of cDNA from postmortem human brain tissue” Brain Res Brain Res Protoc 10:156-167 (2003).
Mischel P S, Nelson S F, Cloughesy T F. “Molecular analysis of glioblastoma: pathway profiling and its implications for patient therapy” Cancer Biol Ther 2:242-247 (2003).
Nielsen, T. O., Hsu, F. D., Jensen, K., Cheang, M., Karaca, G., Hu, Z., Hernandez-Boussard, T., Livasy, C., Cowan, D., Dressler, L., et al. “Immunohistochemical and clinical characterization of the basal-like subtype of invasive breast carcinoma” Clin Cancer Res. 10:5367-5374 (2004).
Paik, S., Shak, S., Tang, G., Kim, C., Baker, J., Cronin, M., Baehner, F. L., Walker, M. G., Watson, D., Park, T., et al. “A multigene assay to predict recurrence of tamoxifen-treated, node-negative breast cancer” N Engl J Med 351:2817-2826 (2004).
Panaro N J, Yuen P K, Sakazume T, Fortina P, Kricka L J, Wilding P. “Evaluation of DNA fragment sizing and quantification by the agilent 2100 bioanalyzer” Clin Chem 46:1851-1853 (2000).
Perou C M, Sorlie T, Eisen M B, van de Rijn M, Jeffrey S S, Rees C A, Pollack J R, Ross D T, Johnsen H, Akslen L A, Fluge O, Pergamenschikov A, Williams C, Zhu S X, Lonning P E, Borresen-Dale A L, Brown P O, Botstein D. “Molecular portraits of human breast tumours” Nature 406:747-752 (2000).
Perou C M, Brown P O, Botstein D. “Tumor classification using gene expression patterns from DNA microarrays” New Technologies for life sciences: A Trends Guide pp 67-76 (2000).
Perou, C. M., Jeffrey, S. S., van de Rijn, M., Rees, C. A., Eisen, M. B., Ross, D. T., Pergamenschikov, A., Williams, C. F., Zhu, S. X., Lee, J. C., et al. “Distinctive gene expression patterns in human mammary epithelial cells and breast cancers” Proc Natl Acad Sci USA 96:9212-9217 (1999).
Pinheiro J C BD. “Mixed-effects models in S and S-PLUS” New York, Springer (2000).
Pollack, J. R., Sorlie, T., Perou, C. M., Rees, C. A., Jeffrey, S. S., Lonning, P. E., Tibshirani, R., Botstein, D., Borresen-Dale, A. L., and Brown, P. O. “Microarray analysis reveals a major direct role of DNA copy number alteration in the transcriptional program of human breast tumors” Proc Natl Acad Sci USA 99:12963-12968 (2002).
Pollack, J. R., Perou, C. M., Alizadeh, A. A., Eisen, M. B., Pergamenschikov, A., Williams, C. F., Jeffrey, S. S., Botstein, D., and Brown, P. O. “Genome-wide analysis of DNA copy-number changes using cDNA microarrays” Nature Genetics 23:41-46 (1999).
Rasmussen R P. “Quantification on the LightCycler. In Rapid Cycle Real-Time PCR: Methods and Applications” Edited by Wittwer C T, Meuer, S., Nakagawara, K. Heidelberg, Springer Verlag, pp 21-34 (2001).
Robbins, P., Pinder, S., de Klerk, N., Dawkins, H., Harvey, J., Sterrett, G., Ellis, I., and Elston, C. “Histological grading of breast carcinomas: a study of interobserver agreement” Hum Pathol 26:873-879 (1995).
Ross, D. T., Scherf, U., Eisen, M. B., Perou, C. M., Rees, C., Spellman, P., Iyer, V., Jeffrey, S. S., Van de Rijn, M., Waltham, M., et al. “Systematic variation in gene expression patterns in human cancer cell lines [see comments]” Nat Genet 24:227-235 (2000).
Roux S, Pichaud F, Quinn J, Lalande A, Morieux C, Jullienne A, de Vernejoul M C. “Effects of prostaglandins on human hematopoietic osteoclast precursors” Endocrinology 138:1476-1482 (1997).
SantaLucia J. “A unified view of polymer, dumbbell, and oligonucleotide DNA nearest-neighbor thermodynamics” Proc Natl Acad Sci USA 95:1460-1465 (1998).
Schena M, Shalon D, Davis R W, Brown P O. “Quantitative monitoring of gene expression patterns with a complementary DNA microarray” Science 270:467-470 (1995).
Schwarz G. “Estimating the dimension of a model” The Annals of Statistics 6:461-464 (1978).
Singletary, S. E., Allred, C., Ashley, P., Bassett, L. W., Berry, D., Bland, K. I., Borgen, P. I., Clark, G. M., Edge, S. B., Hayes, D. F., et al. “Staging system for breast cancer” revisions for the 6th edition of the AJCC Cancer Staging Manual. Surg Clin North Am 83:803-819 (2003).
Sorlie, T., Tibshirani, R., Parker, J., Hastie, T., Marron, J. S., Nobel, A., Deng, S., Johnsen, H., Pesich, R., Geisler, S., et al. “Repeated observation of breast tumor subtypes in independent gene expression data sets” Proc Natl Acad Sci USA 100:8418-8423 (2003).
Sørlie, T., Perou, C. M., Tibshirani, R., Aas, T., Geisler, S., Johnsen, H., Hastie, T., Eisen, M. B., van de Rijn, M., Jeffrey, S. S., et al. “Gene expression patterns of breast carcinomas distinguish tumor subclasses with clinical implications” Proc Natl Acad Sci USA 98:10869-10874 (2001).
Sotiriou, C., Neo, S. Y., McShane, L. M., Korn, E. L., Long, P. M., Jazaeri, A., Martiat, P., Fox, S. B., Harris, A. L., and Liu, E. T. “Breast cancer classification and prognosis based on gene expression profiles from a population-based study” Proc Natl Acad Sci USA 100:10393-10398 (2003).
Spanakis E. “Problems related to the interpretation of autoradiographic data on gene expression using common constitutive transcripts as controls” Nucleic Acids Res 21:3809-3819 (1993).
Suzuki T, Higgins P J, Crawford D R. “Control selection for RNA quantitation” Biotechniques 29:332-337 (2000).
Szabo, A., Perou, C. M., Karaca, M., Perreard, L., Quackenbush, J. F., and Bernard, P. S. “Statistical modeling for selecting housekeeper genes” Genome Biol 5:R59 (2004).
Taylor-Papadimitriou, J., Stampfer, M., Bartek, J., Lewis, A., Boshell, M., Lane, E. B., and Leigh, I. M. “Keratin expression in human mammary epithelial cells cultured from normal and malignant tissue: relation to in vivo phenotypes and influence of medium” J Cell Sci 94:403-413 (1989).
Troyanskaya, O., Cantor, M., Sherlock, G., Brown, P., Hastie, T., Tibshirani, R., Botstein, D., and Altman, R. B. “Missing value estimation methods for DNA microarrays” Bioinformatics 17:520-525 (2001).
Tubbs R R, Pettay J D, Roche P C, Stoler M H, Jenkins R B, Grogan T M. “Discrepancies in clinical laboratory testing of eligibility for trastuzumab therapy: apparent immunohistochemical false-positives do not get the message” J Clin Oncol 19:2714-2721 (2001).
van de Vijver M J, He Y D, van't Veer L J, Dai H, Hart A A, Voskuil D W, Schreiber G J, Peterse J L, Roberts C, Marton M J, Parrish M, Atsma D, Witteveen A, Glas A, Delahaye L, van der Velde T, Bartelink H, Rodenhuis S, Rutgers E T, Friend S H, Bernards R. “A gene-expression signature as a predictor of survival in breast cancer” N Engl J Med 347:1999-2009 (2002).
van't Veer, L. J., Dai, H., van de Vijver, M. J., He, Y. D., Hart, A. A., Mao, M., Peterse, H. L., van der Kooy, K., Marton, M. J., Witteveen, A. T., et al. “Gene expression profiling predicts clinical outcome of breast cancer” Nature 415:530-536 (2002).
Vandesompele J, De Preter K, Pattyn F, Poppe B, Van Roy N, De Paepe A, Speleman F. “Accurate normalization of real-time quantitative RT-PCR data by geometric averaging of multiple internal control genes” Genome Biol 3:RESEARCH0034 (2002).
Welsh J B, Zarrinkar P P, Sapinoso L M, Kern S G, Behling C A, Monk B J, Lockhart D J, Burger R A, Hampton G M. “Analysis of gene expression profiles in normal and neoplastic ovarian tissue samples identifies candidate molecular markers of epithelial ovarian cancer” Proc Natl Acad Sci USA 98:1176-1181 (2001).
West, M., Blanchette, C., Dressman, H., Huang, E., Ishida, S., Spang, R., Zuzan, H., Olson, J. A., Jr., Marks, J. R., and Nevins, J. R. “Predicting the clinical status of human breast cancer by using gene expression profiles” Proc Natl Acad Sci USA 98:11462-11467 (2001).
Whitfield, M. L., Sherlock, G., Saldanha, A. J., Murray, J. I., Ball, C. A., Alexander, K. E., Matese, J. C., Perou, C. M., Hurt, M. M., Brown, P. O., et al. “Identification of genes periodically expressed in the human cell cycle and their expression in tumors” Mol Biol Cell 13:1977-2000 (2002).
Wittwer C T, a.K., N. “Real-time PCR. In Molecular Microbiology” T. Persing D H, F C, Versalovic, J, Tang, Y W, Unger, E R, Relman, D A, and White, T J, editor. Washington, D.C.: ASM Press (2004).
Yang, Y. H., Dudoit, S., Luu, P., Lin, D. M., Peng, V., Ngai, J., and Speed, T. P. “Normalization for cDNA microarray data: a robust composite method addressing single and multiple slide systematic variation” Nucleic Acids Res 30:e15 (2002).
Yu, K., Lee, C. H., Tan, P. H., and Tan, P. “Conservation of breast cancer molecular subtypes and transcriptional patterns of tumor progression across distinct ethnic populations” Clin Cancer Res 10:5508-5517 (2004).

What is claimed is:

1. A method of diagnosing cancer, the method comprising comparing expression levels of a nucleic acid comprising SEQ ID NO:1 to a test nucleic acid, wherein elevated expression of the test nucleic acid indicates a cancerous state.

2. A method of diagnosing cancer, the method comprising comparing expression levels of a nucleic acid comprising SEQ ID NO:1 and a nucleic acid comprising SEQ ID NO: 2 to a test nucleic acid, wherein elevated expression of the test nucleic acid indicates a cancerous state.

3. A method of quantitating level of expression of a test nucleic acid comprising: a) comparing gene expression levels of a nucleic acid comprising SEQ ID NO:1 to the test nucleic acid; and b) quantitating level of expression of the test nucleic acid.

4. A method of comparing expression levels of the same test nucleic acid expressed in multiple samples, comprising: a) co-amplifying a nucleic acid comprising SEQ ID NO:1 and the test nucleic acid; b) normalizing expression of the test nucleic acid amplified in each sample by i) comparing amplification of the nucleic acid comprising SEQ ID NO:1 across samples, and ii) applying normalization to the test nucleic acids; c) comparing expression levels of the test nucleic acids amplified across samples.

5. The method of claim 4, wherein the cancer is breast cancer.

6. The method of claim 4, wherein the cancer is colon cancer.

7. The method of claim 4, wherein the cancer is melanoma.

8. The method of claim 4, wherein said test nucleic acid is mRNA.

9. The method of claim 4, wherein the nucleic acid is amplified by PCR.

10. The method of claim 9, wherein the PCR is real time PCR.

11. A method of determining a total amount of mRNA in a sample comprising a) measuring expression level of a nucleic acid comprising SEQ ID NO:1; b) comparing the expression level of the nucleic acid comprising SEQ ID NO:1 to known values for percent of the nucleic acid comprising SEQ ID NO:1 of the total amount of mRNA; c) extrapolating the expression level of the nucleic acid comprising SEQ ID NO:1 to the total amount of mRNA; and d) determining the total amount of mRNA in the sample.

12. A method of normalizing the amount of mRNA amplified in multiple samples comprising a) comparing expression levels of a nucleic acid comprising SEQ ID NO:1 across multiple samples; b) deriving a value for normalizing expression of the nucleic acid comprising SEQ ID NO:1 across the multiple samples; and c) normalizing the expression of other nucleic acids amplified in the multiple samples based on the value obtained in step b).

13. A method of diagnosing cancer in a subject comprising: a) using a nucleic acid comprising SEQ ID NO:1 as a control; b) amplifying a sample comprising a nucleic acid indicative of cancer; c) determining if the control was amplified at an expected level, and if so, then d) determining if the nucleic acid indicative of cancer was also amplified, and if so then e) diagnosing cancer in the subject.

14. A method of classifying cancer in a subject, comprising: a) identifying intrinsic genes of the subject to be used to classify the cancer; b) obtaining a sample from the subject; c) amplifying and detecting levels of intrinsic genes in the subject; and d) classifying cancer based upon results of step c.

15. The method of claim 30, wherein qRT-PCR assay is used for step c.

16. The method of claim 30, wherein the cancer is breast cancer.

17. The method of claim 32, wherein the breast cancer is classified into luminal, normal-like, HER2+/ER−, and basal-like.

18. The method of claim 30, wherein the intrinsic gene set is identified using a microarray.

19. The method of claim 34, wherein the intrinisic gene set is modified from a microarray.

20. The method of claim 30, wherein the intrinisic gene set includes at least one housekeeper gene.

21. A method of prognosing outcome of a subject with cancer, comprising: a) amplifying and detecting prognostic genes; and b) prognosing the outcome based on expression levels of the gene within the subject.

22. The method of claim 21, wherein the prognostic genes are chosen from Table 10.

23. The method of claim 21, wherein the cancer is breast cancer.

24. A method of diagnosing cancer in a subject the method comprising: a) amplifying and detecting intrinsic genes; and b) diagnosing cancer based on expression levels of the gene within the subject.

25. The method of claim 24, wherein the intrinsic genes are chosen from Table 9.

26. The method of claim 24, wherein the cancer is breast cancer.

27. A kit comprising a nucleic acid, wherein the nucleic acid comprises SEQ ID NO: 1.

28. The kit of claim 21, wherein the kit also comprises a nucleic acid comprising SEQ ID NO: 2.

29. The kit of claim 21, wherein the kit also comprises a nucleic acid comprising SEQ ID NO: 3.

30. The kit of claim 22, wherein the kit also comprises a nucleic acid comprising SEQ ID NO: 3.

31. The kit of claim 21, wherein the kit also comprises a nucleic acid comprising SEQ ID NO: 4.

32. The kit of claim 22, wherein the kit also comprises a nucleic acid comprising SEQ ID NO: 4.

33. The kit of claim 23, wherein the kit also comprises a nucleic acid comprising SEQ ID NO: 4.

34. The kit of claim 24, wherein the kit also comprises a nucleic acid comprising SEQ ID NO: 4.

35. A kit comprising a nucleic acid, wherein the nucleic acid comprises SEQ ID NO: 2.

36. The kit of claim 29, wherein the kit also comprises a nucleic acid comprising SEQ ID NO: 3.

37. The kit of claim 29, wherein the kit also comprises a nucleic acid comprising SEQ ID NO: 4.

38. The kit of claim 30, wherein the kit also comprises a nucleic acid comprising SEQ ID NO: 4.

39. A kit comprising a nucleic acid, wherein the nucleic acid comprises SEQ ID NO: 3.

40. The kit of claim 33, wherein the kit also comprises a nucleic acid comprising SEQ ID NO: 4.

41. A kit comprising a nucleic acid, wherein the nucleic acid comprises SEQ ID NO: 4.

42. The kit of claim 21, wherein the kit also comprises instructions.

43. A method of diagnosing a disease in a subject, comprising: a) selecting one or more of housekeeper genes selected from Table 10, b) amplifying the housekeeper gene or genes from the subject using real-time qRT-PCR, c) amplifying classifier gene or genes from the subject used to classify the disease, d) normalizing gene expression of the classifier genes based on levels of the housekeeper genes; and e) diagnosing disease based on the normalized gene expression of the classifier genes.
